SEQUENCE LISTING (50 sequences)

SEQ ID NO: 1 - protein, 8 aa, synthetic construct
GFTFSSFG

SEQ ID NO: 2 - protein, 8 aa, synthetic construct
INSGSSTI

SEQ ID NO: 3 - protein, 9 aa, synthetic construct
TIYYYAMDY

SEQ ID NO: 4 - protein, 25 aa, synthetic construct
DVQLVESGGGLVQPGGSRKLSCAAS

SEQ ID NO: 5 - protein, 17 aa, synthetic construct
MHWVRQAPEKGLEWVAY

SEQ ID NO: 6 - protein, 38 aa, synthetic construct
NYADTVKGRFTISRDNPKNTLFLQMTSLRSEDTAMYYC

SEQ ID NO: 7 - protein, 11 aa, synthetic construct
WGQGTSVTVSS

SEQ ID NO: 8 - protein, 116 aa, synthetic construct
DVQLVESGGGLVQPGGSRKLSCAASGFTFSSFGMHWVRQAPEKGLEWVAYINSGSSTINYADTVKGRFTISRDNPKNTLFLQMTSLRSEDTAMYYCTIYYYAMDYWGQGTSVTVSS

SEQ ID NO: 9 - protein, 12 aa, synthetic construct
QTLLYSGNQKNY

SEQ ID NO: 10 - skipped

SEQ ID NO: 11 - protein, 10 aa, synthetic construct
QQYYIYPPWT

SEQ ID NO: 12 - protein, 26 aa, synthetic construct
DIVMSQSPSSLTVSVGEKVSMSCKSS

SEQ ID NO: 13 - protein, 17 aa, synthetic construct
LAWYQQKPGQSPKLLIY

SEQ ID NO: 14 - protein, 36 aa, synthetic construct
TRESGVPDRFAGSGSGTDFTLTISSVKAEDLAVYYC

SEQ ID NO: 15 - protein, 10 aa, synthetic construct
FGGGTKLEIK

SEQ ID NO: 16 - protein, 114 aa, synthetic construct
DIVMSQSPSSLTVSVGEKVSMSCKSSQTLLYSGNQKNYLAWYQQKPGQSPKLLIYWASTRESGVPDRFAGSGSGTDFTLTISSVKAEDLAVYYCQQYYIYPPWTFGGGTKLEIK

SEQ ID NO: 17 - protein, 255 aa, synthetic construct
DIVMSQSPSSLTVSVGEKVSMSCKSSQTLLYSGNQKNYLAWYQQKPGQSPKLLIYWASTRESGVPDRFAGSGSGTDFTLTISSVKAEDLAVYYCQQYYIYPPWTFGGGTKLEIKGGGGSGGGGSGGGGSGGGGSGGGGSDVQLVESGGGLVQPGGSRKLSCAASGFTFSSFGMHWVRQAPEKGLEWVAYINSGSSTINYADTVKGRFTISRDNPKNTLFLQMTSLRSEDTAMYYCTIYYYAMDYWGQGTSVTVSS

SEQ ID NO: 18 - DNA, 765 bp, synthetic construct
  source 1..765: mol_type = other DNA
gacattgtgatgtcacagtctccatcctccctaactgtgtcagttggagagaaggtttctatgagctgcaagtccagtcagacccttttatatagtggcaatcaaaagaactacttggcctggtaccagcagaaaccagggcagtctcctaaactgctgatttactgggcatccactagggaatctggggtccctgatcgcttcgcaggcagtggatctgggacagatttcactctcaccatcagcagtgtgaaggctgaagacctggcagtttattactgtcaacaatattatatctatcctccgtggacgttcggtggaggcaccaagctggaaatcaaaggagggggtggcagcggtggcgggggaagtggcggtggagggtcggggggaggcggttccgggggtggaggctcagatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattaatagtggcagtagtaccattaactatgcagacacagtgaagggccgattcaccatctccagagacaatcccaagaacaccctgttcctgcaaatgaccagtctaaggtctgaggacacggccatgtattactgtacaatctactactatgctatggactactggggtcaaggaacctcagtcaccgtctcctca

SEQ ID NO: 19 - protein, 245 aa, synthetic construct
DIVMSQSPSSLTVSVGEKVSMSCKSSQTLLYSGNQKNYLAWYQQKPGQSPKLLIYWASTRESGVPDRFAGSGSGTDFTLTISSVKAEDLAVYYCQQYYIYPPWTFGGGTKLEIKGGGGSGGGGSGGGGSDVQLVESGGGLVQPGGSRKLSCAASGFTFSSFGMHWVRQAPEKGLEWVAYINSGSSTINYADTVKGRFTISRDNPKNTLFLQMTSLRSEDTAMYYCTIYYYAMDYWGQGTSVTVSS

SEQ ID NO: 20 - DNA, 735 bp, synthetic construct
  source 1..735: mol_type = other DNA
gacattgtgatgtcacagtctccatcctccctaactgtgtcagttggagagaaggtttctatgagctgcaagtccagtcagacccttttatatagtggcaatcaaaagaactacttggcctggtaccagcagaaaccagggcagtctcctaaactgctgatttactgggcatccactagggaatctggggtccctgatcgcttcgcaggcagtggatctgggacagatttcactctcaccatcagcagtgtgaaggctgaagacctggcagtttattactgtcaacaatattatatctatcctccgtggacgttcggtggaggcaccaagctggaaatcaaaggagggggtggcagcggtggcgggggaagtggcggtggagggtcggatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattaatagtggcagtagtaccattaactatgcagacacagtgaagggccgattcaccatctccagagacaatcccaagaacaccctgttcctgcaaatgaccagtctaaggtctgaggacacggccatgtattactgtacaatctactactatgctatggactactggggtcaaggaacctcagtcaccgtctcctca

SEQ ID NO: 21 - protein, 255 aa, synthetic construct
DVQLVESGGGLVQPGGSRKLSCAASGFTFSSFGMHWVRQAPEKGLEWVAYINSGSSTINYADTVKGRFTISRDNPKNTLFLQMTSLRSEDTAMYYCTIYYYAMDYWGQGTSVTVSSGGGGSGGGGSGGGGSGGGGSGGGGSDIVMSQSPSSLTVSVGEKVSMSCKSSQTLLYSGNQKNYLAWYQQKPGQSPKLLIYWASTRESGVPDRFAGSGSGTDFTLTISSVKAEDLAVYYCQQYYIYPPWTFGGGTKLEIK

SEQ ID NO: 22 - DNA, 765 bp, synthetic construct
  source 1..765: mol_type = other DNA
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattaatagtggcagtagtaccattaactatgcagacacagtgaagggccgattcaccatctccagagacaatcccaagaacaccctgttcctgcaaatgaccagtctaaggtctgaggacacggccatgtattactgtacaatctactactatgctatggactactggggtcaaggaacctcagtcaccgtctcctcaggagggggtggcagcggtggcgggggaagtggcggtggagggtcggggggaggcggttccgggggtggaggctcagacattgtgatgtcacagtctccatcctccctaactgtgtcagttggagagaaggtttctatgagctgcaagtccagtcagacccttttatatagtggcaatcaaaagaactacttggcctggtaccagcagaaaccagggcagtctcctaaactgctgatttactgggcatccactagggaatctggggtccctgatcgcttcgcaggcagtggatctgggacagatttcactctcaccatcagcagtgtgaaggctgaagacctggcagtttattactgtcaacaatattatatctatcctccgtggacgttcggtggaggcaccaagctggaaatcaaa

SEQ ID NO: 23 - protein, 245 aa, synthetic construct
DVQLVESGGGLVQPGGSRKLSCAASGFTFSSFGMHWVRQAPEKGLEWVAYINSGSSTINYADTVKGRFTISRDNPKNTLFLQMTSLRSEDTAMYYCTIYYYAMDYWGQGTSVTVSSGGGGSGGGGSGGGGSDIVMSQSPSSLTVSVGEKVSMSCKSSQTLLYSGNQKNYLAWYQQKPGQSPKLLIYWASTRESGVPDRFAGSGSGTDFTLTISSVKAEDLAVYYCQQYYIYPPWTFGGGTKLEIK

SEQ ID NO: 24 - DNA, 735 bp, synthetic construct
  source 1..735: mol_type = other DNA
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattaatagtggcagtagtaccattaactatgcagacacagtgaagggccgattcaccatctccagagacaatcccaagaacaccctgttcctgcaaatgaccagtctaaggtctgaggacacggccatgtattactgtacaatctactactatgctatggactactggggtcaaggaacctcagtcaccgtctcctcaggagggggtggcagcggtggcgggggaagtggcggtggagggtcggacattgtgatgtcacagtctccatcctccctaactgtgtcagttggagagaaggtttctatgagctgcaagtccagtcagacccttttatatagtggcaatcaaaagaactacttggcctggtaccagcagaaaccagggcagtctcctaaactgctgatttactgggcatccactagggaatctggggtccctgatcgcttcgcaggcagtggatctgggacagatttcactctcaccatcagcagtgtgaaggctgaagacctggcagtttattactgtcaacaatattatatctatcctccgtggacgttcggtggaggcaccaagctggaaatcaaa

SEQ ID NO: 25 - protein, 15 aa, synthetic construct
GGGGSGGGGSGGGGS

SEQ ID NO: 26 - protein, 25 aa, synthetic construct
GGGGSGGGGSGGGGSGGGGSGGGGS

SEQ ID NO: 27 - DNA, 45 bp, synthetic construct
  source 1..45: mol_type = other DNA
ggtggaggcggttcaggtggcggcggttcgggcggtggcggctct

SEQ ID NO: 28 - protein, 22 aa, Homo sapiens
MLLLVTSLLLCELPHPAFLLIP

SEQ ID NO: 29 - DNA, 66 bp, Homo sapiens
  source 1..66: mol_type = other DNA
atgctgctgctcgtgacatctctgctgctgtgcgagctgccccaccccgcctttctgctgattcct

SEQ ID NO: 30 - protein, 229 aa, Homo sapiens
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 687 bp, Homo sapiens
  source 1..687: mol_type = other DNA
gagtctaagtacggccctccctgccccccctgcccagcccctgaatttgagggcggacccagcgtgttcctgttccccccaaagcccaaggacaccctgatgatcagccggacccccgaggtaacctgcgtggtggtggacgtgtcccaggaagatcccgaggtccagttcaattggtacgtggacggcgtggaagtgcacaatgccaagaccaagcccagagaggaacagttccaaagcacctaccgggtggtgtccgtgctgaccgtgctgcaccaggactggctgaacggcaaagagtacaagtgcaaggtgtccaacaagggcctgcccagcagcatcgagaaaaccatcagcaaggccaagggccagccccgcgagccccaggtgtacacactgccccccagccaggaagagatgaccaagaaccaggtgtccctgacctgcctggtcaagggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtctctgagcctgagcctgggcaag

SEQ ID NO: 32 - protein, 28 aa, Homo sapiens
MFWVLVVVGGVLACYSLLVTVAFIIFWV

SEQ ID NO: 33 - protein, 84 aa, Homo sapiens
ATGTTCTGGGTGCTGGTGGTGGTGGGCGGGGTGCTGGCCTGCTACAGCCTGCTGGTGACAGTGGCCTTCATCATCTTTTGGGTG

SEQ ID NO: 34 - protein, 41 aa, Homo sapiens
RSKRSRGGHSDYMNMTPRRPGPTRKHYQPYAPPRDFAAYRS

SEQ ID NO: 35 - DNA, 123 bp, Homo sapiens
  source 1..123: mol_type = other DNA
cggagcaagcggagcagaggcggccacagcgactacatgaacatgacccccagacggcctggccccacccggaagcactaccagccctacgccccacccagggactttgccgcctaccggtcc

SEQ ID NO: 36 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 336 bp, Homo sapiens
  source 1..336: mol_type = other DNA
agagtgaagttcagccggtccgccgacgcccctgcctaccagcagggccagaaccagctgtacaacgagctgaacctgggcaggcgggaggaatacgacgtgctggacaagcggagaggccgggaccctgagatgggcggcaagcccaggcggaagaaccctcaggaaggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgcccccaagg

SEQ ID NO: 38 - protein, 357 aa, synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 39 - DNA, 1071 bp, synthetic construct
  source 1..1071: mol_type = other DNA
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 40 - protein, 1058 aa, synthetic construct
MLLLVTSLLLCELPHPAFLLIPDVQLVESGGGLVQPGGSRKLSCAASGFTFSSFGMHWVRQAPEKGLEWVAYINSGSSTINYADTVKGRFTISRDNPKNTLFLQMTSLRSEDTAMYYCTIYYYAMDYWGQGTSVTVSSGGGGSGGGGSGGGGSDIVMSQSPSSLTVSVGEKVSMSCKSSQTLLYSGNQKNYLAWYQQKPGQSPKLLIYWASTRESGVPDRFAGSGSGTDFTLTISSVKAEDLAVYYCQQYYIYPPWTFGGGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 41 - DNA, 3177 bp, synthetic construct
  source 1..3177: mol_type = other DNA
atgctgctgctcgtgacatctctgctgctgtgcgagctgccccaccccgcctttctgctgattcctgatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattaatagtggcagtagtaccattaactatgcagacacagtgaagggccgattcaccatctccagagacaatcccaagaacaccctgttcctgcaaatgaccagtctaaggtctgaggacacggccatgtattactgtacaatctactactatgctatggactactggggtcaaggaacctcagtcaccgtctcctcaggtggaggcggttcaggtggcggcggttcgggcggtggcggctctgacattgtgatgtcacagtctccatcctccctaactgtgtcagttggagagaaggtttctatgagctgcaagtccagtcagacccttttatatagtggcaatcaaaagaactacttggcctggtaccagcagaaaccagggcagtctcctaaactgctgatttactgggcatccactagggaatctggggtccctgatcgcttcgcaggcagtggatctgggacagatttcactctcaccatcagcagtgtgaaggctgaagacctggcagtttattactgtcaacaatattatatctatcctccgtggacgttcggtggaggcaccaagctggaaatcaaagagtctaagtacggccctccctgccccccctgcccagcccctgaatttgagggcggacccagcgtgttcctgttccccccaaagcccaaggacaccctgatgatcagccggacccccgaggtaacctgcgtggtggtggacgtgtcccaggaagatcccgaggtccagttcaattggtacgtggacggcgtggaagtgcacaatgccaagaccaagcccagagaggaacagttccaaagcacctaccgggtggtgtccgtgctgaccgtgctgcaccaggactggctgaacggcaaagagtacaagtgcaaggtgtccaacaagggcctgcccagcagcatcgagaaaaccatcagcaaggccaagggccagccccgcgagccccaggtgtacacactgccccccagccaggaagagatgaccaagaaccaggtgtccctgacctgcctggtcaagggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtctctgagcctgagcctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctaccggtccagagtgaagttcagccggtccgccgacgcccctgcctaccagcagggccagaaccagctgtacaacgagctgaacctgggcaggcgggaggaatacgacgtgctggacaagcggagaggccgggaccctgagatgggcggcaagcccaggcggaagaaccctcaggaaggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 42 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 43 - DNA, 555 bp, Homo sapiens
  source 1..555: mol_type = other DNA
atgaggcgagggccccggagcctgcggggcagggacgcgccagcccccacgccctgcgtcccggccgagtgcttcgacctgctggtccgccactgcgtggcctgcgggctcctgcgcacgccgcggccgaaaccggccggggccagcagccctgcgcccaggacggcgctgcagccgcaggagtcggtgggcgcgggggccggcgaggcggcgctgcccctgcccgggctgctctttggcgcccccgcgctgctgggcctggcactggtcctggcgctggtcctggtgggtctggtgagctggaggcggcgacagcggcggcttcgcggcgcgtcctccgcagaggcccccgacggagacaaggacgccccagagcccctggacaaggtcatcattctgtctccgggaatctctgatgccacagctcctgcctggcctcctcctggggaagacccaggaaccaccccacctggccacagtgtccctgtgccagccacagagctgggctccactgaactggtgaccaccaagacggccggccctgagcaacaatag

SEQ ID NO: 44 - DNA, 348 bp, synthetic construct
  source 1..348: mol_type = other DNA
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattaatagtggcagtagtaccattaactatgcagacacagtgaagggccgattcaccatctccagagacaatcccaagaacaccctgttcctgcaaatgaccagtctaaggtctgaggacacggccatgtattactgtacaatctactactatgctatggactactggggtcaaggaacctcagtcaccgtctcctca

SEQ ID NO: 45 - DNA, 342 bp, synthetic construct
  source 1..342: mol_type = other DNA
gacattgtgatgtcacagtctccatcctccctaactgtgtcagttggagagaaggtttctatgagctgcaagtccagtcagacccttttatatagtggcaatcaaaagaactacttggcctggtaccagcagaaaccagggcagtctcctaaactgctgatttactgggcatccactagggaatctggggtccctgatcgcttcgcaggcagtggatctgggacagatttcactctcaccatcagcagtgtgaaggctgaagacctggcagtttattactgtcaacaatattatatctatcctccgtggacgttcggtggaggcaccaagctggaaatcaaa

SEQ ID NO: 46 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 47 - protein, 15 aa, synthetic construct
EPKSCDKTHTCPPCP

SEQ ID NO: 48 - DNA, 45 bp, synthetic construct
  source 1..45: mol_type = other DNA
gagcccaaatcttgtgacaaaactcacacatgcccaccgtgccca

SEQ ID NO: 49 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 651 bp, Homo sapiens
  source 1..651: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2026/0226185 A1
(43) Pub. Date: Aug. 6, 2026

(54) MOLECULES THAT BIND TO B-CELL ACTIVATING FACTOR RECEPTOR POLYPEPTIDES

(71) Applicant: Mayo Foundation for Medical Education and Research, Rochester, MN (US)

(72) Inventors: Hong Qin, Jacksonville, FL (US); Yan Luo, Ponte Vedra Beach, FL (US); Mohamed A. Kharfan Dabaja, Ponte Vedra Beach, FL (US)

(21) Appl. No.: 19/159,754

(22) PCT Filed: Feb. 28, 2024
(86) PCT No.: PCT/US2024/017732

§ 371 Date: Aug. 26, 2025

Related U.S. Application Data
(60) Provisional application No. 63/448,713, filed on Feb. 28, 2023.
(60) Provisional application No. 63/553,951, filed on Feb. 15, 2024.

(51) Int. Cl. C07K 16/28; A61K 40/33; A61K 40/41; A61P 37/06

(52) U.S. Cl. CPC C07K 16/2878; A61K 40/33; A61K 40/41; A61P 37/06; C07K 16/2818; C07K 2317/622

(57) ABSTRACT

This document provides methods and materials involved in binding a binder (e.g., an antibody, antigen binding fragment, antibody domain, CAR, cell engager, and/or ADC) to a BAFF-R polypeptide. For example, binders (e.g., antibodies, antigen binding fragments, antibody domains, CARs, cell engagers, and/or ADCs) that bind to a BAFF-R polypeptide and methods and materials for using one or more such binding molecules to treat a mammal (e.g., a human) having cancer (e.g., one or more B-cell cancers) are provided.

### CROSS-REFERENCE TO RELATED APPLICATIONS

This application claims the benefit of U.S. Patent Application Ser. No. 63/448,713, filed on Feb. 28, 2023, and U.S. Patent Application Ser. No. 63/553,951, filed on Feb. 15, 2024. The disclosure of the prior application is considered part of, and is incorporated by reference in, the disclosure of this application.

### SEQUENCE LISTING

This application contains a Sequence Listing that has been submitted electronically as an XML file named “07039-2188WO1_SL.xml.” The XML file, created on Feb. 27, 2024, is 57000 bytes in size. The material in the XML file is hereby incorporated by reference in its entirety.

### TECHNICAL FIELD

This document relates to methods and materials involved in binding a molecule (e.g., an antibody, a fragment of an antibody, an antibody domain, a chimeric antigen receptor (CAR), a cell engager, or an antibody-drug conjugate (ADC)) to a B-cell activating factor receptor (BAFF-R) polypeptide. For example, this document provides binders (e.g., antibodies, antigen binding fragments, antibody domains, CARs, cell engagers, or ADCs) that bind to a BAFF-R polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a BAFF-R polypeptide and methods and materials for using such cells to treat cancer.

### BACKGROUND INFORMATION

Immunotherapies can be non-specific (e.g., interleukins and cytokines) or specific (e.g., monoclonal antibodies and T cells expressing CARs (CAR-T cells)). Immunotherapies have current applications in treating cancers; however, refractory and relapse disease, as high as 50% for some malignancies, have been observed following CAR-T cell therapies. Both antigen-positive relapse and antigen-negative relapse (e.g., loss or reduction of an antigen expressed on the malignant cells) cancers have been observed.

### SUMMARY

This document provides methods and materials involved in binding a molecule (e.g., an antibody, an antigen binding fragment, an antibody domain, a CAR, a cell engager, or an ADC) to a BAFF-R polypeptide. For example, this document provides binders (e.g., antibodies, antigen binding fragments, antibody domains, CARs, cell engagers, or ADCs) that bind to a BAFF-R polypeptide and methods and materials for using one or more such binders to treat a mammal (e.g., a human) having cancer.

This document also provides cells (e.g., host cells) designed to express one or more binders (e.g., antibodies, antigen binding fragments, antibody domains, CARs, or cell engagers) having the ability to bind to a BAFF-R polypeptide and methods and materials for using such cells to treat cancer.

As described herein, binders (e.g., one or more antibodies, one or more antigen binding fragments, one or more antibody domains, one or more CARs, one or more cell engagers, and/or one or more ADCs) can be designed to have the ability to bind to a BAFF-R polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human BAFF-R polypeptide as set forth in SEQ ID NO:42 (see, e.g., Example 2).

In some cases, two sets of three complementarity-determining regions (CDRs) of an antigen binding fragment provided herein (e.g., SEQ ID NOs:1-3 and 9-11) can be engineered into a CAR and used to create CAR⁺ cells (e.g., CAR⁺ T cells, CAR⁺ stem cells such as CAR⁺ induced pluripotent stem cells, or CAR⁺ natural killer (NK) cells) having the ability to target BAFF-R⁺ cells (e.g., BAFF-R⁺ tumor cells), can be engineered into an antibody structure that includes an Fc region to create antibodies having the ability to target BAFF-R⁺ cells (e.g., BAFF-R⁺ tumor cells) and induce antibody-dependent cell-mediated cytotoxicity (ADCC) against the target BAFF-R⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target BAFF-R⁺ cells (e.g., BAFF-R⁺ tumor) and induce one or more immune responses (e.g., T cell immune responses and/or ADCC using a cell engager in the presence or absence of an Fc-containing antibody) against the target BAFF-R⁺ cells. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as cancer cells (e.g., BAFF-R⁺ cancer cells).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer (e.g., one or more B-cell cancers). For example, a mammal (e.g., a human) having cancer (e.g., a BAFF-R⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

As also described herein, cells (e.g., host cells) can be designed to express one or more binders (e.g., antibodies, antigen binding fragments, antibody domains, CARs, or cell engagers) having the ability to bind to a BAFF-R polypeptide. For example, cells such as T cells (e.g., CTLs), stem cells (e.g., induced pluripotent stem cells), or NK cells can be engineered to express one or more CARs having the ability to bind to a BAFF-R. Such cells (e.g., BAFF-R-specific CAR⁺ T cells or NK cells) can be used to treat cancer (e.g., one or more B-cell cancers).

In general, one aspect of this document features antibodies including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO:11 (or SEQ ID NO:11 with one, two, or three amino acid additions, deletions, or substitutions). The antibody can have the ability to bind to SEQ ID NO:42. The heavy chain variable domain or region can include an amino acid sequence having at least 85 percent identity to the amino acid sequence set forth in SEQ ID NO:8. The light chain variable domain or region can include an amino acid sequence having at least 85 percent identity to the amino acid sequence set forth in SEQ ID NO: 16. The antibody can be a monoclonal antibody. The antibody can be an scFv antibody.

In another aspect, this document features antigen binding fragments including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO:11 (or SEQ ID NO:11 with one, two, or three amino acid additions, deletions, or substitutions). The antigen binding fragment can have the ability to bind to SEQ ID NO:42. The heavy chain variable domain or region can include an amino acid sequence having at least 85 percent identity to the amino acid sequence set forth in SEQ ID NO:8. The light chain variable domain or region can include an amino acid sequence having at least 85 percent identity to the amino acid sequence set forth in SEQ ID NO: 16. The antigen binding fragment can be monoclonal. The antigen binding fragment can be a Fab.

In another aspect, this document features chimeric antigen receptors including an antigen binding domain, a hinge, a transmembrane domain, and one or more signaling domains, where the antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The antigen binding domain can include a scFv having the ability to bind to a BAFF-R polypeptide. The hinge can include an amino acid sequence set forth in SEQ ID NO:30. The transmembrane domain can include an amino acid sequence set forth in SEQ ID NO:32. The chimeric antigen receptor comprises one or more signaling domains set forth in any one of SEQ ID NOs:34 and 36.

In another aspect, this document features cells including a chimeric antigen receptor including an antigen binding domain, a hinge, a transmembrane domain, and one or more signaling domains, where the antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The cell can be a T cell, a stem cell, or an NK cell.

In another aspect, this document features cell engagers including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The first antigen binding domain can include a scFv having the ability to bind to a BAFF-R polypeptide. The first antigen binding domain can be an IgG having the ability to bind to a BAFF-R polypeptide. The linker comprises a linker set forth in any one of SEQ ID NOs:25, 26, and 30. The second antigen binding domain can bind to a polypeptide expressed on the surface of T cells (e.g., a CD3 polypeptide). The second antigen binding domain can bind to a polypeptide expressed on the surface of NK cells (e.g., a CD16a, NKG2A, NKG2D, NKp30, NKp44, NKp46, or CRTAM polypeptide). The cell engager can include a third antigen binding domain. The third antigen binding domain can bind to a polypeptide expressed on the surface of NK cells (e.g., a CD16a, NKG2A, NKG2D, NKp30, NKp44, NKp46, or CRTAM polypeptide).

In another aspect, this document features nucleic acids including a nucleic acid sequence encoding at least part of an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The nucleic acid sequence can encode a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions). The nucleic acid sequence can encode a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The nucleic acid can be a viral vector. The nucleic acid can be a phagemid.

In another aspect, this document features nucleic acids including a nucleic acid sequence encoding a chimeric antigen receptor including an antigen binding domain, a hinge, a transmembrane domain, and one or more signaling domains, where the antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions) or a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO:11 (or SEQ ID NO:11 with one, two, or three amino acid additions, deletions, or substitutions). The nucleic acid can be a viral vector. The nucleic acid can be a phagemid.

In another aspect, this document features host cells including a nucleic acid sequence encoding a chimeric antigen receptor including an antigen binding domain, a hinge, a transmembrane domain, and one or more signaling domains, where the antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO:11 (or SEQ ID NO:11 with one, two, or three amino acid additions, deletions, or substitutions) or a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions).

In another aspect, this document features host cells that can express a chimeric antigen receptor of including an antigen binding domain, a hinge, a transmembrane domain, and one or more signaling domains, where the antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO: 11 (or SEQ ID NO:11 with one, two, or three amino acid additions, deletions, or substitutions) or a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO: 11 (or SEQ ID NO:11 with one, two, or three amino acid additions, deletions, or substitutions). The host cell can be a T cell, stem cell, or NK cell.

In another aspect, this document features ADCs including an antigen binding domain covalently linked to a drug, where the antigen binding domain comprises an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO: 11 (or SEQ ID NO:11 with one, two, or three amino acid additions, deletions, or substitutions). The antigen binding domain can include a scFv having the ability to bind to a BAFF-R polypeptide. The antigen binding domain can be an IgG having the ability to bind to a BAFF-R polypeptide. The drug can be selected from the group consisting of auristatins, mertansine, or pyrrolobenzodiazepine (PBD) dimers.

In another aspect, this document features compositions including an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The composition can include the antibody. The composition can include the antigen binding fragment. The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features compositions including a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features compositions including cells expressing chimeric antigen receptors featured herein. The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features compositions including ADCs featured herein. The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features methods of treating a mammal having cancer. The methods can include, or consist essentially of, administering, to a mammal having cancer, a composition including an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO:11 (or SEQ ID NO:11 with one, two, or three amino acid additions, deletions, or substitutions) or a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO:11 (or SEQ ID NO:11 with one, two, or three amino acid additions, deletions, or substitutions). The mammal can be a human. The cancer can be a BAFF-R⁺ cancer. The BAFF-R⁺ cancer can be a chronic lymphocytic leukemia (CLL), an acute lymphoblastic leukemia (ALL), a hairy cell leukemia, a follicular lymphoma, a non-Hodgkin's lymphoma (NHL), a Hodgkin's lymphoma, a multiple myeloma, a Waldenstrom's macroglobulinemia, a diffuse large B-cell lymphoma (DLBCL), or an intravascular large B-cell lymphoma. The number of cancer cells within the mammal can be reduced following the administering step.

In another aspect, this document features methods for treating a mammal having cancer. The methods can include, or consist essentially of, (a) administering, to a mammal having cancer, a composition including an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO:11 (or SEQ ID NO:11 with one, two, or three amino acid additions, deletions, or substitutions) or a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO:11 (or SEQ ID NO:11 with one, two, or three amino acid additions, deletions, or substitutions), and (b) administering, to the mammal, a composition comprising a checkpoint inhibitor. The mammal can be a human. The cancer can be a BAFF-R⁺ cancer. The BAFF-R⁺ cancer can be a CLL, an ALL, a hairy cell leukemia, a follicular lymphoma, a NHL, a Hodgkin's lymphoma, a multiple myeloma, a Waldenström's macroglobulinemia, a DLBCL, or an intravascular large B-cell lymphoma. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab. The number of cancer cells within the mammal can be reduced following the administering steps (a) and (b).

In another aspect, this document features methods for binding a binding molecule to a BAFF-R polypeptide. The methods can include, or consist essentially of, contacting a BAFF-R polypeptide with an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The contacting can be performed in vitro. The contacting can be performed in vivo. The contacting can be performed within a mammal by administering the antibody or the antigen binding fragment to the mammal. The mammal can be a human.

In another aspect, this document features methods for binding a binding molecule to a BAFF-R polypeptide. The methods can include, or consist essentially of, contacting a BAFF-R polypeptide with a chimeric antigen receptor including an antigen binding domain, a hinge, a transmembrane domain, and one or more signaling domains, where the antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions), a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO:11 (or SEQ ID NO:11 with one, two, or three amino acid additions, deletions, or substitutions), or an ADC including an antigen binding domain covalently linked to a drug, where the antigen binding domain comprises an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO:10 with one, two, or three amino acid additions, deletions, or substitutions), and SEQ ID NO:11 (or SEQ ID NO:11 with one, two, or three amino acid additions, deletions, or substitutions). The contacting can be performed in vitro. The contacting can be performed in vivo. The contacting can be performed within a mammal by administering the chimeric antigen receptor, the cell engager, or the ADC to the mammal. The mammal can be a human.

In another aspect, this document features methods of treating a mammal having a transplanted organ. The methods can include, or consist essentially of, administering, to a mammal having a transplanted organ, a composition including an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions) or a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions). The mammal can be a human. The transplanted organ can be a kidney, a lung, a liver, a heart, a pancreas, or bone marrow.

In another aspect, this document features methods of delaying or preventing organ transplant rejection in a mammal having or preparing to have an organ transplant. The methods can include, or consist essentially of, administering, to a mammal having or preparing to have an organ transplant, a composition including an antibody or an antigen binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions) or a cell engager including a first antigen binding domain, a linker, and a second antigen binding domain, where the first antigen binding domain comprises an antibody or an antigen-binding fragment including: a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 11 with one, two, or three amino acid additions, deletions, or substitutions), thereby delaying or preventing organ transplant rejection in the mammal. The mammal can be a human. The organ transplant can be a kidney transplant, a lung transplant, a liver transplant, a heart transplant, a pancreas transplant, or a bone marrow transplant.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E show the generation of an anti-BAFF-R antibody. FIGS. 1A-1B) BAFF-R-pCDH lentivirus infected NIH/3T3 or 293FT cells were selected with puromycin, followed by cell sorting. The expression of BAFF-R in BAFF-R-NIH/3T3 single-cell clone (FIG. 1A) or BAFF-R-293FT cell clone (FIG. 1B) was confirmed by flow cytometry. FIG. 1C) Crude hybridoma supernatants were screened for antigen specific binding affinity to BAFF-R-positive and BAFF-R-negative 293FT with serial diluted concentration to generate this representative data. FIG. 1D) Antigen specific binding for crude C21 supernatant. FIG. 1E) Antigen specific binding affinity of purified mAb C21 to BAFF-R-positive and BAFF-R-negative 293FT at a concentration of 0.002 mg mAb/10⁶ cells. The data are representative of three independent experiments.

FIGS. 2A-2E show a MC10029 CAR construct and initial characterization of MC10029 CAR-T cell production. FIG. 2A) Schematic diagram showing the order of functional elements within the CAR design. The construct included the scFv of our new BAFF-R antibody with the following elements in tandem: promoter (EF1P), signal peptide (SP), BAFF-R scFv, hinge, CD28 transmembrane (TM) domain, CD28 costimulatory domain (CD28QQ), CD3ζ, T2A (ribosomal skipping site that arrests translation), and truncated epidermal growth factor receptor (tEGFR or EGFR). (FIG. 2B) Fold expansion of CAR-T cells was calculated from day 1 to day 14 by measuring the number of viable CAR-T cells by trypan blue exclusion using Bio-Rad Cell counter. Non-transduced T cells from the same donor were used as controls. FIGS. 2C-2D) MC10029 CAR-T cells were stained with antibodies to measure surface expression of CD3 for potency (FIG. 2C) or EGFR (FIG. 2D) to characterize the CAR-T cells with the data shown in these representative flow cytometry dot plots. FIG. 2E) Quality control assays were performed on three production batches of MC10029 CAR-T cells that were used in various in vitro assays.

FIGS. 3A-3B show the characterization of antigen expression on malignant B-cell lines used for MC10029 CAR-T cell characterization. Three cell lines that were used to evaluate antigen specific cytotoxicity of MC10029 CAR-T cells were tested for BAFF-R surface expression. (FIG. 3A) Using anti-BAFF-R-AF647 antibody, flow cytometry histograms show BAFF-R expression in Nalm-6, Z-138, or MEC-1. (FIG. 3B) These cell lines were also evaluated for CD19 surface expression, and a CD19 knock-out of Nalm-6 (CD19 KO Nalm-6) was generated and evaluated for antigen surface expression. Flow cytometry histograms show BAFF-R or CD19 expression in wild type Nalm-6, Z-138, or MEC-1 cell lines, as well as the CD19 knock out (KO) Nalm-6 cell line. Cells were stained by BAFF-R-AF647 or CD19 allophycocyanin (APC).

FIGS. 4A-4D show antigen-specific cytotoxicity of MC10029 CAR-T cells targeting acute lymphocytic leukemia (ALL) cell lines. FIGS. 4A and 4B) Flow cytometry contour plots of CAR-T cell functional potency as measured by the surface expression of CD107a in a degranulation assay. MC10029 CAR-T cells were incubated with wild type Nalm-6 cells (Nalm-6 WT) or Nalm-6 BAFF-R knock out (CD19 KO BAFF-R) cells at an E:T ratio of 2:1. Analysis was gated on CD4⁺ BAFF-R CAR-T cells (FIG. 4A) or gated on CD8⁺ BAFF-R CAR-T cells (FIG. 4B). Gating on epidermal growth factor receptor (EGFR) was used as a surrogate for BAFF-R. Non-transduced (non-CAR) T cells from the same donor were used as negative controls. FIG. 1C) Direct target cell cytolysis measured by incubating green fluorescent protein (GFP) expressing Nalm-6 cells with CAR-T cells. MC10029 CAR-T cells were co-incubated with either Nalm-6 WT or Nalm-6 BAFF-R KO cells at an E:T ratio of 20:1 for 24 hours. This was followed by flow cytometry to quantify the GFP expression target cells. The results shown are representative from three independent experiments. FIG. 4D) Granzyme B enzyme-linked immunosorbent assay (ELISA) further confirmed the antigen specific potency of MC10029 CAR-T cells targeting Nalm-6 cells. MC10029 CAR-T cells or non-transduced T cells generated from the same donor were co-incubated with either Nalm-6 WT or Nalm-6-BAFF-R KO cells at an E:T ratio of 4:1. After 72 hours, the supernatants were collected, and granzyme B was measured by ELISA. Graphed data are means of quadruplicate sampling. The results shown are representative of three independent experiments.

FIGS. 5A-5B show that MC10029 CAR-T cells elicit in vivo antitumor effects on Nalm-6 ALL tumors. FIG. 5A) The therapeutic efficacy of BAFF-R CAR-T cells was tested in a NOD scid gamma (NSG) mouse model in which the mice were challenged with luciferase labeled-human Nalm-6 tumor cells (Nalm-6-Luc, 0.25×10⁶ cells). Bioluminescent imaging catalogues the changes of luciferase-expressing Nalm-6 ALL tumor cells. Six days after tumor cell injection, tumor-bearing mice were randomized into three groups (N=5 per group) to receive a single intravenous (IV) infusion of either vehicle (PBS), non CAR-T cells (10×10⁶ cells), or BAFF-R CAR-T cells (2×10⁶ cells) that were generated from the same donor. Although imaged weekly, we share representative images along the time course to highlight changes due to treatment. These data are the representative of two independent experiments using different donor T cells. FIG. 5B) A Kaplan-Meier plot shows overall survival versus days after tumor challenge, and log rank analysis identified statistical differences between the treatment groups.

FIGS. 6A-6D show the characterization of in vitro and in vivo cytotoxicity of MC10029 CAR-T cells targeting a CD19 deficient Nalm-6 cell line. FIG. 6A) Flow cytometry contour plots that show the functional potency of CAR-T cells against target cells by the surface expression CD107a in a degranulation assay. Non-transduced T cells, MC10029 CAR-T cells, or CD19 CAR-T cells were generated from the same donor and incubated with CD19 KO-Nalm-6 cells at an E:T ratio of 2:1 to identify CD19 antigen-specific cytotoxicity. Analysis was gated on CD8+ CAR-T cell populations. FIG. 6B) Granzyme B ELISA shows the functional potency of MC10029 CAR-T cells targeting CD19 KO Nalm-6 cells. Non-transduced T cells, MC10029 CAR-T cells, or CD19 CAR-T cells were co-incubated with either WT or Nalm-6-CD19 KO cells at an E:T ratio of 4:1 for 72 hours when the supernatants were harvested for subsequent ELISA Graphed data are means of quadruplicate sampling. The data are representative of three independent experiments. FIG. 6C) Bioluminescent imaging catalogues that show the changes of luciferase-expressing CD19 deficient-Nalm-6 model (CD19 KO-Nalm6-Luc, 0.25×10⁶ cells) that were injected into NSG mice followed by treatment with one of four treatments. Six days after tumor cell injection, tumor-bearing mice were randomized into four groups (N=5 per group). On day seven, mice received single infusion treatment with either PBS (vehicle), non-transduced T cell from same donor (10×10⁶ cells), MC10029 CAR-T cells (2×10⁶ cells), or CD19 CAR-T cells (2×10⁶ cells). Tumor burden was quantified by bioluminescence intensity. The results shown are the representative of two independent experiments using different donor T cells. FIG. 6D) A Kaplan-Meier plot shows overall survival versus days after tumor challenge, and log rank analysis identified statistical differences between the treatment groups.

FIGS. 7A-7D show the characterization of in vitro and in vivo cytotoxicity of MC10029 CAR-T cells targeting Z138, a lymphoma cell line. FIG. 7A) Flow cytometry contour plots that show the functional potency of CAR-T cells against target cells by the surface expression of CD107a in a degranulation assay. Non-transduced T cells and MC10029 CAR-T cells were generated from the same donor and incubated with Z138 cells at an E:T ratio of 2:1 to characterize antigen-specific cytotoxicity. Analysis was gated on CD8⁺ CAR-T cell populations. FIG. 7B) Granzyme B ELISA that shows the functional potency of MC10029 CAR-T cells targeting Z138 cells. Non-transduced T cells or MC10029 CAR-T cells were co-incubated Z138 cells at an E:T ratio of 4:1 for 72 hours, when the supernatants were harvested for subsequent ELISA. Graphed data are means of quadruplicate sampling. The data are representative of three independent experiments. FIG. 7C) Bioluminescent imaging catalogues that show the changes of luciferase-expressing Z138 model (Z138-Luc, 0.5×10⁶ cells) that were injected into NSG mice that were subsequently treated with MC10029 CAR-T cells. Six days after tumor cell injection, tumor-bearing mice were randomized into three groups (N=5 per group). On day seven, mice received single infusion treatment with either PBS (vehicle), Non CAR-T cells (non-transduced T cell) from same donor (10×10⁶ cells), or MC10029 CAR-T cells (2×10⁶ cells). Tumor burden was quantified by bioluminescence intensity. The results shown are the representative of two independent experiments using different donor T cells. FIG. 7D) A Kaplan-Meier plot that shows an overall survival versus days after Z138 tumor challenge, and log rank analysis identified statistical differences between the treatment groups.

FIGS. 8A-8D show the characterization of in vitro and in vivo cytotoxicity of MC10029 CAR-T cells targeting MEC-1, a chronic lymphocytic leukemia (CLL) cell line. FIG. 8A) Flow cytometry contour plots that show the functional potency of CAR-T cells against target cells by the surface expression CD107a in a degranulation assay. Non-transduced T cells and MC10029 CAR-T cells were generated from the same donor and incubated with MEC-1 cells at an E:T ratio of 2:1 to characterize antigen-specific cytotoxicity. Analysis was gated on CD8⁺ CAR-T cell populations. FIG. 8B) Granzyme B ELISA assay that shows functional potency of MC10029 CAR-T cells targeting MEC-1. Non-transduced T cells or MC10029 CAR-T cells were co-incubated MEC-1 cells at an E:T ratio of 4:1 for 72 hours, when the supernatants were harvested for subsequent ELISA. Graphed data are means of quadruplicate sampling. The data are representative of three independent experiments. FIG. 8C) Bioluminescent imaging catalogues that show the changes of luciferase-expressing MEC-1 model (MEC-1-Luc, 1.0×10⁶ cells) that were injected into NSG mice followed by treatment with one of three treatments. Six days after tumor cell injection, tumor-bearing mice were randomized into three groups (N=5 per group). On day seven, mice received single infusion treatment with either PBS (vehicle), non-transduced T cell from same donor (10×10⁶ cells), or MC10029 CAR-T cells (2×10⁶ cells). Tumor burden was quantified by bioluminescence intensity. The results shown are the representative of two independent experiments using different donor T cells. FIG. 8D) A Kaplan-Meier plot that shows overall survival versus days after MEC-1 tumor challenge, and log rank analysis identified statistical differences between the treatment groups.

FIGS. 9A-9C show that MC10029 CAR-T cells elicited in vitro cytotoxicity targeting primary CLL tumors. FIG. 9A) A flow cytometry assay that shows the cell surface expression of BAFF-R peripheral blood mononuclear cells (PBMCs) that were collected from six subjects with CLL. Fluorescence minus one (FMO) staining was a setting for the gating strategy. FIG. 9B) The enriched primary B-cell tumor samples from six subjects were incubated with MC10029 CAR-T cells from two different healthy donors at an E:T ratio of 2:1. Using a CD107a degranulation assay, the cytotoxic activity of MC10029 CAR-T cells was visualized using flow cytometry. Analysis was gated on CD8⁺ CAR-T cell populations. Non-transduced T cells from the same donor were used as a negative control. FIG. 9C) The release of granzyme B confirmed the functional potency of MC10029 CAR-T cells targeting primary CLL tumor cells. MC10029 CAR-T cells or non-transduced T cells were co-incubated with enriched primary tumor cells isolated from subjects with CLL (E:T ratio of 4:1) for 72 hours. Granzyme B was measured from the harvested supernatants. Graphed data are means of quadruplicate sampling.

FIG. 10. Controls were performed to ensure the quality of both the B cells that were used and the CAR-T cells that were generated. To confirm that MC10029 CAR-T cell activity was targeting primary CLL tumor cells, the PBMCs were enriched for tumor cells, CLL B-cells . . . Using flow cytometry, the population of CD3 positive cells from six subjects with CLL were characterized in the original PBMC samples (FIG. 10, top panels) and after B-cells enrichment (FIG. 10, bottom panels) to confirm endogenous T cell removal. Cells were stained by CD3-BV605.

FIGS. 11A-11C show the characterization of MC10029 CAR-T cells used to target primary CLL tumor cells and that elicited the responses shown in FIG. 9B-9C. MC10029 CAR-T cells were generated from two healthy donors. The two production batches of MC10029 CAR-T cells were characterized using standard QC assays. The identity (CD3 positive cells) and the potency (quantity of EGFR) of MC10029 CAR-T cell from donor A (FIG. 11A) were nearly identical as those from donor B (FIG. 11B). Although fold expansion (final total number of MC10029 CAR-T cells) differed (FIG. 11C) between the two batches, the fold expansion of both allowed for comparison of the two batches of MC10029 CAR-T cells in the in vitro assays.

FIGS. 12A-12B show quality control assay results of three engineering productions of clinical-grade MC10029 CAR-T cells. FIG. 12A) Quality control product release criteria for batches of CAR-T cells. Three batches of MC10029 CAR-T cells and their corresponding non CAR-T cells as controls were evaluated for cell quality with fold expansion (>25) and viability (>70%); and CAR-T cell specific characterization with identity (>80%) and potency (>10%). To show that the lentiviral vector remains non-infectious and safe from adventitious viral agents, the lentiviral copy number (<5 copies/per cell of WPRE) was determined by real-time qPCR method and vesicular stomatitis virus G glycoprotein (VSVG) was assayed using a qPCR assay (<5 copies/50 ng DNA). FIG. 12B) Flow cytometry contour plots of CAR-T cell functional potency as measured by a CD107a degranulation assay. MC10029 CAR-T cells (characterized in FIG. 12A) from three different healthy donors were incubated with Nalm-6 WT or Nalm-6 BAFF-R KO at an E:T ratio of 2:1. Analysis was gated on CD8⁺ T cell populations. Non-transduced T cells from the same donor were used as negative controls.

FIG. 13 is a schematic of an exemplary vector that can encode a C21-BAFF-R 10029 CAR.

FIG. 14 is a schematic of an exemplary BiTE designed using CDR1, CDR2, and CDR3 of a heavy chain provided herein and CDR1, CDR2, and CDR3 of a light chain provided herein in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a linker set forth in in Example 10 such as a (G₄S)₃ (SEQ ID NO:25) linker).

FIGS. 15A-15C show that subject-derived MC10029 CAR-T cells elicited ex vivo cytotoxicity against autologous CLL tumors. FIG. 15A) The growth curves of MC10029 CAR-T cells and their corresponding Non-CAR-T cells, both derived from T cells isolated from the peripheral blood of three CLL subjects, were plotted side by side to compare their growth patterns. FIG. 15B) The subject-derived MC10029 CAR-T cells were assessed for their identity (>80% CD3 positive cells) and potency (>10% EGFR positive cells). FIG. 15C) The cytotoxicity of MC10029 CAR-T cells derived from the three subjects with CLL was evaluated against autologous tumor cells using a CD107a degranulation assay. MEC-1 cells were employed as a positive control, while the corresponding Non-CART cells served as a negative control. The analysis of cytotoxicity was gated on the CD8+ CAR-T cell populations.

FIGS. 16A-16B show characterization of three qualification productions of clinical-grade MC10029 CAR-T cells. FIG. 16A) Product release criteria for CAR-T cells. Three batches of MC10029 CAR-T cells were evaluated for cell quality with fold expansion (>25) and viability (>70%, as determined by Trypan Blue staining) as well as CAR-T cell specific characterization with identity (>80%, as determined by flow cytometry for CD3 positive cells) and potency (>10%, as determined by flow cytometry for EGFR (a transgene) positive T cells). The corresponding non-CAR-T cells were used as controls. To show that the lentiviral vector remains non-infectious and safe from adventitious viral agents, the lentiviral copy number (<5 copies/per cell of WPRE) and VSVG (<5 copies/50 ng DNA) were determined using a qPCR assay. FIG. 16B) Flow cytometry contour plots of CAR-T cell functional potency as measured by a CD107a degranulation assay. MC10029 CAR-T cells (characterized in panel A) from three different healthy donors were incubated with WT Nalm-6 or BAFF-R KO Nalm-6 at an E:T ratio of 2:1. Analysis was gated on CD8+ CAR-T cell populations and showed no difference between the batches (FIG. 20F). Non-transduced T cells (Non-CAR-T) from the same donor were used as negative controls.

FIGS. 17A-17C show a comparison of BAFF-R CAR-T cell constructs with differing costimulatory domains. With the option of complementing CD3ζ (with either the CD28 costimulatory domain (MC10029) or the 4-1 BB costimulatory domain (MC10023), both CARs were generated and transduced into T cells to identify which BAFF-R CAR-T cells provided superior antigen specific cytotoxicity. FIG. 17A) Expansion of MC10023 and MC10029 CAR-T cells showed comparable fold increases by day 14. FIG. 17B) The T cell content (CD3) and the proportion of CAR-T cells (using EGFR as a proxy) within the final products of both MC10029 and MC10023 CAR-T cells are also found to be comparable. FIG. 17C) Antigen specific cytotoxicity was assayed by testing MC10023 CAR-T cells or MC10029 CAR-T cells against Nalm-6 and Nalm-6 BAFF-R KO cell lines; this degranulation assay shows the higher activity of MC10029 CAR-T cells compared to MC10023 CAR-T cells and justified the advancement of MC10029 CAR-T cells into our other experiments.

FIGS. 18A-18F show in vitro cytotoxicity of MC10029 CAR-T cells against CD-19 deficient malignant B-cell tumor lines. Three cell lines that are used to evaluate cytotoxicity of MC10029 CAR-T cells were tested for antigen expression. FIG. 18A) Using anti-BAFF-R-AF647 antibody, flow cytometry histograms show BAFF-R expression in Nalm-6, Z-138, or MEC-1. FIG. 18B) These cell lines were also evaluated for CD19 surface expression using anti-CD19-APC antibody. CD19 knock-out (KO) Nalm-6, Z-138, and MEC-1 variants were generated and evaluated for antigen surface expression. Flow cytometry histograms show BAFF-R expression in wild type Nalm-6, Z-138, or MEC-1 cell lines, as well as their CD19-deficient counterparts. FIGS. 18C and 18E) Flow cytometry plots show the functional potency of CAR-T cells against CD19-deficient tumor cells by the surface expression CD107a in a degranulation assay. Non-CAR-T cells, MC10029 CAR-T cells, or CD19 CAR-T cells were generated from the same donor and incubated with CD19-deficient Z-138 (FIG. 18C) or CD19-deficient MEC-1 (FIG. 18E) cells at an E:T ratio of 2:1 to characterize the cytotoxicity of MC10029 CAR-T cells against these CD19-deficient tumor cells. FIGS. 18D and 18F) Granzyme B ELISA shows functional potency of MC10029 CAR-T cells against CD19-deficient tumor cells. Non-CAR-T cells, MC10029 CAR-T cells, or CD19 CAR-T cells were co-incubated with CD19-deficient Z-138 (FIG. 18D) or CD19-deficient MEC-1 (FIG. 18F) cells at an E:T ratio of 4:1 for 72 hours when the supernatants were harvested for subsequent ELISA. Graphed data are means of quadruplicate sampling. The data are representative of three independent experiments.

FIGS. 19A-19D show activity of MC10029 CAR-T cells against primary CLL tumor cells. FIG. 19A) Basic demographic data for the nine B-CLL subjects evaluated with MC10029 CAR-T cells. FIG. 19B) Subject PBMCs were enriched for CLL tumor cells, and the removal of endogenous T cells was confirmed in these six samples by characterizing the CD3 positive T cells in the original PBMC samples (top panels) and enriched B cell population (bottom panels). FIG. 19C) Statistical analysis of degranulation data in FIG. 9B shows statistical significance in cytotoxicity against primary CLL tumor cells between MC10029 CAR-T cells and non-CAR-T cells (* p<0.05), while no difference was noted between Donor A and Donor B. FIG. 19D) Release of multiple granule proteins/cytokines from MC10029 CAR-T cells incubated with primary CLL tumor cells isolated from selected subjects. There was a significant increase observed in the release of granule proteins in CAR-T cell groups compared to the non-CAR-T cell groups. (**p<0.01).

FIGS. 20A-20F show analysis of CD107a degranulation assays. FIG. 20A) Statistical analysis of degranulation data of MC10029 CD4 CAR-T cells in FIG. 4A. FIG. 20B) Statistical analysis of degranulation data of MC10029 CD8 CAR-T cells in FIG. 4B. FIG. 20C) Statistical analysis of degranulation data of MC10029 CAR-T cells against CD19 KO Nalm-6 cells in FIG. 6A. FIG. 20D) Statistical analysis of degranulation data of MC10029 CAR-T cells against Z-138 cells in FIG. 7A. FIG. 20E) Statistical analysis of degranulation data of MC10029 CAR-T cells against MEC-1 cells in FIG. 8A. FIG. 20F) Statistical analysis of degranulation data of three production batches of MC10029 CAR-T cells in FIG. 16B. (** p<0.01; ns: no significance).

FIGS. 21A-21C show Antigen specific cytotoxicity of patient-derived MC10029 CAR T-cells against autologous B cells. FIG. 21A) The enrichment process of B cells from the peripheral blood of a representative patient efficiently depletes T cells (CD3 positive cells), resulting in a significant enrichment of CD20-positive B cells (95.9%). These enriched B cells exhibit a robust positive signal for BAFF-R expression (as shown in the top histogram). Enriched B cells from a healthy donor served as a positive control for T and B cell immunostaining. FIG. 21B) Antigen-specific cytotoxicity was assessed by measuring the surface expression of CD107a as a marker of degranulation. Isolated autologous B cells from a representative patient activated the patient-derived MC10029 CAR T-cells (MC10029 CAR-T), resulting in degranulation. A pair of BAFF-R-positive Nalm-6 and BAFF-R-deficient Nalm-6 cells (Nalm-6 BAFF-R KO) were included to validate the antigen-specific functionality of the CAR T-cells. FIG. 21C) Incubation of MC10029 CAR T-cells with autologous B cells expressing BAFF-R resulted in a significant release of the cytolytic protein granzyme B, in comparison to non-CAR T-cells (P=0.0123). The data were plotted as mean±SEM of triplicate sampling and analyzed using the student T test (* P<0.05; ** P<0.01; ns=not significant).

FIG. 22 shows a schematic diagram of an exemplary method for generating CAR T-cells from patient blood samples. (1) Blood is collected from sensitized kidney transplant patient, PBMCs are isolated. (2) B cells are enriched using the EasySep™ Direct Human B cell isolation kit, and surface marker characterization occurs to confirm purity and BAFF-R expression (3). (4) T cells are isolated using the Pan T cell isolation kit and transduced with MC10029 CAR lentivirus (5). (6) The resulting MC10029 CAR T-cells are expanded, characterized, and subjected to a complement of product release assays (7). (8) A second blood collection occurred at least 2 weeks after the first collection to isolate B cells. (9) B cells are enriched using the EasySep™ Human B cell isolation kit. (10) The MC10029 CAR T-cells from the patient are incubated with their autologous B cells. (11) Antigen specific cytotoxicity is determined using flow cytometry (CD107a degranulation assay) and by ELISA (cytokine release assay).

FIG. 23 shows confirmation of antigen-specific functionality of patient-derived MC10029 CAR T-cells from Patients 1-6. Patient-derived MC10029 CAR T-cells were stimulated with BAFF-R positive Nalm-6 cells, resulting in degranulation. Degranulation was measured by evaluating the surface expression of CD107a, a well-established marker of degranulation. BAFF-R-deficient Nalm-6 cells (Nalm-6 BAFF-R KO) were used as an antigen-negative control to confirm the antigen-specific functionality of the CAR T-cells.

FIGS. 24A-24B show antigen-specific cytotoxicity of MC10029 CAR T-cells derived from a sensitized patient who failed lung transplant showed activity against autologous B cells. FIG. 24A) Enriched autologous B cells activated the patient-derived MC10029 CAR T-cells (MC10029 CAR-T), resulting in degranulation. BAFF-R-positive Nalm-6 and BAFF-R-deficient Nalm-6 cells (Nalm-6 BAFF-R KO) were included as controls. FIG. 24B) A significant release of granzyme B was detected when MC10029 CAR T-cells from Patient 10 were incubated with autologous B cells or BAFF-R positive Nalm-6 cells but not when incubated with non-CAR-T cells. (*** P<0.001; ns=not significant).

FIG. 25 shows antigen-specific cytotoxicity of patient-derived MC10029 CAR T-cells from Patient 7. Patient-derived MC10029 CAR T-cells were stimulated with BAFF-R positive Nalm-6 cells, resulting in degranulation. Degranulation was measured by evaluating the surface expression of CD107a, a well-established marker of degranulation. BAFF-R-deficient Nalm-6 cells (Nalm-6 BAFF-R KO) were used as an antigen-negative control to confirm the antigen-specific functionality of the CAR T-cells.

FIGS. 26A-26B show direct killing of B cells by sensitized kidney transplant patient-derived MC10029 CAR T-cells. FIG. 26A) A direct killing assay that utilized B cells as the target cells and MC10029 CAR T-cells derived from a healthy donor was developed. non-CAR T-cells or MC10029 CAR T-cells were co-incubated with freshly isolated autologous B cells at an E:T ratio of 10:1 for 1 hour. Cells were then evaluated by flow cytometry to quantify the live CD20+B cells. non-CAR T-cells served as a negative control. FIG. 26B) MC10029 CAR T-cells obtained from a sensitized kidney transplant patient (Patient 5) demonstrated the ability to induce cell killing in B cells freshly isolated from PBMCs of a healthy donor.

### DETAILED DESCRIPTION

This document provides binders (e.g., antibodies, antigen binding fragments, antibody domains, CARs, cell engagers, and ADCs) that bind (e.g., specifically bind) to a BAFF-R polypeptide (e.g., a human BAFF-R polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the BAFF-R amino acid set forth in SEQ ID NO:42 (see, e.g., Example 2).

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Färber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having a length of five to ten amino acids or a polypeptide linker as set forth in Example 10). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having a length of eight to 18 amino acid residues or a polypeptide linker as set forth in Example 10). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in Example 7, Example 8, and Example 9.

An antibody provided herein can include the CDRs as described herein (e.g., an antibody including a heavy chain variable domain having the CDRs of SEQ ID NOs:1-3 and a light chain variable domain having the CDRs of SEQ ID NOs:9-11) and can be configured to be a human antibody, a humanized antibody, or a chimeric antibody. In some cases, an antibody provided herein can include the CDRs as described herein (e.g., an antibody including a heavy chain variable domain having the CDRs of SEQ ID NOs:1-3 and a light chain variable domain having the CDRs of SEQ ID NOs:9-11) and can be a monoclonal antibody. In some cases, an antibody provided herein can include the CDRs as described herein (e.g., an antibody including a heavy chain variable domain having the CDRs of SEQ ID NOs:1-3 and a light chain variable domain having the CDRs of SEQ ID NOs:9-11) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., an antigen binding fragment including a heavy chain variable domain having the CDRs of SEQ ID NOs:1-3 and a light chain variable domain having the CDRs of SEQ ID NOs:9-11) and can be configured to be a human antigen binding fragment, a humanized antigen binding fragment, or a chimeric antigen binding fragment. In some cases, an antigen binding fragment provided herein can include the CDRs as described herein (e.g., an antigen binding fragment including a heavy chain variable domain having the CDRs of SEQ ID NOs:1-3 and a light chain variable domain having the CDRs of SEQ ID NOs:9-11) and can be a monoclonal antigen binding fragment. In some cases, an antigen binding fragment provided herein can include the CDRs as described herein (e.g., an antigen binding fragment including a heavy chain variable domain having the CDRs of SEQ ID NOs:1-3 and a light chain variable domain having the CDRs of SEQ ID NOs:9-11) and can be configured as a Fab antibody. In some cases, a Fab antibody can include a hinge sequence for disulfide bonding between heavy and light chains of the Fab.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., an antibody domain including a heavy chain variable domain having the CDRs of SEQ ID NOs:1-3 or a light chain variable domain having the CDRs of SEQ ID NOs:9-11) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., an antibody domain including a heavy chain variable domain having the CDRs of SEQ ID NOs:1-3 or a light chain variable domain having the CDRs of SEQ ID NOs:9-11) and can be a monoclonal antibody domain. In some cases, an antibody domain provided herein can include the CDRs as described herein (e.g., an antibody domain including a heavy chain variable domain having the CDRs of SEQ ID NOs:1-3 or a light chain variable domain having the CDRs of SEQ ID NOs:9-11) and can be engineered as a single VH domain or a single VL domain.

An anti-BAFF-R antibody, anti-BAFF-R antigen binding fragment, or anti-BAFF-R antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁- and IgM₂-types. In some cases, an antibody provided herein (e.g., an anti-BAFF-R antibody) can be a scFv antibody. In some cases, an antigen binding fragment provided herein (e.g., an anti-BAFF-R antibody fragment) can be a Fab. In some cases, an antibody provided herein (e.g., an anti-BAFF-R antibody) can be a fully intact antibody having the structure set forth in Example 6. In some cases, an antibody domain provided herein (e.g., an anti-BAFF-R antibody domain) can be a VH domain.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a BAFF-R polypeptide (e.g., a human BAFF-R polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a BAFF-R polypeptide (e.g., a human BAFF-R polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11). In some cases, an antigen binding domain of a CAR-T targeting a BAFF-R polypeptide can be designed to include a VH domain described herein or a scFv antibody described herein.

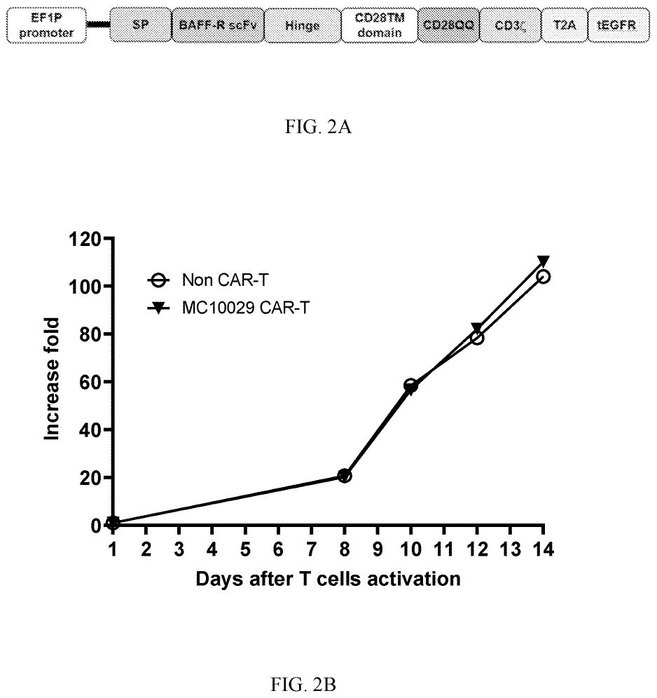

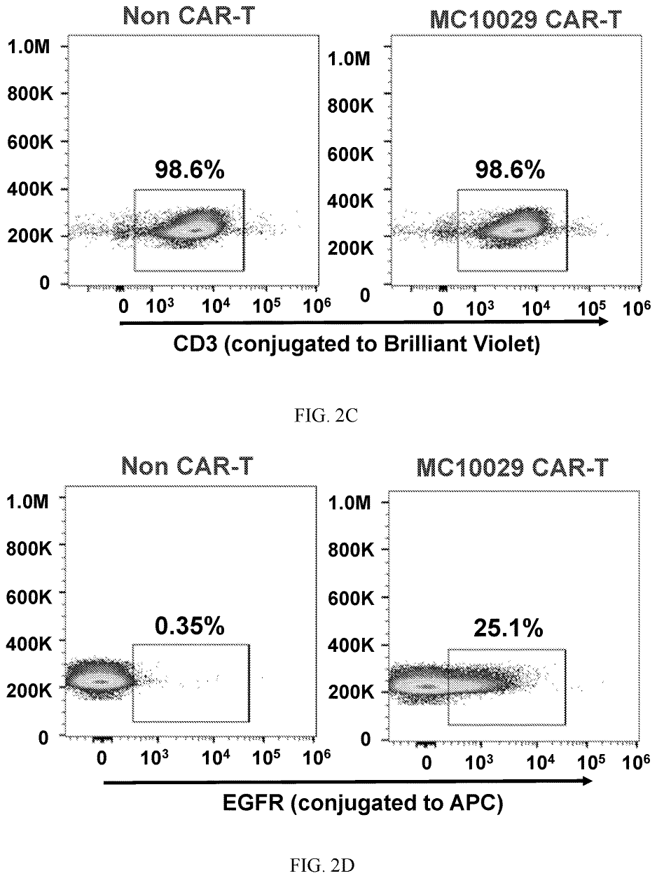

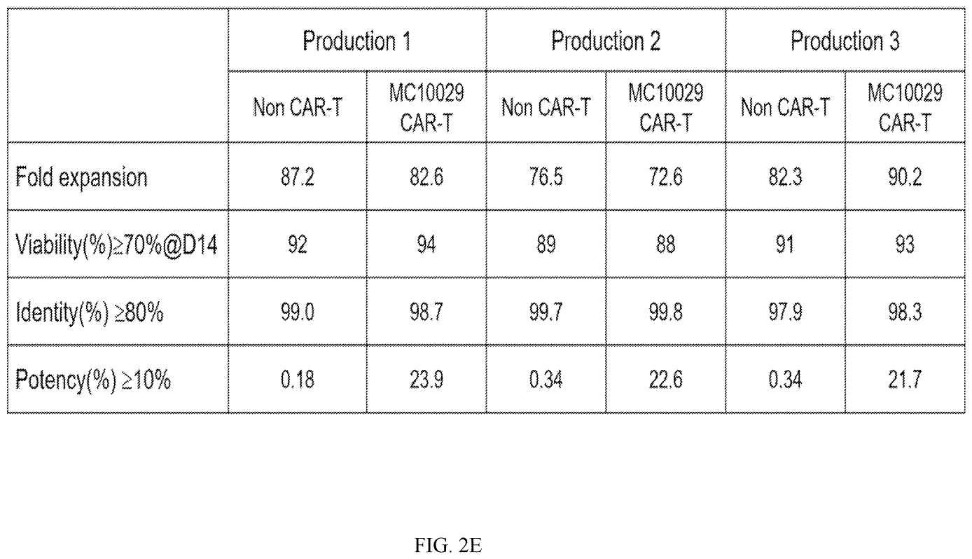

Examples of CAR structures that can be used to make a CAR provided herein include, without limitation, those set forth in FIG. 2A.

In some cases, a CAR provided herein can be designed to include a signal peptide. Any appropriate signal peptide can be used to design a CAR described herein. In some cases, a signal peptide that can be used to make a CAR described herein can be derived from a human polypeptide. Examples of signal peptides that can be used to make a CAR described herein include without limitation, GMCSF-derived signal peptides (e.g., a human GMCSF-derived signal peptide), CD3-derived signal peptides, CD4-derived signal peptides, and CD8-derived signal peptides. In some cases, a CAR provided herein can be designed to include a signal peptide that comprises, consists essentially of, or consists of one of the amino acid sequences set forth in Example 11. In some cases, a CAR provided herein can be designed to include a signal peptide that comprises, consists essentially of, or consists of one of the amino acid sequences set forth in Example 11 with one, two, three, four, five, six, seven, eight, nine, or ten amino acid deletions, additions, substitutions, or combinations thereof. In some cases, a CAR provided herein can be designed to include a signal peptide that comprises, consists essentially of, or consists of one of the amino acid sequences set forth in Example 11 with two or less, three or less, four or less, five or less, six or less, seven or less, eight or less, nine or less, or ten or less amino acid deletions, additions, substitutions, or combinations thereof.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG4-derived hinge such as a human IgG4-derived hinge), CD8-derived hinges (e.g., partial CD8 extracellular domains), and CD28-derived hinges (e.g., partial CD28 extracellular domains). A CAR provided herein can be designed to include a hinge of any appropriate length. For example, a CAR provided herein can be designed to include a hinge that is from about 3 to about 250 (e.g., from about 3 to about 200, from about 3 to about 150, from about 3 to about 100, from about 3 to about 50, from about 5 to about 250, from about 25 to about 250, from about 50 to about 250, from about 75 to about 250, from about 100 to about 250, from about 150 to about 250, from about 5 to about 200, from about 10 to about 150, from about 20 to about 100, from about 25 to about 75, from about 10 to about 40, from about 20 to about 50, from about 30 to about 60, from about 50 to about 100, or from about 80 to about 120) amino acid residues in length. In some cases, a linker sequence can be used as a hinge to make a CAR described herein. For example, any one of the linker sequences set forth in Example 10 can be used as a hinge of a CAR described herein. In some cases, a CAR provided herein can be designed to include a hinge that comprises, consists essentially of, or consists of one of the amino acid sequences set forth in Example 10 or Example 12. In some cases, a CAR provided herein can be designed to include a hinge that comprises, consists essentially of, or consists of one of the amino acid sequences set forth in Example 10 or Example 12 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in Example 10 or Example 12 with two or less, three or less, four or less, five or less, six or less, seven or less, eight or less, nine or less, or ten or less amino acid deletions, additions, substitutions, or combinations thereof.

A CAR provided herein can be designed to include any appropriate transmembrane domain. Examples of transmembrane domains that can be used to make a CAR described herein include, without limitation, CD28 transmembrane domains, CD4 transmembrane domains, CD8 transmembrane domains, CD3 zeta transmembrane domains, and ICOS transmembrane domains. In some cases, a CAR provided herein can be designed to include a transmembrane domain that comprises, consists essentially of, or consists of one of the amino acid sequences set forth in Example 13. In some cases, a CAR provided herein can be designed to include a transmembrane domain that comprises, consists essentially of, or consists of one of the amino acid sequences set forth in Example 13 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in Example 13 with two or less, three or less, four or less, five or less, six or less, seven or less, eight or less, nine or less, or ten or less amino acid deletions, additions, substitutions, or combinations thereof.

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD28 intracellular signaling domains, 4-1BB intracellular signaling domains, OX40 intracellular signaling domains, and ICOS intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in Example 14. In some cases, a CAR provided herein can be designed to include at least one intracellular signaling domain that comprises, consists essentially of, or consists of one of the amino acid sequences set forth in Example 14 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in Example 14 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting a BAFF-R polypeptide can be designed to include an scFv having a heavy chain variable domain comprising SEQ ID NO: 1, SEQ ID NO:2, and SEQ ID NO:3, followed by a linker such as a linker set forth in Example 10, followed by a light chain variable domain comprising SEQ ID NO:9, SEQ ID NO:10, and SEQ ID NO:11, followed by a hinge such as a hinge/linker set forth in Example 10 or Example 12 (e.g., an IgG4-derived hinge), followed by a transmembrane domain such as a transmembrane domain set forth in Example 13 (e.g., a human CD28 transmembrane domain), followed by one or more intracellular signaling domains such as one or more intracellular signaling domain set forth in Example 14 (e.g., a human CD28 intracellular signaling domain followed by a human CD3ζ intracellular signaling domain). For example, a CAR targeting a BAFF-R polypeptide can be designed to include an scFv having a heavy chain variable domain comprising SEQ ID NO: 1, SEQ ID NO:2, and SEQ ID NO:3, followed by SEQ ID NO:25, followed by a light chain variable domain comprising SEQ ID NO:9, SEQ ID NO: 10, and SEQ ID NO: 11, followed by SEQ ID NO:30, followed by SEQ ID NO:32, followed by SEQ ID NO:34, followed by SEQ ID NO:36, followed by SEQ ID NO:38.

In some cases, a CAR targeting a BAFF-R polypeptide can be designed to include an scFv having a heavy chain variable domain comprising SEQ ID NO:8, followed by a linker such as a linker set forth in Example 10, followed by a light chain variable domain comprising SEQ ID NO: 16, followed by a hinge such as a hinge/linker set forth in Example 10 or Example 12 (e.g., an IgG4-derived hinge), followed by a transmembrane domain such as a transmembrane domain set forth in Example 13 (e.g., a human CD28 transmembrane domain), followed by one or more intracellular signaling domains such as one or more intracellular signaling domain set forth in Example 14 (e.g., a human CD28 intracellular signaling domain followed by a human CD3ζ intracellular signaling domain). For example, a CAR targeting a BAFF-R polypeptide can be designed to include an scFv having a heavy chain variable domain comprising SEQ ID NO:8, followed by SEQ ID NO: 25, followed by a light chain variable domain comprising SEQ ID NO: 16, followed by SEQ ID NO:30, followed by SEQ ID NO:32, followed by SEQ ID NO:34, followed by SEQ ID NO:36, followed by SEQ ID NO:38.

In some cases, a CAR targeting a BAFF-R polypeptide can be designed to include an scFv having a light chain variable domain comprising SEQ ID NO:9, SEQ ID NO: 10, and SEQ ID NO: 11, followed by a linker such as a linker set forth in Example 10, followed by a heavy chain variable domain comprising SEQ ID NO:1, SEQ ID NO:2, and SEQ ID NO:3, followed by a hinge such as a hinge/linker set forth in Example 10 or Example 12 (e.g., an IgG4-derived hinge), followed by a transmembrane domain such as a transmembrane domain set forth in Example 13 (e.g., a human CD28 transmembrane domain), followed by one or more intracellular signaling domains such as one or more intracellular signaling domain set forth in Example 14 (e.g., a human CD28 intracellular signaling domain followed by a human CD3ζ intracellular signaling domain). For example, a CAR targeting a BAFF-R polypeptide can be designed to include an scFv having a light chain variable domain comprising SEQ ID NO:9, SEQ ID NO:10, and SEQ ID NO: 11, followed by SEQ ID NO: 25, followed by a heavy chain variable domain comprising SEQ ID NO: 1, SEQ ID NO:2, and SEQ ID NO:3, followed by SEQ ID NO:30, followed by SEQ ID NO:32, followed by SEQ ID NO:34, followed by SEQ ID NO:36, followed by SEQ ID NO:38 (see, e.g., Example 16).

In some cases, a CAR targeting a BAFF-R polypeptide can be designed to include an scFv having a light chain variable domain comprising SEQ ID NO: 16, followed by a linker such as a linker set forth in Example 10, followed by a heavy chain variable domain comprising SEQ ID NO:8, followed by a hinge such as a hinge/linker set forth in Example 10 or Example 12 (e.g., an IgG4-derived hinge), followed by a transmembrane domain such as a transmembrane domain set forth in Example 13 (e.g., a human CD28 transmembrane domain), followed by one or more intracellular signaling domains such as one or more intracellular signaling domain set forth in Example 14 (e.g., a human CD28 intracellular signaling domain followed by a human CD3ζ intracellular signaling domain). For example, a CAR targeting a BAFF-R polypeptide can be designed to include an scFv having a light chain variable domain comprising SEQ ID NO: 16, followed by SEQ ID NO: 25, followed by a heavy chain variable domain comprising SEQ ID NO:8, followed by SEQ ID NO:30, followed by SEQ ID NO:32, followed by SEQ ID NO:34, followed by SEQ ID NO:36, followed by SEQ ID NO:38 (see, e.g., Example 16).

In some cases, a CAR provided herein can include (e.g., can be designed to include) one or more additional components. Examples of additional components that can be included in a CAR provided herein include, without limitation, detectable markers, suicide switches, and surface markers that aid in enrichment. When a CAR provided herein includes a detectable marker, the detectable marker can be any appropriate detectable marker. Examples of detectable markers that can be included in a CAR provided herein include, without limitation, non-functional polypeptides (e.g., truncated epidermal growth factor receptor (tEGFR) polypeptides), bioluminescent polypeptides (e.g., luciferase polypeptides), fluorescent polypeptides (e.g., green fluorescent polypeptides (GFPs)), and ribosomal skipping polypeptides (e.g., T2A polypeptides). For example, a CAR provided herein can include a tEGFR polypeptide that comprises, consists essentially of, or consists of the amino acid sequence set forth in SEQ ID NO:38 (see, e.g., Example 15).

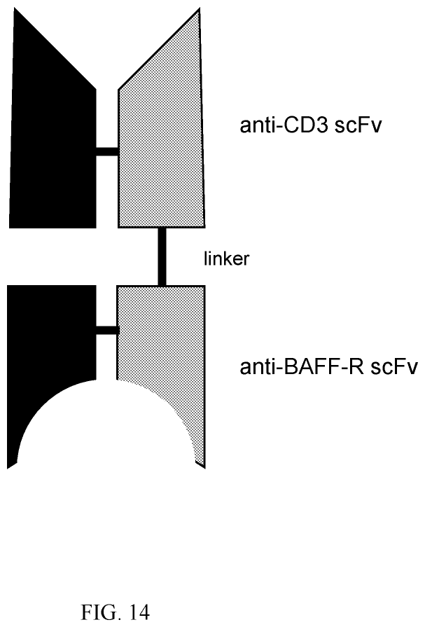

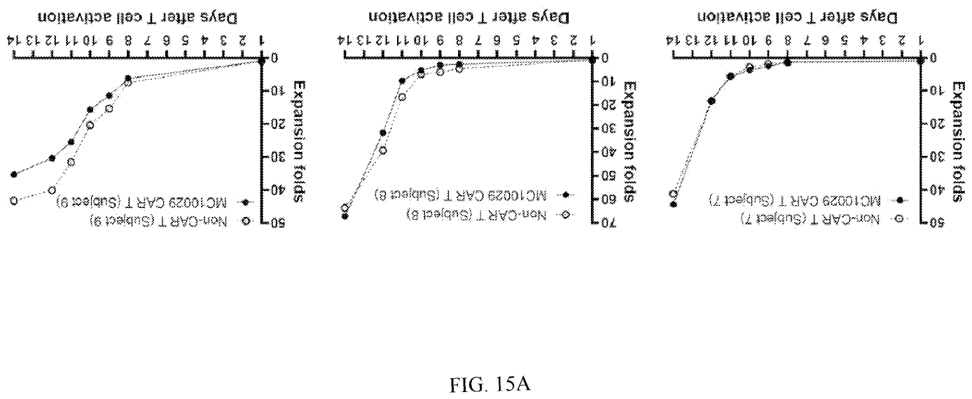

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a BAFF-R polypeptide (e.g., a human BAFF-R polypeptide) and at least one antigen binding domain having the ability to bind to an antigen expressed on the surface of a cell (e.g., a T cell or an NK cell). In some cases, a cell engager described herein can link a BAFF-R⁺ cell (e.g., a BAFF-R⁺ cancer cell) to another cell (e.g., a T cell or an NK cell) via the two or more antigen binding domains of the cell engager. An example of a cell engager structure of cell engagers provided herein includes, without limitation, the structure set forth in FIG. 14. In some cases, the anti-CD3 scFv depicted in FIG. 14 can be replaced with a different antigen binding domain having the ability to bind to an antigen expressed on the surface of a cell (e.g., a T cell or an NK cell).

When a cell engager includes an antigen binding domain having the ability to bind to a BAFF-R polypeptide (e.g., a human BAFF-R polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to a BAFF-R polypeptide (e.g., a human BAFF-R polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to a BAFF-R polypeptide (e.g., a human BAFF-R polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a BAFF-R polypeptide (e.g., a human BAFF-R polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11). In some cases, an antigen binding domain of a cell engager targeting a BAFF-R polypeptide can be designed to include a VH domain described herein or a scFv or Fab antibody described herein. In some cases, an antigen binding domain of a CAR described herein that has the ability to bind to a BAFF-R polypeptide (e.g., a human BAFF-R polypeptide) can be used as an antigen binding domain of a cell engager that targets BAFF-R⁺ cells.

As described herein, a cell engager can be designed to include at least one antigen binding domain having the ability to bind to a BAFF-R polypeptide (e.g., a human BAFF-R polypeptide) and at least one other antigen binding domain. That at least one other antigen binding domain can have the ability to bind to any appropriate antigen expressed on the surface of a cell. For example, when designing a cell engager such as a BiTE to link a BAFF-Ri cell and a T cell, the cell engager can include an antigen binding domain having the ability to bind to a BAFF-R polypeptide (e.g., a human BAFF-R polypeptide) and an antigen binding domain having the ability to bind to a polypeptide expressed on the surface of a T cell. Examples of polypeptides expressed on the surface of a T cell that can be targeted by an antigen binding domain of a cell engager provided herein include, without limitation, CD3 polypeptides and 4-1BB polypeptides. Examples of antigen binding domains having the ability to bind to a polypeptide expressed on the surface of a T cell that can be used to make a cell engager provided herein (e.g., a BiTE) include, without limitation, anti-CD3 scFvs, anti-CD3 VH domains, anti-4-1BB scFvs, and anti-4-1BB VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

When designing a cell engager such as a BiKE or a TriKE to link a BAFF-Ri cell and an NK cell, the cell engager can include an antigen binding domain having the ability to bind to a BAFF-R polypeptide (e.g., a human BAFF-R polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16a polypeptides, NKG2A polypeptides, NKG2D polypeptides, NKp30 polypeptides, NKp44 polypeptides, NKp46 polypeptides, and CRTAM polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-CD16a VH domains, anti-NKG2A scFvs, anti-NKG2A VH domains, anti-NKG2D scFvs, anti-NKG2D VH domains, anti-NKp30 scFvs, anti-NKp30 VH domains, anti-NKp44 scFvs, anti-NKp44 VH domains, anti-NKp46 scFvs, anti-NKp46 VH domains, anti-CRTAM scFvs, and anti-CRTAM VH domains.

In some cases, a cell engager provided herein (e.g., a BiTE) can be designed to include a linker located between each antigen binding domain. Any appropriate linker can be used to design a cell engager provided herein. Examples of linkers that can be used to make a cell engager described herein include, without limitation, the linker sequences set forth in Example 10. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a hinge of a CAR described herein can be used as a linker to make a cell engager described herein. For example, any one of the sequences set forth in Example 12 can be used as a linker of a cell engager described herein.

In some cases, a cell engager provided herein can be designed to include a linker that comprises, consists essentially of, or consists of one of the amino acid sequences set forth in Example 10 or Example 12. In some cases, a cell engager provided herein can be designed to include a linker that comprises, consists essentially of, or consists of one of the amino acid sequences set forth in Example 10 or Example 12 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in Example 10 or Example 12 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting a BAFF-R polypeptide can be designed to include an scFv having a heavy chain variable domain comprising SEQ ID NO: 1, SEQ ID NO:2, and SEQ ID NO:3, followed by a linker such as a linker set forth in Example 10, followed by a light chain variable domain comprising SEQ ID NO:9, SEQ ID NO: 10, and SEQ ID NO: 11, followed by a linker such as a hinge/linker set forth in Example 10 or Example 12, followed by an antigen binding domain having the ability to bind to a polypeptide expressed on the surface of a T cell (e.g., an anti-human CD3 scFv).

In some cases, a cell engager (e.g., a BiTE) targeting a BAFF-R polypeptide can be designed to include an scFv having a light chain variable domain comprising SEQ ID NO:9, SEQ ID NO: 10, and SEQ ID NO: 11, followed by a linker such as a linker set forth in Example 10, followed by a heavy chain variable domain comprising SEQ ID NO: 1, SEQ ID NO:2, and SEQ ID NO:3, followed by a linker such as a hinge/linker set forth in Example 10 or Example 12 (e.g., SEQ ID NO:30), followed by an antigen binding domain having the ability to bind to a polypeptide expressed on the surface of a T cell (e.g., an anti-human CD3 scFv).

In some cases, a cell engager (e.g., a BiTE) targeting a BAFF-R polypeptide can be designed to include an scFv having a heavy chain variable domain comprising SEQ ID NO:8, followed by a linker such as a linker set forth in Example 10, followed by a light chain variable domain comprising SEQ ID NO: 16, followed by a linker such as a hinge/linker set forth in Example 10 or Example 12 (e.g., SEQ ID NO:30), followed by an antigen binding domain having the ability to bind to a polypeptide expressed on the surface of a T cell (e.g., an anti-human CD3 scFv).

In some cases, a cell engager (e.g., a BiTE) targeting a BAFF-R polypeptide can be designed to include an scFv having a light chain variable domain comprising SEQ ID NO: 16, followed by a linker such as a linker set forth in Example 10, followed by a heavy chain variable domain comprising SEQ ID NO:8, followed by a linker such as a hinge/linker set forth in Example 10 or Example 12 (e.g., SEQ ID NO:30), followed by an antigen binding domain having the ability to bind to a polypeptide expressed on the surface of a T cell (e.g., an anti-human CD3 scFv).

In some cases, a cell engager (e.g., a BiKE or a TriKE) targeting a BAFF-R polypeptide can be designed to include an scFv having a heavy chain variable domain comprising SEQ ID NO: 1, SEQ ID NO:2, and SEQ ID NO:3, followed by a linker such as a linker set forth in Example 10, followed by a light chain variable domain comprising SEQ ID NO:9, SEQ ID NO:10, and SEQ ID NO: 11, followed by a linker such as a hinge/linker set forth in Example 10 or Example 12 (e.g., SEQ ID NO:30), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a BAFF-R polypeptide can be designed to include an scFv having a light chain variable domain comprising SEQ ID NO:9, SEQ ID NO: 10, and SEQ ID NO: 11, followed by a linker such as a linker set forth in Example 10, followed by a heavy chain variable domain comprising SEQ ID NO: 1, SEQ ID NO:2, and SEQ ID NO:3, followed by a linker such as a hinge/linker set forth in Example 10 or Example 12 (e.g., SEQ ID NO:30), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a BAFF-R polypeptide can be designed to include an scFv having a heavy chain variable domain comprising SEQ ID NO:8, followed by a linker such as a linker set forth in Example 10, followed by a light chain variable domain comprising SEQ ID NO:16, followed by a linker such as a hinge/linker set forth in Example 10 or Example 12 (e.g., SEQ ID NO:30), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a BAFF-R polypeptide can be designed to include an scFv having a light chain variable domain comprising SEQ ID NO: 16, followed by a linker such as a linker set forth in Example 10, followed by a heavy chain variable domain comprising SEQ ID NO:8, followed by a linker such as a hinge/linker set forth in Example 10 or Example 12 (e.g., SEQ ID NO:30), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a BAFF-R polypeptide can be designed to include an IgG (e.g., IgG1) configuration having (a) a heavy chain comprising, consisting essentially of, or consisting of a heavy chain variable domain comprising SEQ ID NO: 1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting a BAFF-R polypeptide can be designed to include an IgG (e.g., IgG1) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO: 16, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting a BAFF-R polypeptide can be designed to include an IgG (e.g., IgG1) configuration having (a) a heavy chain comprising, consisting essentially of, or consisting of a heavy chain variable domain comprising SEQ ID NO: 1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting a BAFF-R polypeptide can be designed to include an IgG (e.g., IgG1) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO: 16, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In one embodiment, a binder (e.g., an antibody, antigen binding fragment, antibody domain, a CAR, a cell engager, and/or an ADC) provided herein having the ability to bind to a BAFF-R polypeptide (e.g., a human BAFF-R polypeptide) can include (i) a heavy chain variable domain having a CDR1 having the amino acid sequence set forth in SEQ ID NO:1 (or a variant of SEQ ID NO:1 with one, two, three, or four amino acid modifications), a CDR2 having the amino acid sequence set forth in SEQ ID NO:2 (or a variant of SEQ ID NO:2 with one, two, three, or four amino acid modifications), and a CDR3 having the amino acid sequence set forth in SEQ ID NO:3 (or a variant of SEQ ID NO:3 with one, two, three, or four amino acid modifications); and/or (ii) a light chain variable domain having a CDR1 having the amino acid sequence set forth in SEQ ID NO:9 (or a variant of SEQ ID NO:9 with one, two, three, or four amino acid modifications), a CDR2 having the amino acid sequence set forth in SEQ ID NO:10 (or a variant of SEQ ID NO: 10 with one, two, three, or four amino acid modifications), and a CDR3 having the amino acid sequence set forth SEQ ID NO: 11 (or a variant of SEQ ID NO: 11 with one, two, three, or four amino acid modifications). Examples of such an antigen binding fragments having these CDRs and the ability to bind to a BAFF-R polypeptide (e.g., a human BAFF-R polypeptide) include, without limitation, the variable domains set forth in Example 3 and the Fabs set forth in Example 4.

In some cases, a binder (e.g., an antibody, antigen binding fragment, antibody domain, a CAR, a cell engager, and/or an ADC) provided herein having the ability to bind to a BAFF-R polypeptide (e.g., a human BAFF-R polypeptide) and (a) a heavy chain variable domain having a CDR1 having the amino acid sequence set forth in SEQ ID NO:1 (or a variant of SEQ ID NO:1 with one, two, three, or four amino acid modifications), a CDR2 having the amino acid sequence set forth in SEQ ID NO:2 (or a variant of SEQ ID NO:2 with one, two, three, or four amino acid modifications), and a CDR3 having the amino acid sequence set forth in SEQ ID NO:3 (or a variant of SEQ ID NO:3 with one, two, three, or four amino acid modifications) and/or (b) a light chain variable domain having a CDR1 having the amino acid sequence set forth in SEQ ID NO:9 (or a variant of SEQ ID NO:9 with one, two, three, or four amino acid modifications), a CDR2 having the amino acid sequence set forth in SEQ ID NO:10 (or a variant of SEQ ID NO: 10 with one, two, three, or four amino acid modifications), and a CDR3 having the amino acid sequence set forth SEQ ID NO: 11 (or a variant of SEQ ID NO: 11 with one, two, three, or four amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO: 12 (or a variant of SEQ ID NO: 12 with one, two, three, four, five, six, seven, eight, nine, ten, or more amino acid modifications), a framework region 2 having the amino acid sequence set forth in SEQ ID NO: 13 (or a variant of SEQ ID NO: 13 with one, two, three, four, five, six, seven, eight, nine, ten, or more amino acid modifications), a framework region 3 having the amino acid sequence set forth in SEQ ID NO: 14 (or a variant of SEQ ID NO: 14 with one, two, three, four, five, six, seven, eight, nine, ten, or more amino acid modifications), and a framework region 4 having the amino acid sequence set forth in SEQ ID NO: 15 (or a variant of SEQ ID NO: 15 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in Example 3 can be designed to include framework regions as set forth in Example 3 or can be designed to include one or more framework regions from another antibody, antibody fragment, or antibody domain. For example, a Fab can be designed to include the six CDRs set forth in Example 3 and the framework regions set forth in Example 3 except that one or more of the framework regions can be replaced with a framework region set forth in Example 8 or Example 9. In some cases, a scFv can be designed to include the six CDRs set forth in Example 3 and the framework regions set forth in Example 3. In some cases, a scFv can be designed to include the six CDRs set forth in Example 3 and the framework regions set forth in Example 3 except that one or more of the framework regions can be replaced with a framework region set forth in Example 8 or Example 9.

In some cases, a binder (e.g., an antibody, antigen binding fragment, antibody domain, a CAR, a cell engager, and/or an ADC) provided herein having the ability to bind to a BAFF-R polypeptide (e.g., a human BAFF-R polypeptide) can include (a) a heavy chain variable domain that includes an amino acid sequence having at least 85 percent identity to the amino acid sequence set forth in SEQ ID NO:8 and/or (b) a light chain variable domain that includes an amino acid sequence having at least 85 percent identity to the amino acid sequence set forth in SEQ ID NO:16. For example, a binder (e.g., an antibody, antigen binding fragment, antibody domain, a CAR, a cell engager, and/or an ADC) provided herein can include (a) a heavy chain variable domain that includes an amino acid sequence having at least 85, 86, 87, 88, 89, 90, 91, 92, 93, 94, 95, 96, 97, 98, or 99 percent identity to the amino acid sequence set forth in SEQ ID NO:8 and/or (b) a light chain variable domain that includes an amino acid sequence having at least 85, 86, 87, 88, 89, 90, 91, 92, 93, 94, 95, 96, 97, 98, or 99 percent identity to the amino acid sequence set forth in SEQ ID NO: 16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO: 16.

In some cases, a binder (e.g., an antibody, antigen binding fragment, antibody domain, a CAR, a cell engager, and/or an ADC) provided herein having the ability to bind to a BAFF-R polypeptide (e.g., a human BAFF-R polypeptide) can include (a) a heavy chain variable domain that includes an amino acid sequence having at least 85 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 85 percent identity to the amino acid sequence set forth in SEQ ID NO: 16, provided that the light chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 85, 86, 87, 88, 89, 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 85, 86, 87, 88, 89, 90, 91, 92, 93, 94, 95, 96, 97, 98, or 99 percent identity to the amino acid sequence set forth in SEQ ID NO: 16, provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a BAFF-R polypeptide (e.g., a human BAFF-R polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO: 16 or the amino acid set forth in SEQ ID NO: 16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a BAFF-R polypeptide (e.g., a human BAFF-R polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3, and can include a light chain variable domain having the amino acid sequence set forth in SEQ ID NO: 16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a BAFF-R polypeptide (e.g., a human BAFF-R polypeptide) can include (a) a heavy chain variable domain comprising (i) a CDR1 that comprises, consists essentially of, or consists of the amino acid sequence set forth in SEQ ID NO: 1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO: 10, and (iii) a CDR3 that comprises, consists essentially of, or consists of the amino acid sequence set forth in SEQ ID NO: 11. As used herein, a “CDR1 that consists essentially of the amino acid sequence set forth in SEQ ID NO: 1” is a CDR1 that has zero, one, or two amino acid substitutions within SEQ ID NO: 1, that has zero, one, two, three, four, or five amino acid residues directly preceding SEQ ID NO: 1, and/or that has zero, one, two, three, four, or five amino acid residues directly following SEQ ID NO: 1, provided that the binder (e.g., an antibody, antigen binding fragment, antibody domain, a CAR, a cell engager, and/or an ADC) maintains its basic ability to bind to a BAFF-R polypeptide (e.g., a human BAFF-R polypeptide).

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a BAFF-R polypeptide (e.g., a human BAFF-R polypeptide).

As used herein, a “CDR3 that consists essentially of the amino acid sequence set forth in SEQ ID NO:3” is a CDR3 that has zero, one, or two amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a BAFF-R polypeptide (e.g., a human BAFF-R polypeptide).

As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a BAFF-R polypeptide (e.g., a human BAFF-R polypeptide).

As used herein, a “CDR2 that consists essentially of the amino acid sequence set forth in SEQ ID NO:10” is a CDR2 that has zero, one, or two amino acid substitutions within SEQ ID NO: 10, that has zero, one, two, three, four, or five amino acid residues directly preceding SEQ ID NO:10, and/or that has zero, one, two, three, four, or five amino acid residues directly following SEQ ID NO: 10, provided that the binder (e.g., an antibody, antigen binding fragment, antibody domain, a CAR, a cell engager, and/or an ADC) maintains its basic ability to bind to a BAFF-R polypeptide (e.g., a human BAFF-R polypeptide).

As used herein, a “CDR3 that consists essentially of the amino acid sequence set forth in SEQ ID NO: 11” is a CDR3 that has zero, one, or two amino acid substitutions within SEQ ID NO: 11, that has zero, one, two, three, four, or five amino acid residues directly preceding SEQ ID NO: 11, and/or that has zero, one, two, three, four, or five amino acid residues directly following SEQ ID NO: 11, provided that the binder (e.g., an antibody, antigen binding fragment, antibody domain, a CAR, a cell engager, and/or an ADC) maintains its basic ability to bind to a BAFF-R polypeptide (e.g., a human BAFF-R polypeptide).

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:1-3 can be directly connected to a light chain variable domain having the CDRs of SEQ ID NOs:9-11, respectively, via a linker sequence. Examples of linker sequences that can be used to connect a heavy chain variable domain and a light chain variable domain to create a scFv include, without limitation, those linkers set forth in Example 10.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr Opin. Biotechnol. 3: 348-354 (1992). Both naturally occurring and non-naturally occurring amino acids (e.g., artificially-derivatized amino acids) can be used to generate an amino acid sequence variant provided herein.

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. Example 5 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a BAFF-R polypeptide (e.g., a human BAFF-R polypeptide). In some cases, a bispecific binder provided herein can be designed to bind to two different epitopes of the same BAFF-R polypeptide (e.g., a human BAFF-R polypeptide). In some cases, a bispecific binder provided herein can bind to a BAFF-R polypeptide (e.g., a human BAFF-R polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. In some cases, a polymer can be in the form of a polymeric nanoparticle. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, poly(lactic-co-glycolic acid) (PLGA), and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to or contained within a nanoparticle. For example, a binder (e.g., an antibody, antigen binding fragment, antibody domain, CAR, cell engager, and/or ADC) provided herein can be attached to or contained within a nanoparticle having carbodimide chemistry. For example, a binder (e.g., an antibody, antigen binding fragment, antibody domain, CAR, cell engager, and/or ADC) provided herein can be attached to or contained within a nanoparticle having maleimide chemistry. In some cases, a nanoparticle that is attached to or contains a binder (e.g., an antibody, antigen binding fragment, antibody domain, CAR, cell engager, and/or ADC) provided herein also can include chitosan, BSA, and/or biotin.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a BAFF-R polypeptide (e.g., a human BAFF-R polypeptide) and (b) at least one drug covalently linked directly or indirectly to that antigen binding domain. Any appropriate binder (e.g., an antibody, antigen binding fragment, and/or antibody domain) provided herein and having the ability to bind to a BAFF-R polypeptide (e.g., a human BAFF-R polypeptide) can be used as an antigen binding domain to make an ADC described herein. For example, any of the binders set forth in Example 3 and Example 4 can be used to make an ADC having the ability to bind to a BAFF-R polypeptide (e.g., a human BAFF-R polypeptide). Examples of drugs that can be used to make an ADC described herein include, without limitation, auristatins (e.g., monomethyl auristatin E (MMAE)), mertansine (DM-1), pyrrolobenzodiazepine (PBD) dimers, tubulysins, duocarmycins, and calicheamicins. Any appropriate ADC linker can be used to covalently attach one or more drugs to an antigen binding domain having the ability to bind to a BAFF-R polypeptide (e.g., a human BAFF-R polypeptide) to form an ADC provided herein. For example, cleavable or non-cleavable ADC linkers can be used to covalently attach one or more drugs to an antigen binding domain having the ability to bind to a BAFF-R polypeptide (e.g., a human BAFF-R polypeptide) to form an ADC provided herein. Examples of ADC linkers can be used to covalently attach one or more drugs to an antigen binding domain having the ability to bind to a BAFF-R polypeptide (e.g., a human BAFF-R polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a heavy chain variable domain such as a heavy chain variable domain as set forth in Example 3. In another example, an isolated nucleic acid molecule provided herein can include a nucleic acid sequence encoding a light chain variable domain such as a light chain variable domain as set forth in Example 3. In some cases, an isolated nucleic acid molecule provided herein can include a nucleic acid sequence encoding both (a) a heavy chain variable domain and (b) a light chain variable domain, with or without, encoding a linker polypeptide set forth in Example 10. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

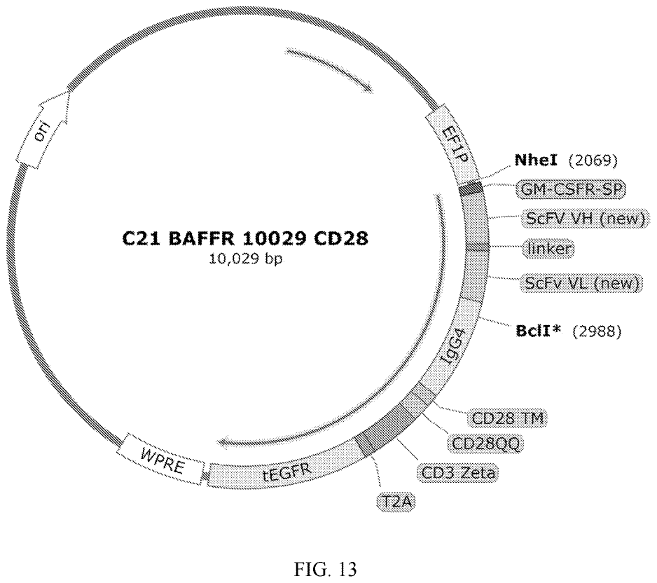

Examples of vectors that include a nucleic acid sequence encoding scFv or antibody domain (e.g., a VH domain) provided herein include, without limitation, those set forth in FIG. 13.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokaryotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, Chinese hamster ovary (CHO) cells, W138 BHK cells, COS-7 cells, 293FT cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a BAFF-R polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a BAFF-R polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer (e.g., one or more B-cell cancers) to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a BAFF-R polypeptide (e.g., a human BAFF-R polypeptide) can be formulated as a pharmaceutical composition for administration to a mammal (e.g., a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a BAFF-R polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a BAFF-R polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10′ to about 1×10¹², from about 1×10⁶ to about 1×10¹¹, or from about 1×10⁷ to about 1×10¹⁰). In some cases, a pharmaceutical composition containing one or more cells designed to express a CAR having the ability to bind to a BAFF-R polypeptide provided herein can be formulated as a dosage form with from about 25 million anti-BAFF-R CAR⁺ cells to about 400 million anti-BAFF-R CAR⁺ cells (e.g., from about 25 million to about 350 million, from about 25 million to about 300 million, from about 25 million to about 250 million, from about 25 million to about 200 million, from about 25 million to about 150 million, from about 25 million to about 100 million, from about 25 million to about 50 million, from about 50 million to about 400 million, from about 100 million to about 400 million, from about 150 million to about 400 million, from about 200 million to about 400 million, from about 250 million to about 400 million, from about 300 million to about 400 million, from about 350 million to about 400 million, from about 50 million to about 350 million, from about 100 million to about 300 million, from about 150 million to about 250 million, from about 50 million to about 100 million, from about 100 million to about 150 million, from about 150 million to about 200 million, from about 250 million to about 300 million, or from about 300 million to about 350 million anti-BAFF-R CAR⁺ cells). In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer (e.g., one or more B-cell cancers) to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CART cells) provided herein) can be administered to a mammal (e.g. a human) to reduce the number of cancer cells within the mammal and/or to increase the survival of the mammal suffering from cancer.

In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) having cancer (e.g., a BAFF-R⁺ cancer) to reduce or eliminate one or more symptoms of the cancer. Examples of symptoms of a cancer (e.g., a B-cell cancer) that can be reduced using a composition comprising one or more binders described herein include, without limitation, bleeding gums, bone pain, fever, frequent infections, frequent nosebleeds, severe nosebleeds, lumps (e.g., lumps caused by swollen lymph nodes in and around the neck, armpits, abdomen, and/or groin), pale skin, shortness of breath, weakness, fatigue, and decrease in energy.

Any appropriate mammal having cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). Examples of mammals that can be treated as described herein include, without limitation, humans, non-human primates (e.g., monkeys), dogs, cats, horses, cows, pigs, sheep, mice, and rats. For example, a human having a cancer can be treated with a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein).

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. A cancer that can be treated as described herein can be a primary cancer or a metastatic cancer. In some cases, a cancer that can be treated as described herein can be a refractory or relapsed cancer. For example, a cancer that can be treated as described herein can be an antigen-negative (e.g., CD19-negative) relapsed cancer. In some cases, a cancer that can be treated as described herein can include one or more solid tumors. In some cases, a cancer that can be treated as described herein can be a blood cancer. For example, a cancer that can be treated as described herein can be a B-cell cancer (e.g., a cancer resulting from malignant transformation of a B-cells or a B-cell precursor). For example, a cancer than can be treated as described herein can be a plasma cell cancer (e.g., a cancer resulting from malignant transformation of a plasma cell). Examples of cancers that can be treated as described herein include, without limitation, CLLs, acute lymphoblastic leukemias (ALLs), hairy cell leukemias, follicular lymphomas, non-Hodgkin's lymphomas (NHLs), Hodgkin's lymphomas, multiple myelomas, Waldenstrom's macroglobulinemias, diffuse large B-cell lymphomas (DLBCLs), and intravascular large B-cell lymphomas. In some cases, a mammal (e.g., a human) having a BAFF-R⁺ cancer (e.g., a BAFF-R⁺ B-cell cancer) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer (e.g., one or more B-cell cancers) without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CART cells) provided herein) (e.g., a pharmaceutical composition provided herein) can be an amount that increases the survival time of a mammal having cancer (e.g., one or more B-cell cancers) as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). In another example, an effective amount of cells (e.g., T cells) expressing a binder (e.g., CAR) provided herein can be from about 25 million anti-BAFF-R CAR⁺ cells to about 400 million anti-BAFF-R CART cells (e.g., from about 25 million to about 350 million, from about 25 million to about 300 million, from about 25 million to about 250 million, from about 25 million to about 200 million, from about 25 million to about 150 million, from about 25 million to about 100 million, from about 25 million to about 50 million, from about 50 million to about 400 million, from about 100 million to about 400 million, from about 150 million to about 400 million, from about 200 million to about 400 million, from about 250 million to about 400 million, from about 300 million to about 400 million, from about 350 million to about 400 million, from about 50 million to about 350 million, from about 100 million to about 300 million, from about 150 million to about 250 million, from about 50 million to about 100 million, from about 100 million to about 150 million, from about 150 million to about 200 million, from about 250 million to about 300 million, or from about 300 million to about 350 million anti-BAFF-R CAR⁺ cells). The effective amount can remain constant or can be adjusted as a sliding scale or variable dose depending on the mammal's response to treatment. Various factors can influence the actual effective amount used for a particular application. For example, the severity of cancer when treating a mammal having cancer (e.g., one or more B-cell cancers), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer (e.g., one or more B-cell cancers) without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer (e.g., one or more B-cell cancers) as compared to a control mammal having comparable cancer and not treated with the composition. For example, an effective frequency of administration of a pharmaceutical composition provided herein such as a pharmaceutical composition containing one or more binders provided herein can be from about twice daily to about once a week (e.g., about once daily). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer (e.g., one or more B-cell cancers), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer (e.g., one or more B-cell cancers) as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer (e.g., one or more B-cell cancers), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a BAFF-R polypeptide (e.g., a human BAFF-R polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, peptide, colorimetric, or fluorescent label). The labelled binder can be used to detect the presence or absence of a BAFF-R polypeptide (e.g., a human BAFF-R polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a BAFF-R polypeptide (e.g., a human BAFF-R polypeptide). In some cases, a binder (e.g., an antibody, antigen binding fragment, and/or antibody domain) provided herein can be immobilized, e.g., on a support, and retention of a BAFF-R polypeptide (e.g., a human BAFF-R polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a BAFF-R polypeptide (e.g., a human BAFF-R polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be used (e.g., in an adoptive T cell therapy such as a CAR-T cell therapy) to treat a mammal having a disease or disorder other than cancer. For example, one or more T cells (e.g., CAR-T cells) expressing (e.g., engineered to express) a CAR described herein (e.g., a CAR having the ability to bind to a BAFF-R polypeptide). can be used to treat one or more autoimmune indications. Examples of autoimmune indications that can be treated by administering one or more T cells (e.g., CAR-T cells) expressing (e.g., engineered to express) a CAR described herein (e.g., a CAR having the ability to bind to a BAFF-R polypeptide) include, without limitation, organ transplant rejection, autoimmune diseases (e.g., systemic lupus erythematosus and rheumatoid arthritis), HLA sensitization, vasculitis, and multiple sclerosis.

In cases where composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) is used to treat organ transplant rejection and/or to treat HLA sensitization, the composition containing one or more binders provided herein can be administered to a mammal having a transplanted organ or to a mammal preparing to have an organ transplant (e.g., an allogeneic transplant or an autologous transplant). For example, a composition containing one or more binders provided herein can be administered to a mammal having a transplanted organ to reduce or eliminate one or more symptoms of organ transplant rejection in that mammal. Examples of symptoms of organ transplant rejection include, without limitation, impaired organ function, pain and swelling in the area around the transplanted organ, flu-like symptoms, and unexplained changes in blood pressure. For example, a composition containing one or more binders provided herein can be administered to a mammal preparing to have an organ transplant to delay or prevent organ transplant rejection in that mammal.

A composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be used to treat a mammal (e.g., a human) having any type of organ transplant. For example, a composition containing one or more binders provided herein can be used to delay or prevent organ transplant rejection of any type of organ transplant. Examples of organs that can be transplanted and where the transplanted organ can be treated as described herein (e.g., to delay or prevent organ transplant rejection) include, without limitation, kidneys, lungs, livers, hearts, pancreas, and bone marrow.

The invention will be further described in the following examples, which do not limit the scope of the invention described in the claims.

### EXAMPLES

### Example 1: CAR T-Cell Therapy Targeting BAFF-R in B-Cell Lymphoid Malignancies

This Example describes the design and characterization of molecules that can bind to a BAFF-R polypeptide.

### Materials and Methods

### Cell Lines

The cell lines of Nalm-6, MEC-1, and Z138 were purchased from Deutsche Sammlung von Mikroorganismen und Zellkulturen GmbH (Braunschweig, Germany); Jurkat and 293FT cell lines were obtained from Thermo Fisher Scientific (Waltham, Massachusetts, USA) and American Type Culture Collection (Manassas, Virginia, USA), respectively. Cells were maintained in either 90% RPMI 1640, Iscove's MDM, or 90% Dulbecco's Modified Eagle Medium (Thermo Fisher) supplemented with 10% heat-inactivated fetal bovine serum (FBS; Thermo Fisher). Cell lines were authenticated by flow cytometry. A BAFF-R knock out in Nalm-6 (BAFF-R KO Nalm-6) and a CD19 knock out in Nalm-6 (CD19 KO Nalm-6) were generated, and luciferase expressing human cell lines were generated for in vivo experiments.

PBMCs from healthy volunteer donors were isolated via leukapheresis using Leukocyte Reduction System (LRS) cones. For the generation of CAR-T cells, naïve and memory T cell (Tn/mem) populations were isolated from PBMCs in a three-step procedure based upon the absence of CD14/CD25 and expression of CD62L on these cells. Negative selection of both CD14 and CD25 and positive selection of CD62L were performed using CD14, CD25, and CD62L microbeads per the manufacturer (Miltenyi Biotech, Germany) protocol.

For experiments in which the target cell population was B-cells, fifty milliliters of blood were obtained from adult subjects with a diagnosis of CLL at the time of a clinically indicated blood draw. Patient and disease characteristics of these subjects were recorded. Tumor cells in each sample ranged from 80 to 98% for leukapheresis or blood. The minor population of T cells present in these tumor sample was removed to avoid confounding results for the BAFF-R CAR-T cell testing. The B-cells were isolated using the EasySep™ Direct Human B-cell Isolation Kit (Stemcell Technologies, Vancouver, Canada), per the manufacturer protocol. PBMCs and enriched B-cells were incubated with anti-BAFFR-AF647/anti-CD3-BV605 antibody for 30 minutes at 4° C. Cells were then washed as before and resuspended in 400 μL of FACS for analysis on an Attune flow cytometer (Thermo Fisher). FMO was set as a negative control.

### BAFF-R Antibody Development

A monoclonal antibody targeting human BAFF-R was generated. To enable overexpressing BAFF-R, the cDNA encoding human BAFF-R (Origene, Rockville, MD, USA) was cloned into a lentivirus gene delivery system (pCDH cDNA Cloning and Expression Lentivectors, System Biosciences, Palo Alto, CA.). NIH/3T3 or 293FT cells were infected with the pCDH-BAFF-R lentivirus, followed by selection of 1 μg/mL puromycin (Sigma-Aldrich, St. Louis, MO, USA) for 1 week. Single-cell clones were established from sorted BAFF-R positive NIH/3T3 or BAFF-R positive 293FT cells, and a BAFF-R expressing NIH/3T3 cell clone were used as the immunogen for BAFF-R monoclonal antibody development. The BAFF-R positive 293FT cells were used for screening monoclonal antibody clones.

Several crude supernatants were screened using a 5×5-fold serial dilutions from hybridoma clones were incubated with BAFF-R-293FT cells for 30 minutes at 4° C. Following two washes in FACS (PBS with 2% bovine serum albumin), cells were incubated with R-PE conjugated goat anti-mouse IgG for 30 minutes at 4° C. Cells were then washed as before and resuspended in 400 μL FACS for analysis on an Attune flow cytometer (Thermo Fisher). Mean fluorescence intensity values in the R-PE channel were collected. Undiluted supernatants were incubated with 293FT cells as a negative control. Representative data are included to show the process.

The lead clone was identified and advanced to purification. Purified monoclonal antibody C21 was incubated with BAFF-R-293FT cells as well as 293FT cells for 30 minutes at 4° C. Following two washes in FACS (PBS with 2% bovine serum albumin), cells were incubated with R-PE conjugated goat anti-mouse IgG for 30 minutes at 4° C. Cells were then washed as before and resuspended in 400 μL FACS for analysis on an Attune flow cytometer (Thermo Fisher). BAFF-R-293FT cells were incubated with R-PE conjugated anti-mouse IgG as a negative control. Anti-BAFF-R monoclonal antibody development was monitored by R-phycoerythrin conjugated goat anti-mouse IgG (BD Bioscience, San Jose, California).

### Antibodies and Flow Cytometry

Productive lentiviral transduction was identified with surface expression of truncated epidermal growth factor receptor (tEGFR) by incubating T cells for 30 minutes at 4° C. with anti-EGFR-BV421 (clone AY13; BioLegend, San Diego, California, USA), and T cell identity was confirmed similarly using anti-CD3-BV605 (clone SK7, BD Biosciences). The functional activity of live T cells (Sytox Blue, Thermo Fisher or propidium iodide, Thermo Fisher) was imaged using antibodies and flow cytometry. Non-transduced T cells were washed with buffer and incubated for 30 minutes at 4° C. with anti-CD107a APC (clone H4A3; BD Biosciences, San Jose, California, USA), anti-CD4 PE (phycoerythrin clone SK1, BD Biosciences), and anti-CD8 APC-Cy7 (clone SK1, BD Biosciences). Gating was on either CD4 or CD8. To assess antigen induced activation and degranulation of CAR-T cells, T cells were incubated for 30 minutes at 4° C. with anti-CD107a APC, anti-CD4 PE, anti-CD8 APC-Cy7, anti-EGFR BV421, and antigen specific antibodies (anti-CD19 APC (clone HIB19; BD Biosciences) or anti-BAFF-R Alexa Fluor 647 (clone 11C1; BD Biosciences)). Activated CAR-T cells in the degranulation assays were CD4⁺ EGFR⁺ CD107a⁺ or CD8⁺ EGFR⁺ CD107a⁺. Data were acquired on a BD Fortessa or on MACSQuant Analyzer 10 (Miltenyi Biotech) and analyzed using FlowJo™ Version 10 software.

### CAR-T Cell Production

A second-generation BAFF-R-CAR was generated consisting of a BAFF-R antibody scFv (described herein), CD28 transmembrane domain, CD28 and CD3ζ intracellular signaling domains, and a tEGFR included as a marker and suicide switch (described herein). The CAR cDNA was cloned into pHIV.7 lentiviral vector. CD19-CAR was generated in the same way, replacing BAFF-R antibody scFv with CD19 antibody scFv (Qin et al., Science Translational Medicine, 11(511) 2019). Lentiviruses were produced in 293FT cells, concentrated, and titered with Jurkat cells. Tn/mem were isolated from healthy donor PBMCs and divided into two aliquots. One aliquot advanced to be activated and expanded as non-transduced (non-CAR) T cells whereas the remaining cells advanced to CAR-T cell production. These T cells were activated with human T-Activator CD3/CD28 beads (Life Technologies) for 24 hours followed by transduction with lentivirus encoding CAR at multiplicity of infection (MOI)=1. The CAR-T cells were further activated with CD3/CD28 bead stimulation for seven days after which the beads were removed, and CAR-T cells were permitted to expand for an additional seven days. Non-transduced T cells from the same donor were also expanded following the CAR-T cell protocol, and were used as a control. CAR-T cell proliferation was monitored by determining viable cell numbers using trypan blue exclusion and the Bio-Rad TC20™ Automated Cell Counter (Bio-Rad Laboratories, Inc. California, USA). Each batch of CAR-T cells destined for research testing was evaluated for cell quality with Fold Expansion (>25) and Viability (>70%, as determined by Trypan Blue staining) and CAR-T cell specific characterization with Identity (>70%, as determined by flow cytometry for CD3 positive cells) and Potency (>10%, as determined by flow cytometry for EGFR positive T cells). The clinical grade batches had additional safety testing from adventitious viral agents that includes determining lentiviral copy number (<5 copies of Woodchuck Hepatitis Virus Posttranscriptional Regulatory Element (WPRE) real-time qPCR method and <2.5 copies/50 ng DNA of VSVG (encodes envelope gene sequences) detected in a qPCR assay).

### In Vitro Functional Assays

To determine functional potency, CAR-T cells were incubated with target cells at an E:T ratio (specified in figure legend) for 6 hours with CD107a-APC antibody, as a marker of degranulation. CD107a (also referred to as lysosomal-associated membrane protein-1 (LAMP-1)) is a protein that lines the interior of secretory, cytolytic granules that becomes surface accessible when T cell granules migrate to the plasma membrane in response to an immune responsive signal. CAR-T cells and target cells were coincubated for 72 hours at an E:T ratio of 4:1, unless specifically mentioned. The supernatant was collected after 72 hours and granzyme B was measured using an ELISA (U-PLEX Human Granzyme B Assay ELISA kit, Meso Scale Diagnostics, Rockville, MD, USA).

### In Vivo Modeling

NOD scid gamma (NSG) mouse breeding pairs were purchased from The Jackson Laboratory (stock no. 005557) to establish a NSG breeding colony. Mice were housed in a pathogen-free animal facility. NSG mice (8-12 weeks old) received an intravenous (IV) challenge with a luciferase-expressing tumor cell line, were randomized into test groups, and were treated with a single IV dose of CAR-T cells. Numbers of animals per test group are listed in the figure legends. The tumor burden was quantified by bioluminescent signal intensity on isoflurane anesthetized mice that received a subcutaneous injection of D-luciferin (150 μg luciferin/1 g mouse body weight) ten minutes prior to imaging with IVIS imaging (Perkin Elmer, Waltham, MA). Body weight was monitored, and survival data were reported in Kaplan-Meier plots. Animals with an excessive tumor burden were humanely euthanized. In some cases, although there was no or limited tumor burden, animals presented with weight loss, lethargy, and/or poor appearance and required humane euthanasia. Cursory necropsy suggested graft-versus-host disease. Experiments with mice were performed with either all male mice or all female mice. Experiments with the cell lines were replicated at least twice.

### Statistical Analysis

Statistical analyses (means with associated standard deviations) were performed using Prism Version software (GraphPad, San Diego, USA). Typical comparisons were between non-CAR and antigen-specific CAR with the following convention: * for P<0.05; ** for P<0.01; *** for P<0.001. Unpaired t-test comparisons were performed with granzyme B release data and a log rank test was performed for the animal studies. A bar over a data set pair was also used to indicate statistical comparison.

### Results

### Generation of an Anti-BAFF-R MC10029 CAR

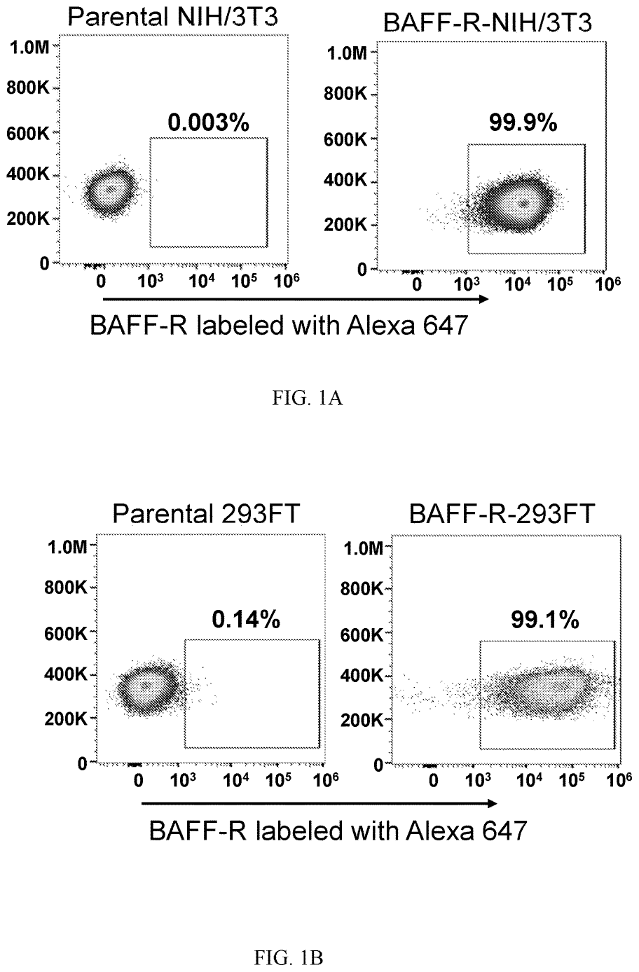

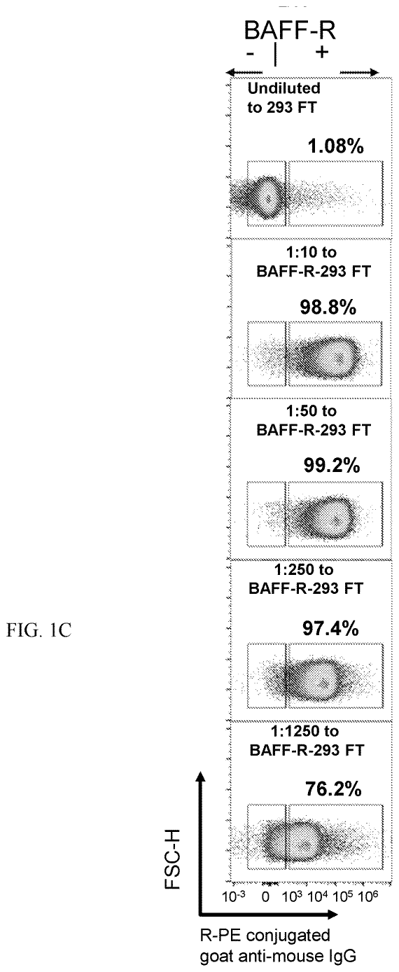

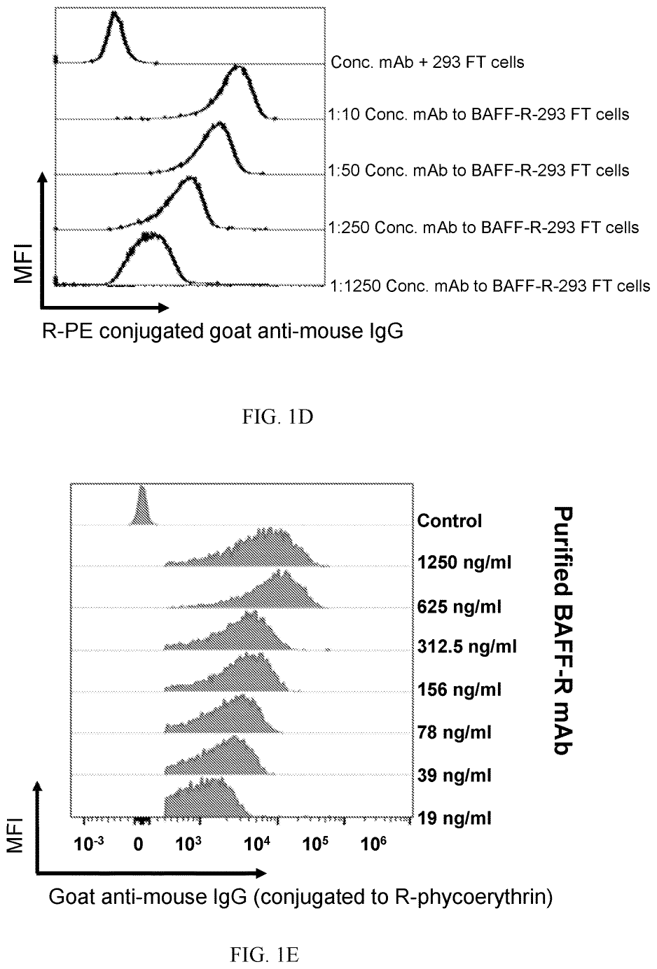

A BAFF-R expressing NIH/3T3 cell line was generated and used to immunize mice (FIG. 1A). After successful immunization and hybridoma development, candidate hybridoma clone supernatants were provided for screening against BAFF-R expressing 293FT cells (FIG. 1B). Putative clones were screened by monitoring the serial dilution of raw supernatant with increasing concentrations of BAFF-R expressing 293FT cells, while monitoring mouse IgG by flow cytometry (FIGS. 1C and 1D). The lead BAFF-R monoclonal antibody was then purified (FIG. 1E). The lead clone, C21, was sequenced, and the sequence of the heavy chain variable region and the light chain variable region was identified for advancement into CAR design.

A second generation BAFF-R CAR was generated using a clinical vector (Dong et al., Cancer Immunology, Immunotherapy, 69(10), 2139-2145, 2020). The BAFF-R CAR construct included the single chain variable fragment of the monoclonal antibody to BAFF-R with the following elements in tandem: hinge, CD28 transmembrane domain, CD28 signaling domain, CD3ζ signaling domain, and tEGFR (FIG. 2A). Research grade BAFF-R CAR-T cells were generated with quality control measurements of cell quality, CAR-T cell specific characterization, and CAR-T cell proliferation (FIGS. 2B, 2C, and 2D). This generation of CAR T cells allowed for production comparisons and ensured the quality of CAR-T cells for experimental evaluation, specifically in the in vivo experiments.

### MC10029 CAR-T Cells Show Antigen-Specific Cytotoxicity of Against ALL.

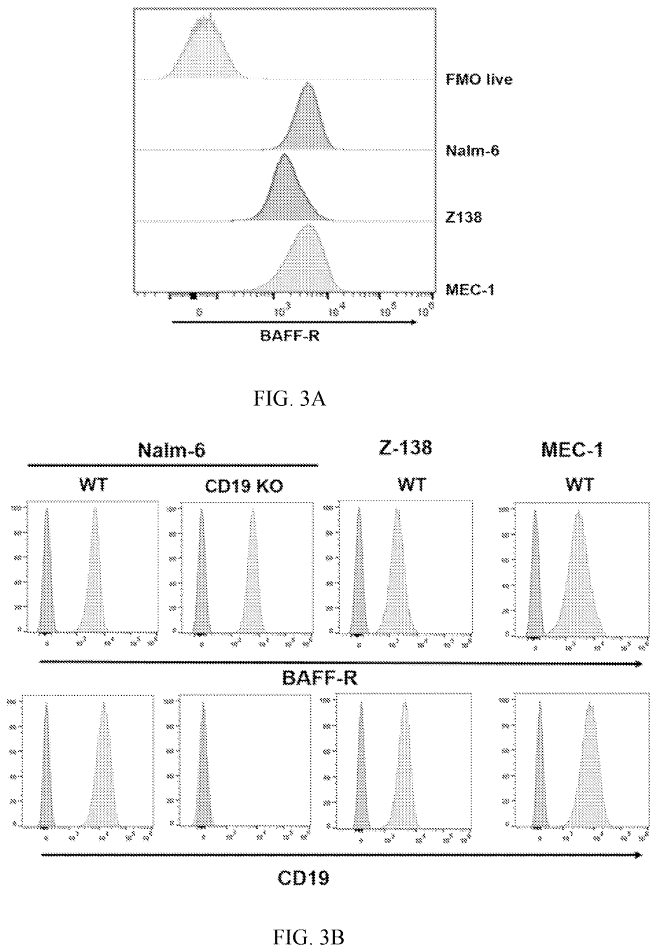

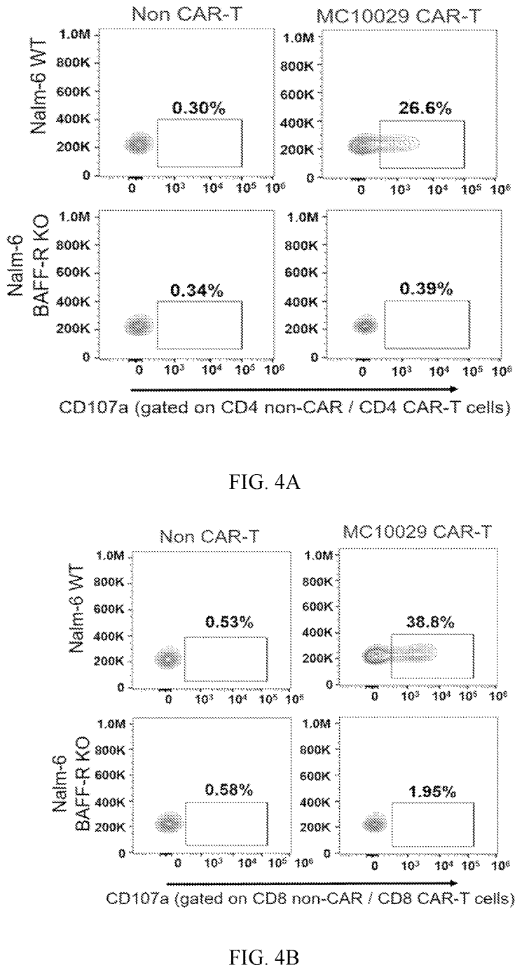

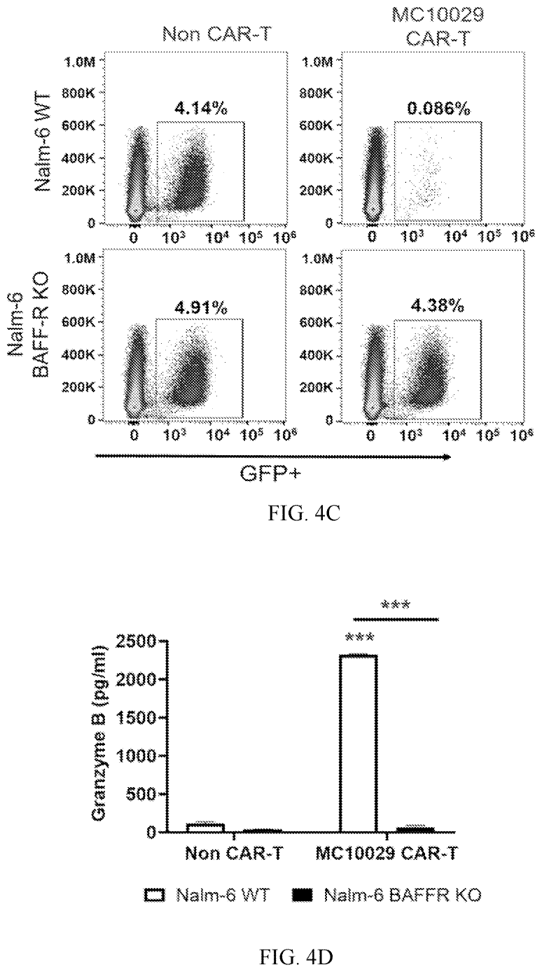

The expression of BAFF-R in the cell lines used to characterize MC10029 CAR T cell function (FIG. 3A) was confirmed. Nalm-6 expressed both BAFF-R and CD19 (FIG. 3B). The antigen-specific cytotoxic properties of BAFF-R CAR T cells were shown after incubation with either wild-type Nalm-6 or BAFF-R KO Nalm-6 cell lines. The antigen specific cytotoxicity was measured by a CD107a degranulation assay and either gated on CD4 or CD8 to highlight the activity of these specific MC10029 CAR T cell populations (FIG. 4A and FIG. 4B, respectively). Granzyme B released into the supernatant was observed when MC10029 CAR T cells were incubated with wild-type Nalm-6 but not observed in non-transduced T cell controls or with BAFF-R KO Nalm-6 cells (FIG. 4C). Antigen-specific cytotoxicity of MC10029 CAR T cells was further examined by incubating the CAR T cells with GFP-labeled target cells. Cytolysis, as determined with the loss of GFP labeled target cells, was observed only when MC10029 CAR-T cells were incubated with the GFP-labeled WT Nalm-6 (FIG. 4D).

### MC10029 CAR-T Cells Show In Vivo Antitumor Effects Targeting ALL

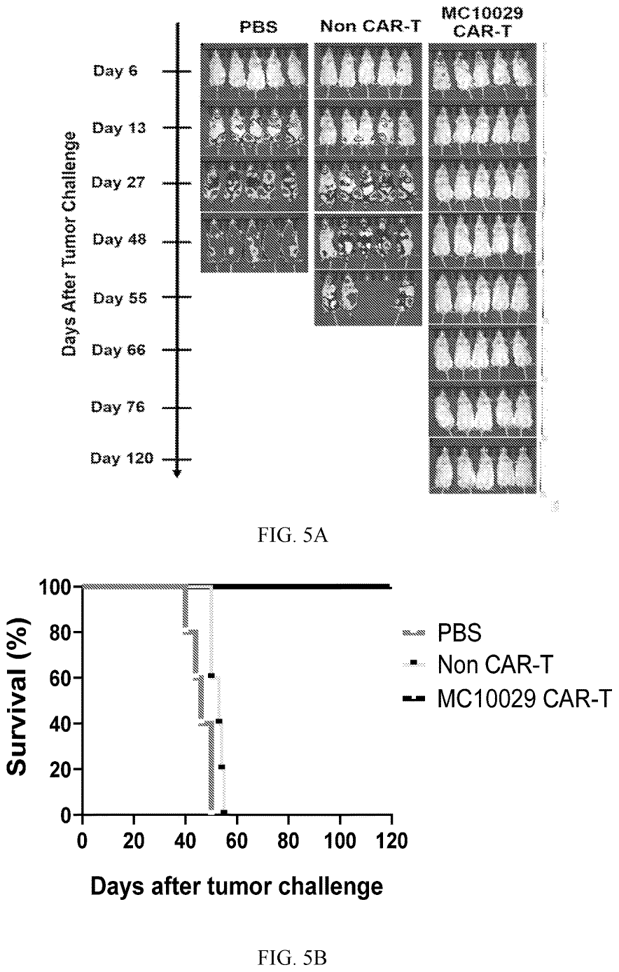

The therapeutic efficacy of BAFF-R CAR T cells was tested in an NSG mouse model in which the mice were challenged with luciferase labeled-human Nalm-6 tumor cells (0.25×10⁶ cells). Mice were randomly assigned into groups of five mice for a single infusion of either vehicle (PBS), non-transduced T cells (10×10⁶ cells), or BAFF-R CAR T cells (2×10⁶ cells) that were generated from the same donor. Bioluminescence imaging of the NSG mice after IV tumor challenge and after CAR T cells treatment was used to monitor tumor response to treatment along a timeline (FIG. 5A). Tumor presence and long-term survival following these three treatments was evaluated (FIG. 5B). A Kaplan-Meier plot shows these data as percentage of survival versus time after tumor challenge. Tumor growth and death were noted in the PBS treated mice within 42 days; the non-transduced T cell treated group also showed advanced tumor progression and subsequent death. The MC10029 CAR T cell treated mice showed a marked disappearance of the Nalm-6 tumor cells, and a statistically significant survival rate to 120 days when the study was terminated.

### MC10029 CAR-T Cells Remained Effective in Models of CD19 Antigen Loss

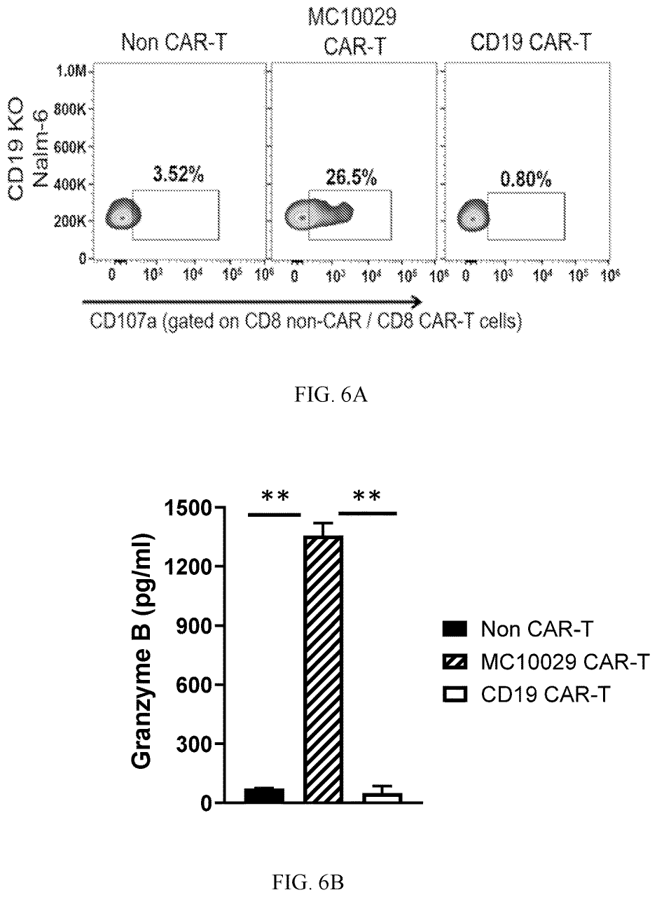

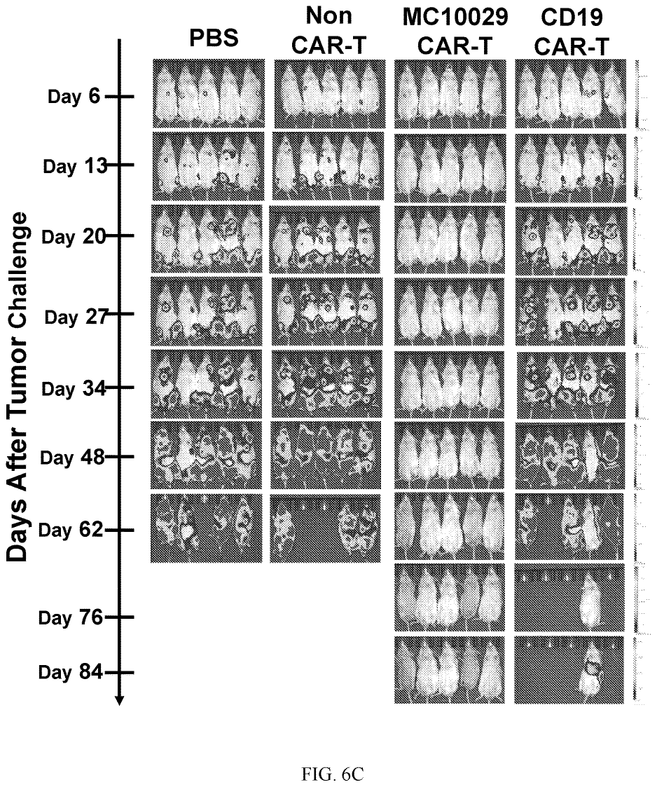

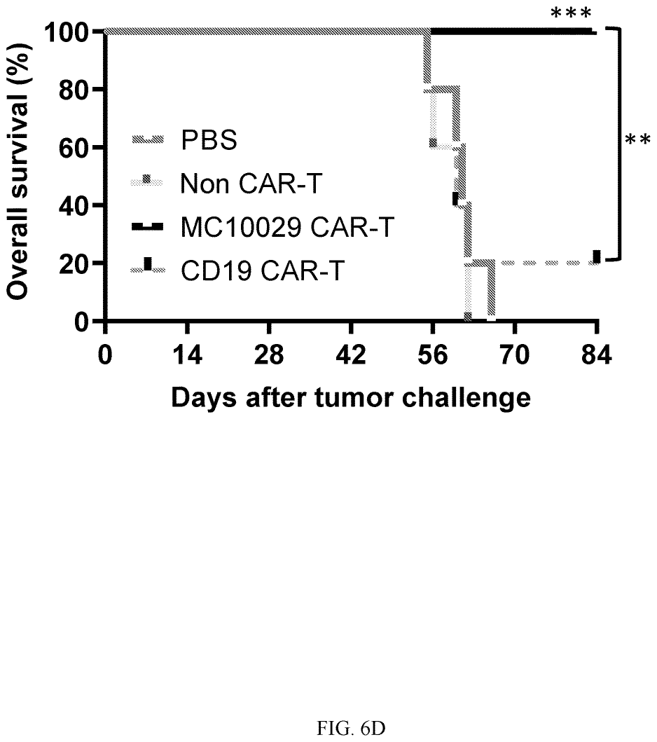

A Nalm-6-based model was generated that was engineered to be deficient in CD19 to mimic relapse/refractory disease. It was shown that MC10029 CAR-T cells retain antigen specific cytotoxic function, specifically CD8 CAR-T cells, targeting the CD19 KO Nalm-6 cell line while non-transduced T cells and CD19 CAR-T cells showed only background activity targeting the CD19 KO Nalm-6 cell line (FIG. 6A). This activity profile was confirmed by measuring granzyme B, showing a robust activity of MC10029 CAR T cells compared to non-transduced T cells and CD19 CAR T cells (FIG. 6B). The efficacy of CAR T cells was tested in an in vivo relapse model in which NSG mice were injected with luciferase labeled-human CD19 deficient Nalm-6 tumor cells (0.25×10⁶ cells). Seven days after tumor challenge, mice (5 mice per treatment group) received one of four treatments: PBS (vehicle), non-transduced T cell from same donor (10×10⁶ cells), MC10029 CAR T cells (2×10⁶ cells), or CD19 CAR T cells (2×10⁶ cells). Bioluminescence imaging of the NSG mice after intravenous tumor challenge and after treatment with either CAR T cells, non-transduced T cells, or PBS was used to follow tumor changes along the provided timeline (FIG. 6C). The control groups of PBS or non-transduced T cells were euthanized at or around 60 days due to excessive tumor burden. 80% of the mice in the CD19 CAR T cell treatment group showed similar tumor burden as the controls with one mouse surviving to the termination of the experiment after 84 days. The MC10029 CAR T cell treatment group showed tumor eradication and survival of all mice for the 84 days of the experiment. A Kaplan-Meier plot shows these data as percentage of survival versus time post tumor challenge (FIG. 6D). Collectively, these results suggest BAFF-R CAR T cells would be effective in cases of CD19 antigen escape.

### MC10029 CAR-T Cells Show Antigen-Specific Cytotoxicity of Against Lymphoma

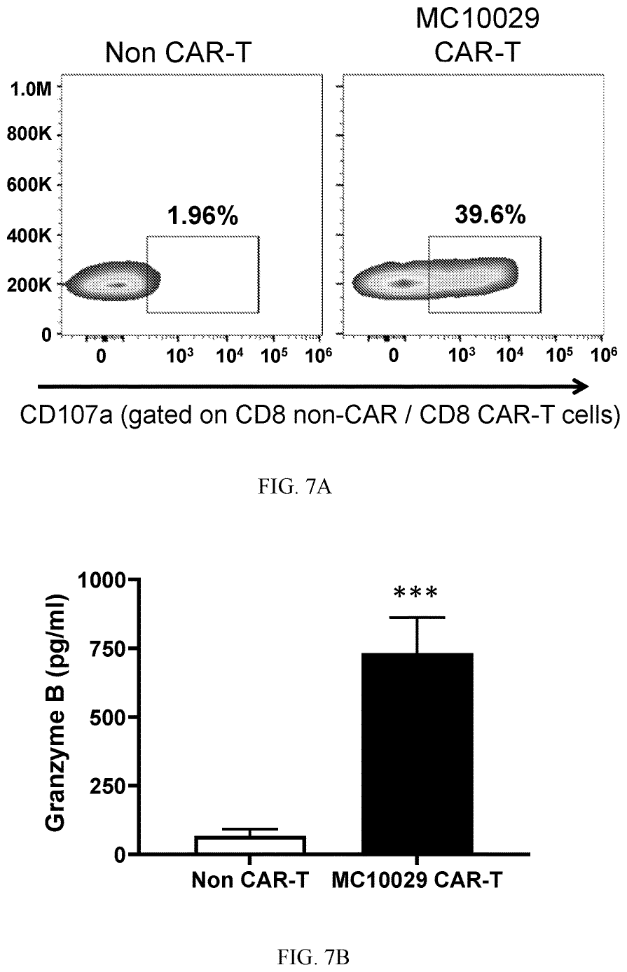

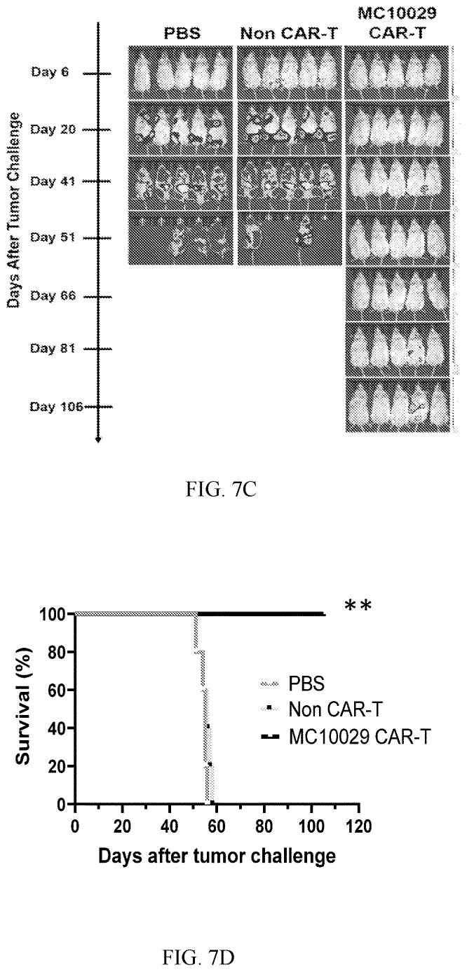

Z138, a non-Hodgkin lymphoma (NHL) cell line, expressed both BAFF-R and CD19 (FIG. 3A), was used to test the cytotoxic potential of MC10029 CAR T cells against a lymphoma cell line. Antigen-specific cytotoxicity of MC10029 CAR T cells against Z138 was confirmed using a CD107a degranulation assay that was gated on CD8 (FIG. 7A). Granzyme released into the supernatant was observed when MC10029 CAR T cells were incubated with Z138 but not observed in non-transduced T cells controls (FIG. 7B). Efficacy of MC10029 CAR T cells was tested in an in vivo model in which NSG mice were injected with luciferase labeled-human Z138 tumor cells (0.5×10⁶ cells). Allowing seven days after tumor challenge for tumor engraftment, mice received one of three treatments: PBS (vehicle), non-transduced T cell from same donor) (10×10⁶ cells), or MC10029 CAR T cells (2×10⁶ cells). Bioluminescence imaging of the NSG mice after intravenous tumor challenge and after CAR T cells treatment was used to monitor tumor response to treatment along the provided timeline (FIG. 7C). The controls groups of PBS or non-transduced T cells were euthanized within 56 days due to excessive tumor burden. The MC10029 CAR T cell treatment group showed tumor eradication and survival of all mice for the 106 days of the experiment. A Kaplan-Meier plot shows these data as percentage of survival versus time post tumor challenge (FIG. 7D). MC10029 CAR T cells recognized and killed Z138 tumor cells when presented in in vitro and in vivo models.

### MC10029 CAR-T Cells Show Antigen-Specific Cytotoxicity of Against CLL

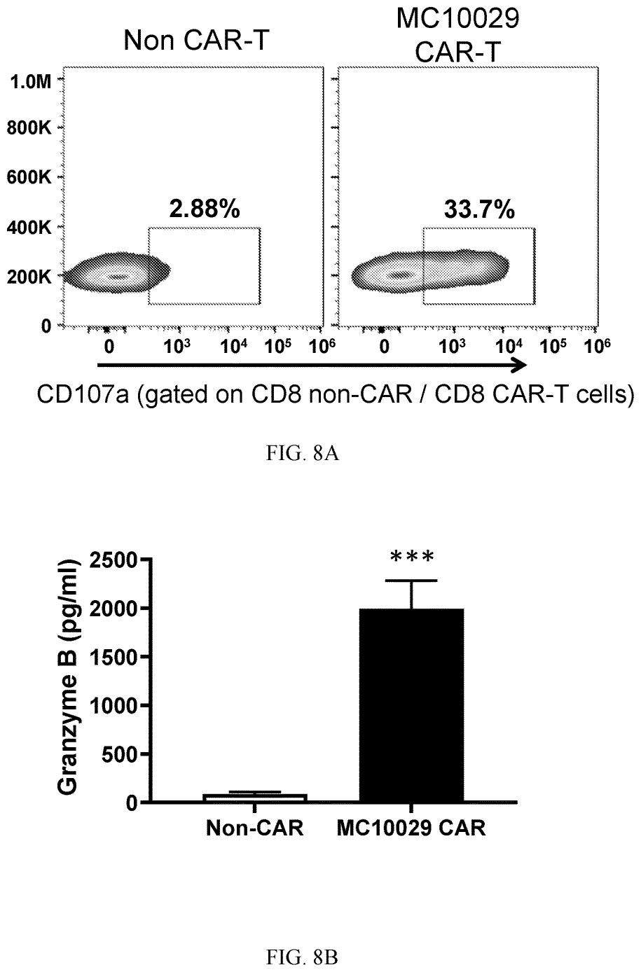

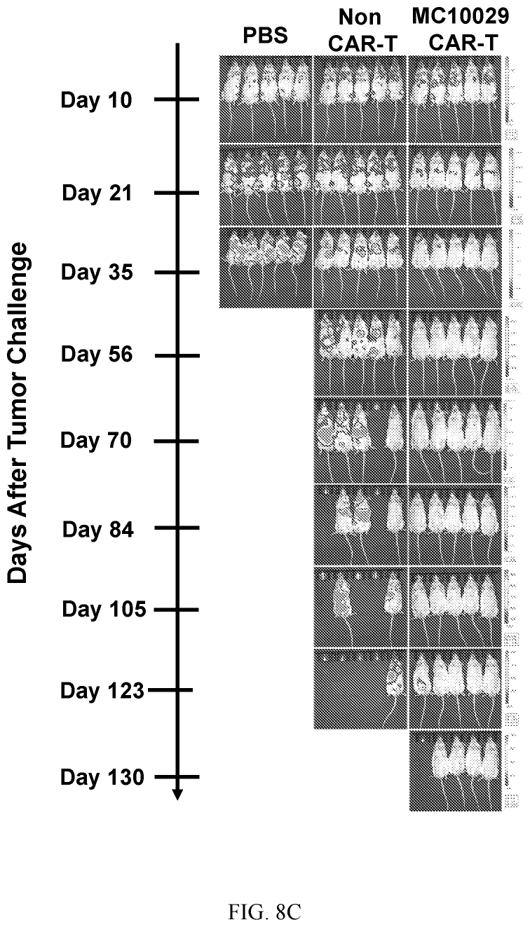

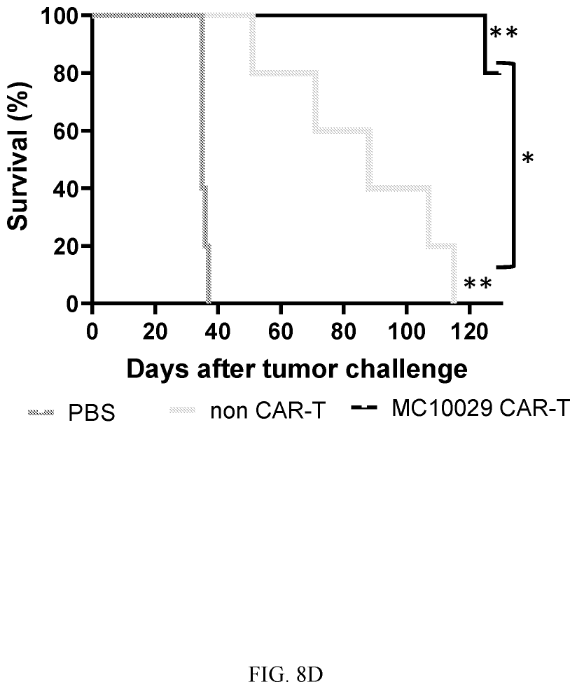

MEC-1, a CLL cell line, expressed both BAFF-R and CD19 (FIG. 3A). The effect of incubating MC10029 CAR T cells with MEC-1 was examined in both in vitro and in vivo models. Antigen-specific cytotoxicity of MC10029 CAR T cells against MEC-1 was confirmed using a CD107a degranulation assay that was gated on CD8 (FIG. 8A). Granzyme B released into the supernatant was observed when MC10029 CAR T cells were incubated with MEC-1 but not observed in non-transduced T cell controls (FIG. 8B). Efficacy of MC10029 CAR T cells was evaluated in an in vivo model in which NSG mice were injected with luciferase labeled-human MEC-1 tumor cells (1.0×10⁶ cells). Allowing ten days after tumor challenge for tumor engraftment, mice received one of three treatments: PBS (vehicle), non-transduced T cell from same donor (10×10⁶ cells), or MC10029 CAR T cells (2×10⁶ cells). Bioluminescence imaging of the NSG mice after intravenous tumor challenge and after treatment was used to monitor tumor progression along the provided timeline (FIG. 8C), and survival was plotted (FIG. 8D). The PBS group was euthanized within 37 days due to excessive tumor burden; the non-transduced T cell treated group showed steady tumor progression over the length of the experiment with gradual loss of mice due to tumor burden. The MC10029 CAR T cell treatment group showed tumor regression within one month and survival of 80% of mice for the 130 days of the experiment. MC10029 CAR T cells recognized and killed MEC-1 tumor cells when presented in vitro and in vivo models, highlighting its potential to treat CLL.

### MC10029 CAR-T Cells Targeted Primary Human CLL Cells

MC10029 CAR-T cells were tested against primary B cells isolated from subjects with CLL. The six selected subjects were three males and three females with an age range of 56 to 83 (See Table 1).

TABLE 1

| Demographic Data | | | |
|---|---|---|---|
| Identifier | Disease | Gender | Age (Years) |
| 1 | B-CLL | Female | 77 |
| 2 | B-CLL | Male | 63 |
| 3 | B-CLL | Female | 83 |
| 4 | B-CLL | Male | 78 |
| 5 | B-CLL | Female | 65 |
| 6 | B-CLL | Male | 56 |

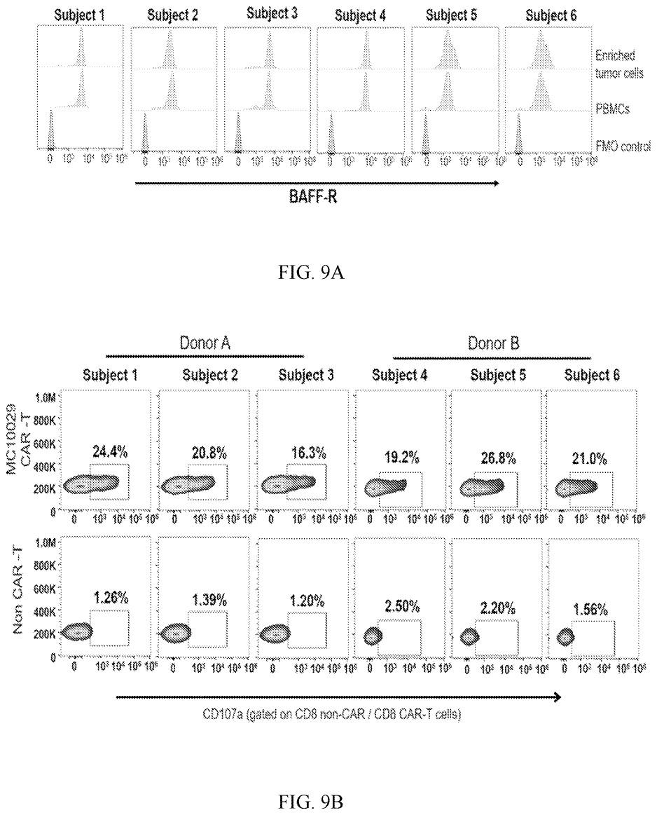

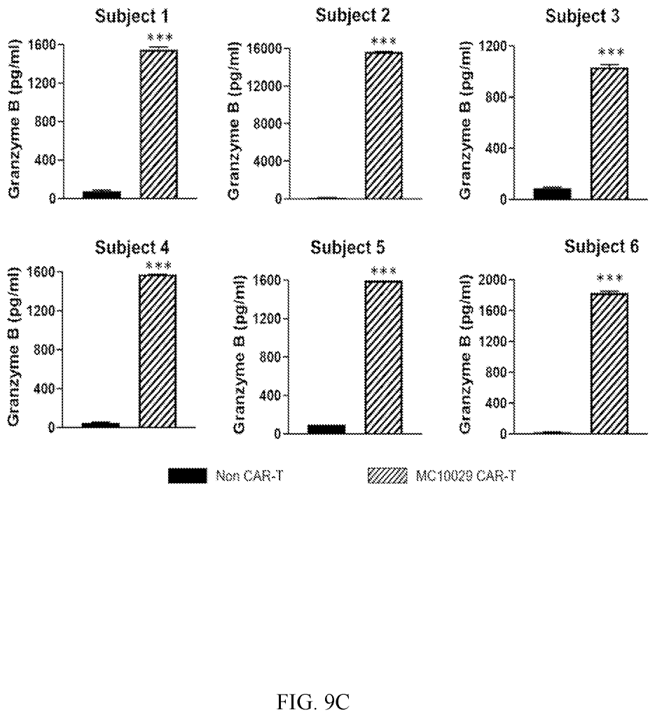

B cells were enriched from PBMCs of each subject, and BAFF-R expression was confirmed (FIG. 9A). Two batches of MC10029 CAR T cells and non-transduced T cells that were generated from two healthy donor were generated and were then incubated with the six human primary CLL tumor cells. By gating on EGFR and CD8, the percentage of CD8⁺ MC10029 CAR T cells (FIG. 9B, top panels) that were activated after incubation with the isolated primary CLL tumor cells were identified. The percentages of activated MC10029 CAR T cells were comparable for B-cells isolated from all subjects. Non-transduced T cells generated from the two healthy donors showed background activities against the B-cells that were also comparable for the collection of CLL tumor cells (FIG. 9B, lower panels). Complementary cytotoxic activity was determined by measuring the release of granzyme B into the medium of MC10029 CAR T cell or non-transduced T cells that were incubated with CLL tumor cells for 72 hours. Incubation with CLL tumor cells resulted in the statistically significant release of granzyme B from MC10029 CAR T cells compared to non-transduced T cells (FIG. 9C).

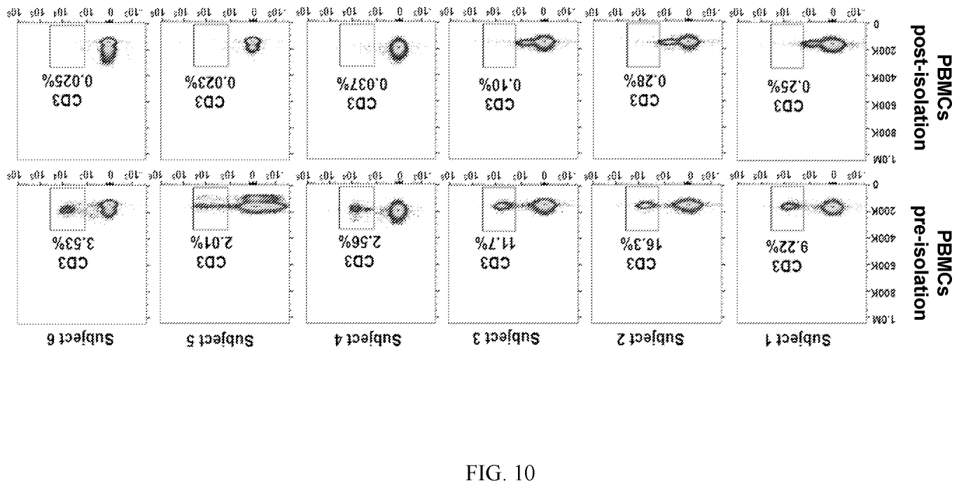

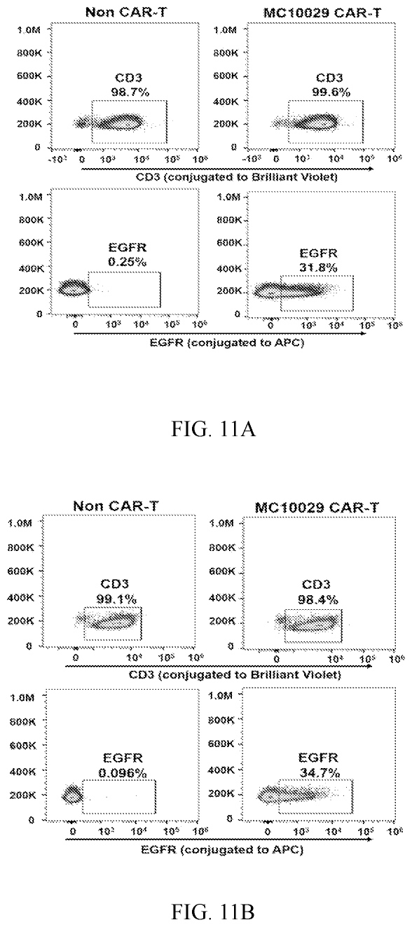

Controls were performed to ensure the quality of both the B cells that were used and the CAR-T cells that were generated. In order to confirm that MC10029 CAR T cell activity was targeting primary CLL tumor cells, the PBMCs were enriched for tumor cells, CLL B-cells. Using flow cytometry, the population of CD3 positive cells from six subjects with CLL were characterized in the original PBMC samples (FIG. 10, top panels) and after B-cells enrichment (FIG. 10, bottom panels) to confirm endogenous T cell removal.

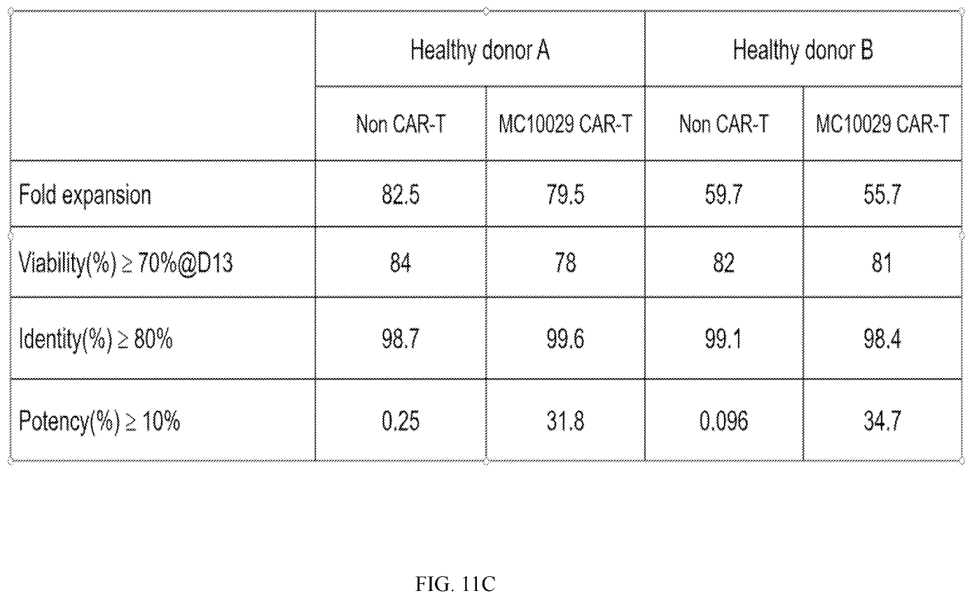

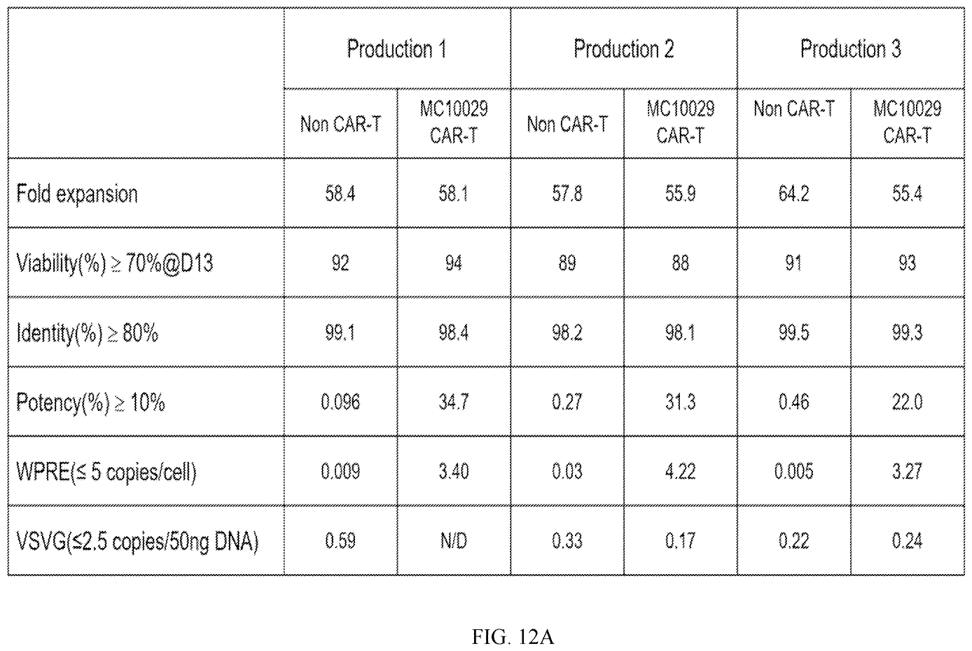

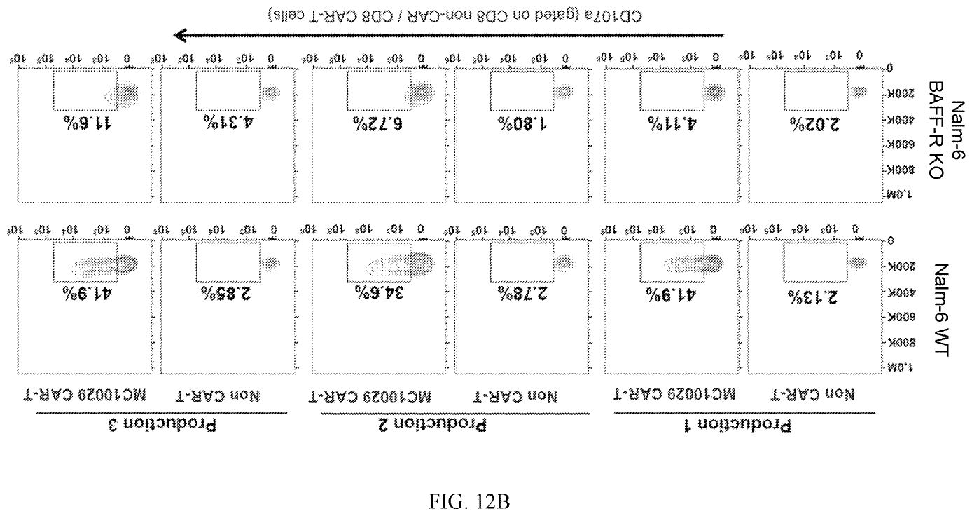

FIGS. 11A-11C show the characterization of MC10029 CAR-T cells used to target primary CLL tumor cells and that elicited the responses shown in FIG. 9B-9C. MC10029 CAR-T cells were generated from two healthy donors. The two batches of MC10029 CAR-T cells and non CAR-T cells that were engineered from T cells isolated from two healthy donors were shown to be identical as determined by viability, identity, and potency (FIG. 11A-11C) thus allowing for comparison of the two batches of MC10029 CAR-T cells in the in vitro assays.

Together, these results demonstrate that binders having two sets of three CDRs described herein (e.g., SEQ ID NOs:1-3 and 9-11) can bind to a BAFF-R polypeptide. Also as demonstrated herein, these binders can be engineered into molecules (e.g., antibodies, antigen binding fragments, antibody domains, CARs, cell engagers (e.g., BiTEs), or ADCs (e.g., antibody-radioconjugates)) that can be used to a treat a mammal (e.g., a human) having cancer (e.g., a B-cell cancer).

### Example 2: Exemplary BAFF-R Polypeptides

This Example depicts exemplary human BAFF-R sequences.

### Exemplary Human BAFF-R Polypeptide

(SEQ ID NO: 42)
MRRGPRSLRGRDAPAPTPCVPAECFDLLVRHCVACGLLRTPRPKPAGAS
SPAPRTALQPQESVGAGAGEAALPLPGLLFGAPALLGLALVLALVLVGL
VSWRRRQRRLRGASSAEAPDGDKDAPEPLDKVIILSPGISDATAPAWPP
PGEDPGTTPPGHSVPVPATELGSTELVTTKTAGPEQQ

### Nucleic Acid Encoding SEQ ID NO:42

(SEQ ID NO: 43)
ATGAGGCGAGGGCCCCGGAGCCTGCGGGGCAGGGACGCGCCAGCCCCCA
CGCCCTGCGTCCCGGCCGAGTGCTTCGACCTGCTGGTCCGCCACTGCGT
GGCCTGCGGGCTCCTGCGCACGCCGCGGCCGAAACCGGCCGGGGCCAGC
AGCCCTGCGCCCAGGACGGCGCTGCAGCCGCAGGAGTCGGTGGGCGCGG
GGGCCGGCGAGGCGGCGCTGCCCCTGCCCGGGCTGCTCTTTGGCGCCCC
CGCGCTGCTGGGCCTGGCACTGGTCCTGGCGCTGGTCCTGGTGGGTCTG
GTGAGCTGGAGGCGGCGACAGCGGCGGCTTCGCGGCGCGTCCTCCGCAG
AGGCCCCCGACGGAGACAAGGACGCCCCAGAGCCCCTGGACAAGGTCAT
CATTCTGTCTCCGGGAATCTCTGATGCCACAGCTCCTGCCTGGCCTCCT
CCTGGGGAAGACCCAGGAACCACCCCACCTGGCCACAGTGTCCCTGTGC
CAGCCACAGAGCTGGGCTCCACTGAACTGGTGACCACCAAGACGGCCGG
CCCTGAGCAACAATAG

### Example 3: Exemplary Variable Domains Having the Ability to Bind a BAFF-R Polypeptide

This Example provides the amino acid sequences of the heavy chain variable domain and the light chain variable domain of an antibody designated Clone #1 (also referred to as C21). The CDRs and framework sequences of each also are provided and delineated.

### Anti-BAFF-R Clone #1 (C21) Heavy Chain

### Heavy Chain Variable Domain:

(SEQ ID NO: 8)
DVQLVESGGGLVQPGGSRKLSCAASGFTFSSFGMHWVRQAPEKGLEWVA
YINSGSSTINYADTVKGRFTISRDNPKNTLFLQMTSLRSEDTAMYYCTI
YYYAMDYWGQGTSVTVSS

### Framework Region 1 of Heavy Chain Variable Domain:

(SEQ ID NO: 4)
DVQLVESGGGLVQPGGSRKLSCAAS

### CDR1 of Heavy Chain Variable Domain:

(SEQ ID NO: 1)
GFTFSSFG

### Framework Region 2 of Heavy Chain Variable Domain:

(SEQ ID NO: 5)
MHWVRQAPEKGLEWVAY

### CDR2 of Heavy Chain Variable Domain:

(SEQ ID NO: 2)
INSGSSTI

### Framework Region 3 of Heavy Chain Variable Domain:

(SEQ ID NO: 6)
NYADTVKGRFTISRDNPKNTLFLQMTSLRSEDTAMYYC

### CDR3 of Heavy Chain Variable Domain:

(SEQ ID NO: 3)
TIYYYAMDY

### Framework Region 4 of Heavy Chain Variable Domain:

(SEQ ID NO: 7)
WGQGTSVTVSS

### Anti-BAFF-R Clone #1 (C21) Light Chain

### Light Chain Variable Domain:

(SEQ ID NO: 16)
DIVMSQSPSSLTVSVGEKVSMSCKSSQTLLYSGNOKNYLAWYQQKPGQS
PKLLIYWASTRESGVPDRFAGSGSGTDFTLTISSVKAEDLAVYYCQQYY
IYPPWTFGGGTKLEIK

### Framework Region 1 of Light Chain Variable Domain:

(SEQ ID NO: 12)
DIVMSQSPSSLTVSVGEKVSMSCKSS

### CDR1 of Light Chain Variable Domain:

(SEQ ID NO: 9)
QTLLYSGNQKNY

### Framework Region 2 of Light Chain Variable Domain:

(SEQ ID NO: 13)
LAWYQQKPGQSPKLLIY

### CDR2 of Light Chain Variable Domain:

(SEQ ID NO: 10)
WAS

### Framework Region 3 of Light Chain Variable Domain:

(SEQ ID NO: 14)
TRESGVPDRFAGSGSGTDFTLTISSVKAEDLAVYYC

### CDR3 of Light Chain Variable Domain:

(SEQ ID NO: 11)
QQYYIYPPWT

### Framework Region 4 of Light Chain Variable Domain:

(SEQ ID NO: 15)
FGGGTKLEIK

### Example 4: Exemplary Fabs Having the Ability to Bind a BAFF-R Polypeptide

This Example provides the amino acid sequences of the heavy chain variable domain and the light chain variable domain of an antibody designated Clone #1 (also referred to as C21). The constant domains of each also are provided and delineated.

### Anti-BAFF-R Clone #1 (C21) Heavy Chain

Heavy Chain Variable Domain (without First Constant Domain):

(SEQ ID NO: 8)
DVQLVESGGGLVQPGGSRKLSCAASGFTFSSFGMHWVRQAPEKGLEWVA
YINSGSSTINYADTVKGRFTISRDNPKNTLFLQMTSLRSEDTAMYYCTI
YYYAMDYWGQGTSVTVSS

### Human IgG1 Heavy Chain Constant Domain 1 (CH1):

(SEQ ID NO: 46)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV

### Anti-BAFF-R Clone #1 (C21) Light Chain

Light Chain Variable Domain (without Kappa/Lambda Constant Domain):

(SEQ ID NO: 16)
DIVMSQSPSSLTVSVGEKVSMSCKSSQTLLYSGNQKNYLAWYQQKPGQS
PKLLIYWASTRESGVPDRFAGSGSGTDFTLTISSVKAEDLAVYYCQQYY
IYPPWTFGGGTKLEIK

### Example 5: Nucleic Acids Encoding Exemplary BAFF-R Binding Molecules

This Example provides the nucleic acid sequences encoding the indicated chains/domains of Clone #1 (C21).

### Clone #1

### Nucleic Acid Encoding SEQ ID NO:8 (Clone #1; Heavy Chain):

(SEQ ID NO: 44)
GATGTGCAGCTGGTGGAGTCTGGGGGAGGCTTAGTGCAGCCTGGAGGGT
CCCGGAAACTCTCCTGTGCAGCCTCTGGATTCACTTTCAGTAGCTTTGG
AATGCACTGGGTTCGTCAGGCTCCAGAGAAGGGGCTGGAGTGGGTCGCA
TACATTAATAGTGGCAGTAGTACCATTAACTATGCAGACACAGTGAAGG
GCCGATTCACCATCTCCAGAGACAATCCCAAGAACACCCTGTTCCTGCA
AATGACCAGTCTAAGGTCTGAGGACACGGCCATGTATTACTGTACAATC
TACTACTATGCTATGGACTACTGGGGTCAAGGAACCTCAGTCACCGTCT
CCTCA

### Nucleic Acid Encoding SEQ ID NO:16 (Clone #1; Light Chain):

(SEQ ID NO: 45)
GACATTGTGATGTCACAGTCTCCATCCTCCCTAACTGTGTCAGTTGGAG
AGAAGGTTTCTATGAGCTGCAAGTCCAGTCAGACCCTTTTATATAGTGG
CAATCAAAAGAACTACTTGGCCTGGTACCAGCAGAAACCAGGGCAGTCT
CCTAAACTGCTGATTTACTGGGCATCCACTAGGGAATCTGGGGTCCCTG
ATCGCTTCGCAGGCAGTGGATCTGGGACAGATTTCACTCTCACCATCAG
CAGTGTGAAGGCTGAAGACCTGGCAGTTTATTACTGTCAACAATATTAT
ATCTATCCTCCGTGGACGTTCGGTGGAGGCACCAAGCTGGAAATCAAA

### Example 6: Exemplary IgGs Having the Ability to Bind a BAFF-R Polypeptide

This Example provides the structure of an exemplary Ig and provides the amino acid and nucleic acid sequences of an exemplary hinge, CH2, and CH3 regions/domains.

### Exemplary Ig (e.g., IgG1) Structure

Heavy chain: Heavy Chain Variable Domain+CH1+Hinge+CH2+CH3
Light chain: Light Chain Variable Domain+Constant Light (Kappa or Lambda)

### Human Immunoglobulin G1 Hinge Region:

(SEQ ID NO: 47)
EPKSCDKTHTCPPCP
(SEQ ID NO: 48)
GAGCCCAAATCTTGTGACAAAACTCACACATGCCCACCGTGCCCA

### Human Immunoglobulin G1 Heavy Chain Constant Domains 2 and 3 (CH2-CH3):

(SEQ ID NO: 49)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCS
VMHEALHNHYTQKSLSLSPGK
(SEQ ID NO: 50)
GCACCTGAACTCCTGGGGGGACCGTCAGTCTTCCTCTTCCCCCCAAAAC
CCAAGGACACCCTCATGATCTCCCGGACCCCTGAGGTCACATGCGTGGT
GGTGGACGTGAGCCACGAAGACCCTGAGGTCAAGTTCAACTGGTACGTG
GACGGCGTGGAGGTGCATAATGCCAAGACAAAGCCGCGGGAGGAGCAGT
ACAACAGCACGTACCGTGTGGTCAGCGTCCTCACCGTCCTGCACCAGGA
CTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAG
AACCACAGGTGTACACCCTGCCCCCATCCCGGGATGAGCTGACCAAGAA
CCAGGTCAGCCTGACCTGCCTGGTCAAAGGCTTCTATCCCAGCGACATC
GCCGTGGAGTGGGAGAGCAATGGGCAGCCGGAGAACAACTACAAGACCA
CGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCC
GTGATGCATGAGGCTCTGCACAACCACTACACGCAGAAGAGCCTCTCCC
TGTCTCCGGGTAAA

### Example 7: Exemplary scFvs Having the Ability to Bind a BAFF-R Polypeptide

This Example provides structures of exemplary scFv's, as well as the amino acid sequences of an exemplary heavy chain variable domain and an exemplary light chain variable domain of an exemplary scFv. The CDRs and framework sequences of each also are delineated. An exemplary linker amino acid sequence such as a linker amino acid sequence set forth in Example 10 can be used to link the heavy chain variable domain and the light chain variable domain together to form a scFv. Structures of exemplary scFv's including the amino acid and nucleic acid sequences with the linker, CDRs, and framework sequences delineated are shown.

Exemplary scFv Structure:
Heavy Chain Variable Domain/Region+Linker+Light Chain Variable Domain/Region
   Exemplary scFv Structure:
Light Chain Variable Domain/Region+Linker+Heavy Chain Variable Domain/Region
   Exemplary scFv Heavy Chain Variable Domain Structure:

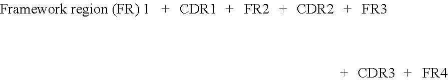

### Exemplary Variable Heavy Region FR1 Sequences:

(SEQ ID NO: 4)
DVQLVESGGGLVQPGGSRKLSCAAS

### Exemplary Variable Heavy Region FR2 Sequences:

(SEQ ID NO: 5)
MHWVRQAPEKGLEWVAY

### Exemplary Variable Heavy Region FR3 Sequences:

(SEQ ID NO: 6)
NYADTVKGRFTISRDNPKNTLFLQMTSLRSEDTAMYYC

### Exemplary Variable Heavy Region FR4 Sequences:

(SEQ ID NO: 7)
WGQGTSVTVSS

Exemplary scFv Light Chain Variable Domain Structure:

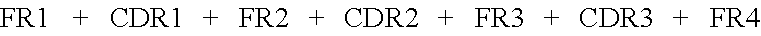

### Exemplary Variable Light Region FR1 Sequences:

(SEQ ID NO: 12)
DIVMSQSPSSLTVSVGEKVSMSCKSS

### Exemplary Variable Light Region FR2 Sequences:

(SEQ ID NO: 13)
LAWYQQKPGQSPKLLIY

### Exemplary Variable Light Region FR3 Sequences:

(SEQ ID NO: 14)
TRESGVPDRFAGSGSGTDFTLTISSVKAEDLAVYYC

### Exemplary Variable Light Region FR4 Sequences:

(SEQ ID NO: 15)
FGGGTKLEIK

Exemplary scFv Structure
Variable Light Region Using CDRs of Clone #1+Linker (e.g., (G₄S)₅ Linker)+Variable Heavy Region Using CDRs of Clone #1:

(SEQ ID NO: 17)
DIVMSQSPSSLTVSVGEKVSMSCKSSQTLLYSGNQKNYLAWYQQKPGQSP
KLLIYWASTRESGVPDRFAGSGSGTDFTLTISSVKAEDLAVYYCQQYYIY
PPWTFGGGTKLEIKGGGGSGGGGSGGGGSGGGGSGGGGSDVQLVESGGGL
VQPGGSRKLSCAASGFTFSSFGMHWVRQAPEKGLEWVAYINSGSSTINYA
DTVKGRFTISRDNPKNTLFLQMTSLRSEDTAMYYCTIYYYAMDYWGQGTS
VTVSS
(SEQ ID NO: 18)
GACATTGTGATGTCACAGTCTCCATCCTCCCTAACTGTGTCAGTTGGAGA
GAAGGTTTCTATGAGCTGCAAGTCCAGTCAGACCCTTTTATATAGTGGCA
ATCAAAAGAACTACTTGGCCTGGTACCAGCAGAAACCAGGGCAGTCTCCT
AAACTGCTGATTTACTGGGCATCCACTAGGGAATCTGGGGTCCCTGATCG
CTTCGCAGGCAGTGGATCTGGGACAGATTTCACTCTCACCATCAGCAGTG
TGAAGGCTGAAGACCTGGCAGTTTATTACTGTCAACAATATTATATCTAT
CCTCCGTGGACGTTCGGTGGAGGCACCAAGCTGGAAATCAAAGGAGGGGG
TGGCAGCGGTGGCGGGGGAAGTGGCGGTGGAGGGTCGGGGGGAGGCGGTT
CCGGGGGTGGAGGCTCAGATGTGCAGCTGGTGGAGTCTGGGGGAGGCTTA
GTGCAGCCTGGAGGGTCCCGGAAACTCTCCTGTGCAGCCTCTGGATTCAC
TTTCAGTAGCTTTGGAATGCACTGGGTTCGTCAGGCTCCAGAGAAGGGGC
TGGAGTGGGTCGCATACATTAATAGTGGCAGTAGTACCATTAACTATGCA
GACACAGTGAAGGGCCGATTCACCATCTCCAGAGACAATCCCAAGAACAC
CCTGTTCCTGCAAATGACCAGTCTAAGGTCTGAGGACACGGCCATGTATT
ACTGTACAATCTACTACTATGCTATGGACTACTGGGGTCAAGGAACCTCA
GTCACCGTCTCCTCA

Exemplary scFv Structure
Variable Light Region Using CDRs of Clone #1+Linker (e.g., (G₄S)₃ Linker)+Variable Heavy Region Using CDRs of Clone #1:

(SEQ ID NO: 19)
DIVMSQSPSSLTVSVGEKVSMSCKSSQTLLYSGNQKNYLAWYQQKPGQSP
KLLIYWASTRESGVPDRFAGSGSGTDFTLTISSVKAEDLAVYYCQQYYIY
PPWTFGGGTKLEIKGGGGSGGGGSGGGGSDVQLVESGGGLVQPGGSRKLS
CAASGFTFSSFGMHWVRQAPEKGLEWVAYINSGSSTINYADTVKGRFTIS
RDNPKNTLFLQMTSLRSEDTAMYYCTIYYYAMDYWGQGTSVTVSS
(SEQ ID NO: 20)
GACATTGTGATGTCACAGTCTCCATCCTCCCTAACTGTGTCAGTTGGAGA
GAAGGTTTCTATGAGCTGCAAGTCCAGTCAGACCCTTTTATATAGTGGCA
ATCAAAAGAACTACTTGGCCTGGTACCAGCAGAAACCAGGGCAGTCTCCT
AAACTGCTGATTTACTGGGCATCCACTAGGGAATCTGGGGTCCCTGATCG
CTTCGCAGGCAGTGGATCTGGGACAGATTTCACTCTCACCATCAGCAGTG
TGAAGGCTGAAGACCTGGCAGTTTATTACTGTCAACAATATTATATCTAT
CCTCCGTGGACGTTCGGTGGAGGCACCAAGCTGGAAATCAAAGGAGGGGG
TGGCAGCGGTGGCGGGGGAAGTGGCGGTGGAGGGTCGGATGTGCAGCTGG
TGGAGTCTGGGGGAGGCTTAGTGCAGCCTGGAGGGTCCCGGAAACTCTCC
TGTGCAGCCTCTGGATTCACTTTCAGTAGCTTTGGAATGCACTGGGTTCG
TCAGGCTCCAGAGAAGGGGCTGGAGTGGGTCGCATACATTAATAGTGGCA
GTAGTACCATTAACTATGCAGACACAGTGAAGGGCCGATTCACCATCTCC
AGAGACAATCCCAAGAACACCCTGTTCCTGCAAATGACCAGTCTAAGGTC
TGAGGACACGGCCATGTATTACTGTACAATCTACTACTATGCTATGGACT
ACTGGGGTCAAGGAACCTCAGTCACCGTCTCCTCA

Exemplary scFv Structure
Variable Heavy Region Using CDRs of Clone #1+Linker (e.g., (G₄S)₅ Linker)+Variable Light Region Using CDRs of Clone #1:

(SEQ ID NO: 21)
DVQLVESGGGLVQPGGSRKLSCAASGFTFSSFGMHWVRQAPEKGLEWVAY
INSGSSTINYADTVKGRFTISRDNPKNTLFLQMTSLRSEDTAMYYCTIYY
YAMDYWGQGTSVTVSSGGGGSGGGGSGGGGSGGGGSGGGGSDIVMSQSPS
SLTVSVGEKVSMSCKSSQTLLYSGNQKNYLAWYQQKPGQSPKLLIYWAST
RESGVPDRFAGSGSGTDFTLTISSVKAEDLAVYYCQQYYIYPPWTFGGGT
KLEIK
(SEQ ID NO: 22)
GATGTGCAGCTGGTGGAGTCTGGGGGAGGCTTAGTGCAGCCTGGAGGGTC
CCGGAAACTCTCCTGTGCAGCCTCTGGATTCACTTTCAGTAGCTTTGGAA
TGCACTGGGTTCGTCAGGCTCCAGAGAAGGGGCTGGAGTGGGTCGCATAC
ATTAATAGTGGCAGTAGTACCATTAACTATGCAGACACAGTGAAGGGCCG
ATTCACCATCTCCAGAGACAATCCCAAGAACACCCTGTTCCTGCAAATGA
CCAGTCTAAGGTCTGAGGACACGGCCATGTATTACTGTACAATCTACTAC
TATGCTATGGACTACTGGGGTCAAGGAACCTCAGTCACCGTCTCCTCAGG
AGGGGGTGGCAGCGGTGGCGGGGGAAGTGGCGGTGGAGGGTCGGGGGGAG
GCGGTTCCGGGGGTGGAGGCTCAGACATTGTGATGTCACAGTCTCCATCC
TCCCTAACTGTGTCAGTTGGAGAGAAGGTTTCTATGAGCTGCAAGTCCAG
TCAGACCCTTTTATATAGTGGCAATCAAAAGAACTACTTGGCCTGGTACC
AGCAGAAACCAGGGCAGTCTCCTAAACTGCTGATTTACTGGGCATCCACT
AGGGAATCTGGGGTCCCTGATCGCTTCGCAGGCAGTGGATCTGGGACAGA
TTTCACTCTCACCATCAGCAGTGTGAAGGCTGAAGACCTGGCAGTTTATT
ACTGTCAACAATATTATATCTATCCTCCGTGGACGTTCGGTGGAGGCACC
AAGCTGGAAATCAAA

Exemplary scFv Structure
Variable Heavy Region Using CDRs of Clone #1+Linker (e.g., (G₄S)₃ Linker)+Variable Light Region Using CDRs of Clone #1:

(SEQ ID NO: 23)
DVQLVESGGGLVQPGGSRKLSCAASGFTFSSFGMHWVRQAPEKGLEWVAY
INSGSSTINYADTVKGRFTISRDNPKNTLFLQMTSLRSEDTAMYYCTIYY
YAMDYWGQGTSVTVSSGGGGSGGGGSGGGGSDIVMSQSPSSLTVSVGEKV
SMSCKSSQTLLYSGNQKNYLAWYQQKPGQSPKLLIYWASTRESGVPDRFA
GSGSGTDFTLTISSVKAEDLAVYYCOQYYIYPPWTFGGGTKLEIK
(SEQ ID NO: 24)
GATGTGCAGCTGGTGGAGTCTGGGGGAGGCTTAGTGCAGCCTGGAGGGTC
CCGGAAACTCTCCTGTGCAGCCTCTGGATTCACTTTCAGTAGCTTTGGAA
TGCACTGGGTTCGTCAGGCTCCAGAGAAGGGGCTGGAGTGGGTCGCATAC
ATTAATAGTGGCAGTAGTACCATTAACTATGCAGACACAGTGAAGGGCCG
ATTCACCATCTCCAGAGACAATCCCAAGAACACCCTGTTCCTGCAAATGA
CCAGTCTAAGGTCTGAGGACACGGCCATGTATTACTGTACAATCTACTAC
TATGCTATGGACTACTGGGGTCAAGGAACCTCAGTCACCGTCTCCTCAGG
AGGGGGTGGCAGCGGTGGCGGGGGAAGTGGCGGTGGAGGGTCGGACATTG
TGATGTCACAGTCTCCATCCTCCCTAACTGTGTCAGTTGGAGAGAAGGTT
TCTATGAGCTGCAAGTCCAGTCAGACCCTTTTATATAGTGGCAATCAAAA
GAACTACTTGGCCTGGTACCAGCAGAAACCAGGGCAGTCTCCTAAACTGC
TGATTTACTGGGCATCCACTAGGGAATCTGGGGTCCCTGATCGCTTCGCA
GGCAGTGGATCTGGGACAGATTTCACTCTCACCATCAGCAGTGTGAAGGC
TGAAGACCTGGCAGTTTATTACTGTCAACAATATTATATCTATCCTCCGT
GGACGTTCGGTGGAGGCACCAAGCTGGAAATCAAA

### Example 8: Exemplary Heavy Chain Framework Region Sequences

This Example provides exemplary framework region amino acid sequences that can be used in a heavy chain variable domain/region (e.g., a heavy chain variable domain/region that can be used to form a scFv.

### Exemplary Variable Heavy Region FR1 Sequences:

(SEQ ID NO: 4)
DVQLVESGGGLVQPGGSRKLSCAAS

### Exemplary Variable Heavy Region FR2 Sequences:

(SEQ ID NO: 5)
MHWVRQAPEKGLEWVAY

### Exemplary Variable Heavy Region FR3 Sequences:

(SEQ ID NO: 6)
NYADTVKGRFTISRDNPKNTLFLQMTSLRSEDTAMYYC

### Exemplary Variable Heavy Region FR4 Sequences:

(SEQ ID NO: 7)
WGQGTSVTVSS

### Example 9: Exemplary Light Chain Framework Region Sequences

This Example provides exemplary framework region amino acid sequences that can be used in a light chain variable domain/region (e.g., a light chain variable domain/region that can be used to form a scFv.

Exemplary Variable Light Region FR1 sequences:

(SEQ ID NO: 12)
DIVMSQSPSSLTVSVGEKVSMSCKSS

### Exemplary Variable Light Region FR2 Sequences:

(SEQ ID NO: 13)
LAWYQQKPGQSPKLLIY

### Exemplary Variable Light Region FR3 Sequences:

(SEQ ID NO: 14)
TRESGVPDRFAGSGSGTDFTLTISSVKAEDLAVYYC

### Exemplary Variable Light Region FR4 Sequences:

(SEQ ID NO: 15)
FGGGTKLEIK

### Example 10: Exemplary Linkers

This Example provides exemplary linker amino acid sequences that can be used to link a heavy chain variable domain and a light chain variable domain together to form a scFv. These linker sequences also can be used to create CARs and cell engagers.

(SEQ ID NO: 25)
GGGGSGGGGSGGGGS
(SEQ ID NO: 26)
GGGGSGGGGSGGGGSGGGGSGGGGS

### Nucleic Acid Encoding SEQ ID NO:25

(SEQ ID NO: 27)
GGTGGAGGCGGTTCAGGTGGCGGCGGTTCGGGCGGTGGCGGCTCT

### Example 11: Exemplary Signal Peptides

This Example provides the amino acid sequences of exemplary signal peptides that can be used to design a CAR.

### Exemplary Human GMCSF-Derived Signal Peptide:

(SEQ ID NO: 28)
MLLLVTSLLLCELPHPAFLLIP

### Nucleic Acid Encoding SEQ ID NO:28

(SEQ ID NO: 29)
ATGCTGCTGCTCGTGACATCTCTGCTGCTGTGCGAGCTGCCCCAC
CCCGCCTTTCTGCTGATTCCT

### Example 12: Exemplary Hinges

This Example provides the amino acid sequences of exemplary hinges that can be used to design a CAR.

### Exemplary Human IgG4-Derived Hinge:

(SEQ ID NO: 30)
ESKYGPPCPPCPAPEFEGGPSVFLFPPKPKDTLMISRTPEVTCVV
VDVSQEDPEVQFNWYVDGVEVHNAKTKPREEQFQSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLGK

### Nucleic Acid Encoding SEQ ID NO:30:

(SEQ ID NO: 31)
GAGTCTAAGTACGGCCCTCCCTGCCCCCCCTGCCCAGCCCCTGAA
TTTGAGGGCGGACCCAGCGTGTTCCTGTTCCCCCCAAAGCCCAAG
GACACCCTGATGATCAGCCGGACCCCCGAGGTAACCTGCGTGGTG
GTGGACGTGTCCCAGGAAGATCCCGAGGTCCAGTTCAATTGGTAC
GTGGACGGCGTGGAAGTGCACAATGCCAAGACCAAGCCCAGAGAG
GAACAGTTCCAAAGCACCTACCGGGTGGTGTCCGTGCTGACCGTG
CTGCACCAGGACTGGCTGAACGGCAAAGAGTACAAGTGCAAGGTG
TCCAACAAGGGCCTGCCCAGCAGCATCGAGAAAACCATCAGCAAG
GCCAAGGGCCAGCCCCGCGAGCCCCAGGTGTACACACTGCCCCCC
AGCCAGGAAGAGATGACCAAGAACCAGGTGTCCCTGACCTGCCTG
GTCAAGGGCTTCTACCCCAGCGATATCGCCGTGGAATGGGAGAGC
AACGGCCAGCCCGAGAACAACTACAAGACCACCCCCCCTGTGCTG
GACAGCGACGGCAGCTTCTTCCTGTACTCCCGGCTGACCGTGGAC
AAGAGCCGGTGGCAGGAAGGCAACGTCTTCAGCTGCAGCGTGATG
CACGAGGCCCTGCACAACCACTACACCCAGAAGTCTCTGAGCCTG
AGCCTGGGCAAG

### Example 13: Exemplary Transmembrane Domains

This Example provides the amino acid sequences of exemplary transmembrane domains that can be used to design a CAR.

### Exemplary Human CD28 Transmembrane Domain:

(SEQ ID NO: 32)
MFWVLVVVGGVLACYSLLVTVAFIIFWV

### Nucleic Acid Encoding SEQ ID NO:32

(SEQ ID NO: 33)
ATGTTCTGGGTGCTGGTGGTGGTGGGCGGGGTGCTGGCCTGCTAC
AGCCTGCTGGTGACAGTGGCCTTCATCATCTTTTGGGTG

### Example 14: Exemplary Intracellular Signaling Domains

This Example provides the amino acid sequences of exemplary intracellular signaling domains that can be used to design a CAR.

### Exemplary Human CD28 Intracellular Signaling Domain (CD28QQ):

(SEQ ID NO: 34)
RSKRSRGGHSDYMNMTPRRPGPTRKHYQPYAPPRDFAAYRS

### Nucleic Acid Encoding SEQ ID NO:34:

(SEQ ID NO: 35)
CGGAGCAAGCGGAGCAGAGGCGGCCACAGCGACTACATGAACATG
ACCCCCAGACGGCCTGGCCCCACCCGGAAGCACTACCAGCCCTAC
GCCCCACCCAGGGACTTTGCCGCCTACCGGTCC

### Exemplary Human CD3ζ Intracellular Signaling Domain:

(SEQ ID NO: 36)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

Nucleic acid encoding SEQ ID NO:36:

(SEQ ID NO: 37)
AGAGTGAAGTTCAGCCGGTCCGCCGACGCCCCTGCCTACCAGCAG
GGCCAGAACCAGCTGTACAACGAGCTGAACCTGGGCAGGCGGGAG
GAATACGACGTGCTGGACAAGCGGAGAGGCCGGGACCCTGAGATG
GGCGGCAAGCCCAGGCGGAAGAACCCTCAGGAAGGCCTGTATAAC
GAACTGCAGAAAGACAAGATGGCCGAGGCCTACAGCGAGATCGGC
ATGAAGGGCGAGCGGCGGAGGGGCAAGGGCCACGACGGCCTGTAC
CAGGGCCTGAGCACCGCCACCAAGGATACCTACGACGCCCTGCAC
ATGCAGGCCCTGCCCCCAAGG

### Example 15: Exemplary Detectable Markers

This Example provides the amino acid sequences of exemplary detectable markers that can be used to design a CAR.

Exemplary tEGFR Polypeptide:

(SEQ ID NO: 38)
MLLLVTSLLLCELPHPAFLLIPRKVCNGIGIGEFKDSLSINATNI
KHFKNCTSISGDLHILPVAFRGDSFTHTPPLDPQELDILKTVKEI
TGFLLIQAWPENRTDLHAFENLEIIRGRTKQHGQFSLAVVSLNIT
SLGLRSLKEISDGDVIISGNKNLCYANTINWKKLFGTSGQKTKII
SNRGENSCKATGQVCHALCSPEGCWGPEPRDCVSCRNVSRGRECV
DKCNLLEGEPREFVENSECIQCHPECLPQAMNITCTGRGPDNCIQ
CAHYIDGPHCVKTCPAGVMGENNTLVWKYADAGHVCHLCHPNCTY
GCTGPGLEGCPTNGPKIPSIATGMVGALLLLLVVALGIGLFM

### Nucleic Acid Encoding SEQ ID NO:38:

(SEQ ID NO: 39)
ATGCTTCTCCTGGTGACAAGCCTTCTGCTCTGTGAGTTACCACAC
CCAGCATTCCTCCTGATCCCACGCAAAGTGTGTAACGGAATAGGT
ATTGGTGAATTTAAAGACTCACTCTCCATAAATGCTACGAATATT
AAACACTTCAAAAACTGCACCTCCATCAGTGGCGATCTCCACATC
CTGCCGGTGGCATTTAGGGGTGACTCCTTCACACATACTCCTCCT
CTGGATCCACAGGAACTGGATATTCTGAAAACCGTAAAGGAAATC
ACAGGGTTTTTGCTGATTCAGGCTTGGCCTGAAAACAGGACGGAC
CTCCATGCCTTTGAGAACCTAGAAATCATACGCGGCAGGACCAAG
CAACATGGTCAGTTTTCTCTTGCAGTCGTCAGCCTGAACATAACA
TCCTTGGGATTACGCTCCCTCAAGGAGATAAGTGATGGAGATGTG
ATAATTTCAGGAAACAAAAATTTGTGCTATGCAAATACAATAAAC
TGGAAAAAACTGTTTGGGACCTCCGGTCAGAAAACCAAAATTATA
AGCAACAGAGGTGAAAACAGCTGCAAGGCCACAGGCCAGGTCTGC
CATGCCTTGTGCTCCCCCGAGGGCTGCTGGGGCCCGGAGCCCAGG
GACTGCGTCTCTTGCCGGAATGTCAGCCGAGGCAGGGAATGCGTG
GACAAGTGCAACCTTCTGGAGGGTGAGCCAAGGGAGTTTGTGGAG
AACTCTGAGTGCATACAGTGCCACCCAGAGTGCCTGCCTCAGGCC
ATGAACATCACCTGCACAGGACGGGGACCAGACAACTGTATCCAG
TGTGCCCACTACATTGACGGCCCCCACTGCGTCAAGACCTGCCCG
GCAGGAGTCATGGGAGAAAACAACACCCTGGTCTGGAAGTACGCA
GACGCCGGCCATGTGTGCCACCTGTGCCATCCAAACTGCACCTAC
GGATGCACTGGGCCAGGTCTTGAAGGCTGTCCAACGAATGGGCCT
AAGATCCCGTCCATCGCCACTGGGATGGTGGGGGCCCTCCTCTTG
CTGCTGGTGGTGGCCCTGGGGATCGGCCTCTTCATG

### Example 16: Exemplary CARs Having the Ability to Bind a BAFF-R Polypeptide

This Example provides an amino acid sequence of a CAR (CAR #1) designed to include a scFv created using the CDRs of the Clone #1 Fab and a nucleic acid sequence encoding that CAR. The various components of this CAR (e.g., domains and linkers) are provided and delineated.

### CAR Designed Using CDRs of Clone #1:

signal peptide+scFv of Example 7+hinge/linker+CD28 Transmembrane domain+CD28 Intracellular Signaling Domain+CD3ζ Intracellular Signaling Domain+T2A+tEGFR

(SEQ ID NO: 40)
MLLLVTSLLLCELPHPAFLLIPDVQLVESGGGLVQPGGSRKLSCA
ASGFTFSSFGMHWVRQAPEKGLEWVAYINSGSSTINYADTVKGRF
TISRDNPKNTLFLQMTSLRSEDTAMYYCTIYYYAMDYWGQGTSVT
VSSGGGGSGGGGSGGGGSDIVMSQSPSSLTVSVGEKVSMSCKSSQ
TLLYSGNQKNYLAWYQQKPGQSPKLLIYWASTRESGVPDRFAGSG
SGTDFTLTISSVKAEDLAVYYCQQYYIYPPWTFGGGTKLEIKESK
YGPPCPPCPAPEFEGGPSVFLFPPKPKDTLMISRTPEVTCVVVDV
SQEDPEVQFNWYVDGVEVHNAKTKPREEQFQSTYRVVSVLTVLHQ
DWLNGKEYKCKVSNKGLPSSIEKTISKAKGQPREPQVYTLPPSQE
EMTKNQVSLTCLVKGFYPSDIAVEWESNGQPENNYKTTPPVLDSD
GSFFLYSRLTVDKSRWQEGNVFSCSVMHEALHNHYTQKSLSLSLG
KMFWVLVVVGGVLACYSLLVTVAFIIFWVRSKRSRGGHSDYMNMT
PRRPGPTRKHYQPYAPPRDFAAYRSRVKFSRSADAPAYQQGQNQL
YNELNLGRREEYDVLDKRRGRDPEMGGKPRRKNPQEGLYNELQKD
KMAEAYSEIGMKGERRRGKGHDGLYQGLSTATKDTYDALHMQALP
PRLEGGGEGRGSLLTCGDVEENPGPRMLLLVTSLLLCELPHPAFL
LIPRKVCNGIGIGEFKDSLSINATNIKHFKNCTSISGDLHILPVA
FRGDSFTHTPPLDPQELDILKTVKEITGFLLIQAWPENRTDLHAF
ENLEIIRGRTKQHGQFSLAVVSLNITSLGLRSLKEISDGDVIISG
NKNLCYANTINWKKLFGTSGQKTKIISNRGENSCKATGQVCHALC
SPEGCWGPEPRDCVSCRNVSRGRECVDKCNLLEGEPREFVENSEC
IQCHPECLPQAMNITCTGRGPDNCIQCAHYIDGPHCVKTCPAGVM
GENNTLVWKYADAGHVCHLCHPNCTYGCTGPGLEGCPTNGPKIPS
IATGMVGALLLLLVVALGIGLFM

### Nucleic Acid Sequence Encoding SEQ ID NO:40:

(SEQ ID NO: 41)
ATGCTGCTGCTCGTGACATCTCTGCTGCTGTGCGAGCTGCCCCAC
CCCGCCTTTCTGCTGATTCCTGATGTGCAGCTGGTGGAGTCTGGG
GGAGGCTTAGTGCAGCCTGGAGGGTCCCGGAAACTCTCCTGTGCA
GCCTCTGGATTCACTTTCAGTAGCTTTGGAATGCACTGGGTTCGT
CAGGCTCCAGAGAAGGGGCTGGAGTGGGTCGCATACATTAATAGT
GGCAGTAGTACCATTAACTATGCAGACACAGTGAAGGGCCGATTC
ACCATCTCCAGAGACAATCCCAAGAACACCCTGTTCCTGCAAATG
ACCAGTCTAAGGTCTGAGGACACGGCCATGTATTACTGTACAATC
TACTACTATGCTATGGACTACTGGGGTCAAGGAACCTCAGTCACC
GTCTCCTCAGGTGGAGGCGGTTCAGGTGGCGGCGGTTCGGGCGGT
GGCGGCTCTGACATTGTGATGTCACAGTCTCCATCCTCCCTAACT
GTGTCAGTTGGAGAGAAGGTTTCTATGAGCTGCAAGTCCAGTCAG
ACCCTTTTATATAGTGGCAATCAAAAGAACTACTTGGCCTGGTAC
CAGCAGAAACCAGGGCAGTCTCCTAAACTGCTGATTTACTGGGCA
TCCACTAGGGAATCTGGGGTCCCTGATCGCTTCGCAGGCAGTGGA
TCTGGGACAGATTTCACTCTCACCATCAGCAGTGTGAAGGCTGAA
GACCTGGCAGTTTATTACTGTCAACAATATTATATCTATCCTCCG
TGGACGTTCGGTGGAGGCACCAAGCTGGAAATCAAAGAGTCTAAG
TACGGCCCTCCCTGCCCCCCCTGCCCAGCCCCTGAATTTGAGGGC
GGACCCAGCGTGTTCCTGTTCCCCCCAAAGCCCAAGGACACCCTG
ATGATCAGCCGGACCCCCGAGGTAACCTGCGTGGTGGTGGACGTG
TCCCAGGAAGATCCCGAGGTCCAGTTCAATTGGTACGTGGACGGC
GTGGAAGTGCACAATGCCAAGACCAAGCCCAGAGAGGAACAGTTC
CAAAGCACCTACCGGGTGGTGTCCGTGCTGACCGTGCTGCACCAG
GACTGGCTGAACGGCAAAGAGTACAAGTGCAAGGTGTCCAACAAG
GGCCTGCCCAGCAGCATCGAGAAAACCATCAGCAAGGCCAAGGGC
CAGCCCCGCGAGCCCCAGGTGTACACACTGCCCCCCAGCCAGGAA
GAGATGACCAAGAACCAGGTGTCCCTGACCTGCCTGGTCAAGGGC
TTCTACCCCAGCGATATCGCCGTGGAATGGGAGAGCAACGGCCAG
CCCGAGAACAACTACAAGACCACCCCCCCTGTGCTGGACAGCGAC
GGCAGCTTCTTCCTGTACTCCCGGCTGACCGTGGACAAGAGCCGG
TGGCAGGAAGGCAACGTCTTCAGCTGCAGCGTGATGCACGAGGCC
CTGCACAACCACTACACCCAGAAGTCTCTGAGCCTGAGCCTGGGC
AAGATGTTCTGGGTGCTGGTGGTGGTGGGCGGGGTGCTGGCCTGC
TACAGCCTGCTGGTGACAGTGGCCTTCATCATCTTTTGGGTGCGG
AGCAAGCGGAGCAGAGGCGGCCACAGCGACTACATGAACATGACC
CCCAGACGGCCTGGCCCCACCCGGAAGCACTACCAGCCCTACGCC
CCACCCAGGGACTTTGCCGCCTACCGGTCCAGAGTGAAGTTCAGC
CGGTCCGCCGACGCCCCTGCCTACCAGCAGGGCCAGAACCAGCTG
TACAACGAGCTGAACCTGGGCAGGCGGGAGGAATACGACGTGCTG
GACAAGCGGAGAGGCCGGGACCCTGAGATGGGCGGCAAGCCCAGG
CGGAAGAACCCTCAGGAAGGCCTGTATAACGAACTGCAGAAAGAC
AAGATGGCCGAGGCCTACAGCGAGATCGGCATGAAGGGCGAGCGG
CGGAGGGGCAAGGGCCACGACGGCCTGTACCAGGGCCTGAGCACC
GCCACCAAGGATACCTACGACGCCCTGCACATGCAGGCCCTGCCC
CCAAGGCTCGAGGGCGGCGGAGAGGGCAGAGGAAGTCTTCTAACA
TGCGGTGACGTGGAGGAGAATCCCGGCCCTAGGATGCTTCTCCTG
GTGACAAGCCTTCTGCTCTGTGAGTTACCACACCCAGCATTCCTC
CTGATCCCACGCAAAGTGTGTAACGGAATAGGTATTGGTGAATTT
AAAGACTCACTCTCCATAAATGCTACGAATATTAAACACTTCAAA
AACTGCACCTCCATCAGTGGCGATCTCCACATCCTGCCGGTGGCA
TTTAGGGGTGACTCCTTCACACATACTCCTCCTCTGGATCCACAG
GAACTGGATATTCTGAAAACCGTAAAGGAAATCACAGGGTTTTTG
CTGATTCAGGCTTGGCCTGAAAACAGGACGGACCTCCATGCCTTT
GAGAACCTAGAAATCATACGCGGCAGGACCAAGCAACATGGTCAG
TTTTCTCTTGCAGTCGTCAGCCTGAACATAACATCCTTGGGATTA
CGCTCCCTCAAGGAGATAAGTGATGGAGATGTGATAATTTCAGGA
AACAAAAATTTGTGCTATGCAAATACAATAAACTGGAAAAAACTG
TTTGGGACCTCCGGTCAGAAAACCAAAATTATAAGCAACAGAGGT
GAAAACAGCTGCAAGGCCACAGGCCAGGTCTGCCATGCCTTGTGC
TCCCCCGAGGGCTGCTGGGGCCCGGAGCCCAGGGACTGCGTCTCT
TGCCGGAATGTCAGCCGAGGCAGGGAATGCGTGGACAAGTGCAAC
CTTCTGGAGGGTGAGCCAAGGGAGTTTGTGGAGAACTCTGAGTGC
ATACAGTGCCACCCAGAGTGCCTGCCTCAGGCCATGAACATCACC
TGCACAGGACGGGGACCAGACAACTGTATCCAGTGTGCCCACTAC
ATTGACGGCCCCCACTGCGTCAAGACCTGCCCGGCAGGAGTCATG
GGAGAAAACAACACCCTGGTCTGGAAGTACGCAGACGCCGGCCAT
GTGTGCCACCTGTGCCATCCAAACTGCACCTACGGATGCACTGGG
CCAGGTCTTGAAGGCTGTCCAACGAATGGGCCTAAGATCCCGTCC
ATCGCCACTGGGATGGTGGGGGCCCTCCTCTTGCTGCTGGTGGTG
GCCCTGGGGATCGGCCTCTTCATGTGA

### Example 17: BAFF-R CAR-T Therapy Targeting B-Cell Lymphoid Malignancies

The results in this Example re-present and expand on at least some of the results provided in other Examples.

### Materials and Methods

### Cell Lines

The cell lines of Nalm-6, MEC-1, and Z-138 were purchased from Deutsche Sammlung von Mikroorganismen und Zellkulturen GmbH (Braunschweig, Germany); Jurkat and 293FT cell lines were obtained from Thermo Fisher Scientific (Waltham, Massachusetts, USA) and American Type Culture Collection (Manassas, Virginia, USA), respectively. Cells were maintained in either 90% RPMI 1640, Iscove's MDM, or 90% Dulbecco's Modified Eagle Medium (Thermo Fisher) supplemented with 10% heat-inactivated fetal bovine serum (Thermo Fisher). Cell lines were authenticated by flow cytometry. The following antigen knock out cell lines were generated: BAFF-R KO Nalm-6, CD19 KO Nalm-6, CD19 KO Z-138, and CD19 KO MEC-1. Luciferase expressing human cell lines were generated for in vivo experiments, as described in Qin et al., Science Translational Medicine, 11(511) 2019.

PBMCs and Tn/Mem Isolation from Health Donors' Blood Samples

Peripheral blood mononuclear cells (PBMCs) from healthy volunteer donors were isolated via leukapheresis using leukocyte reduction system (LRS) cones as described in Dietz et al., Transfusion 46:2083-2089 (2006). For the generation of CAR-T cells, naive and memory T cell (Tn/mem) populations were isolated from PBMCs in a three-step procedure by negative selection of both CD14 and CD25 and positive selection of CD62L, using CD14, CD25, and CD62L microbeads, per the manufacturer protocol (Miltenyi Biotech, Germany).

Isolation of T Cells and B-Cells from Blood Samples from Subject with CLL

Peripheral blood samples from subjects with CLL were procured per a biorepository protocol. All subjects provided written informed consent. For primary cell analysis, 50 mL of blood was collected from subjects with a diagnosis of CLL. For CAR-T cell generation from CLL subjects, the T cells were isolated using the Pan T cell isolation kit (Miltenyi Biotec, Germany), following the instructions provided. The B-cells were isolated using the EasySep™ Direct Human B-cell Isolation Kit (Stemcell Technologies, Vancouver, Canada), per the manufacturer protocol.

### CAR-T Cell Production

A second-generation BAFF-R-CAR was designed as described in the results. The CAR cDNA was cloned into a pHIV.7 lentiviral vector. CD19-CAR was generated similarly, replacing BAFF-R antibody single-chain variable fragment (scFv) with CD19 antibody scFv that was derived from a clinically tested CD19-CAR (Qin et al., Science Translational Medicine, 11(511) 2019). Lentiviruses were produced in 293FT cells, concentrated, and titered with Jurkat cells. Tn/mem or subject T cells were isolated and activated with Human T-Activator CD3/CD28 beads (Life Technologies) for 24 hours, followed by transduction with lentivirus encoding CAR at a multiplicity of infection (MOI)=1. Protamine sulfate was used as a transduction enhancer for lentivirus transduction to generate CAR-T cells. The optimal MOI was determined after testing the MOI range from 0.1 to 10 to maximize the CAR-T cell potency while minimizing residual lentivirus effect (WPRE and VSVG) in the CAR-T cells. The CAR-T cells were further activated with CD3/CD28 bead stimulation for seven days after which the beads are removed, and the CAR-T cells were expanded for an additional seven days. Non-CAR-T cells were non-transduced T cells from the same donor, expanded following the CAR-T cell protocol, and used as a control.

### In Vitro Functional Assays

### Degranulation Assay

CAR-T cells were incubated with target cells at an effector-to-target (E:T) ratio of 2:1 in complete RPMI 1640 medium containing GolgiStop™ Protein Transport Inhibitor Reagent (BD Bioscience) and CD107a APC antibody (BD Biosciences) for 6 hours. The cells were subsequently stained with anti-CD3 BV605 (BD Biosciences), anti-CD4 PE-Cy7 (BD Biosciences), anti-CD8 APC-Cy7 (BD Biosciences), and anti-EGFR BV421(BD Biosciences). Samples were evaluated on the Attune flow cytometer (Thermo Fisher Scientific) or the Fortessa flow cytometer (BD Biosciences); data were analyzed using FlowJo™ Version 10 software. Non-CAR T cells from the same patient were used as negative controls.

### Granule Release Assay

CAR-T cells and target cells were co-incubated for 72 hours at an E:T ratio of 4:1; the supernatant was collected after 72 hours; and analytes were quantified using a customized U-PLEX Human ELISA kit from Meso Scale Diagnostics, following the manufacturer's instructions (Rockville, MD, USA).

### Direct Killing Assay

To evaluate the cytolytic function of CAR-T cells on tumor cells, CAR-T cells were co-incubated with GFP-positive target cells at an E:T ratio of 20:1 for 24 hours. Live dye staining with Sytox Blue (Thermo Fisher) identified the percentage of live GFP-positive tumor cells; the samples were run on the Attune flow cytometer.

### In Vivo Modeling

NOD scid gamma (NSG) mouse breeding pairs were purchased from The Jackson Laboratory (stock no. 005557) to establish a breeding colony. Mice (8-12 weeks old) received an intravenous (IV) challenge with a luciferase-expressing human tumor cell line (optimized in a separate experiment), randomized into test groups (5 mice per group), and treated with a single IV treatment dose, typically with non-CAR-T cell group receiving 10×10⁶ cells total T cells and CAR-T cell groups receiving 2×10⁶ cells CAR-T cells out of 10×10⁶ total T cells. The tumor burden was quantified weekly by bioluminescent signal intensity on isoflurane-anesthetized mice that received a subcutaneous injection of D-luciferin (150 μg luciferin/1 g mouse body weight) 10 minutes prior to IVISR imaging (PerkinElmer, Waltham, MA). Survival data were presented and reported in Kaplan-Meier plots.

### Statistical Analysis

All statistical analyses were performed with GraphPad Prism software (San Diego, CA). Data are reported as means±SD and analyzed by a student's t test. Unpaired t test comparisons were performed with granule protein release data, and a log rank test was performed for the animal studies. Typical comparisons were between non-CAR T cells and antigen-specific CAR-T cells, with the following convention: * for p<0.05; ** for p<0.01; *** for p<0.001.

### Results

### Generation of Novel Anti-BAFF-R MC10029 CAR

The MD Anderson Cancer Center Monoclonal Antibody Core Facility was contracted to generate an anti-BAFFR monoclonal antibody (mAb) using BAFF-R expressing NIH/3T3 cells as the immunogen. (FIG. 1A) Screening of candidate hybridoma clones against BAFF-R expressing 293FT cells was performed for identifying the lead antibody-producing clone. (FIG. 1B) Remarkably, hybridoma clone 21 (C21) supernatants exhibited dose-dependent and antigen-specific binding patterns. (FIGS. 1C and 1D) This antigen-specific binding was further confirmed on the antibody purified from C21 hybridoma supernatant. (FIG. 1E) Following confirmation of the lead hybridoma clone, the cDNA sequences of the heavy and light chain variable regions of the C21 mAb were identified for constructing the MC10029 CAR.

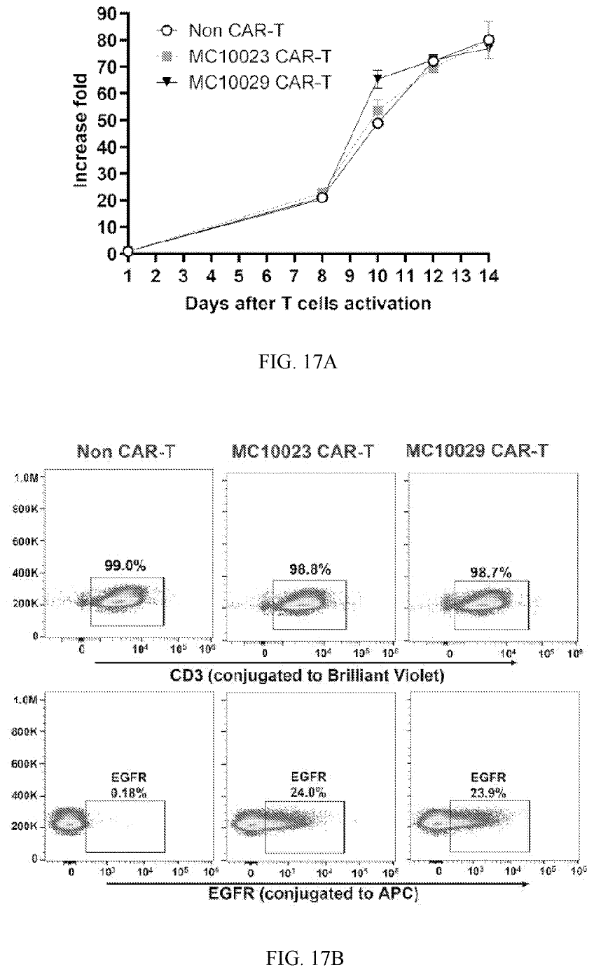

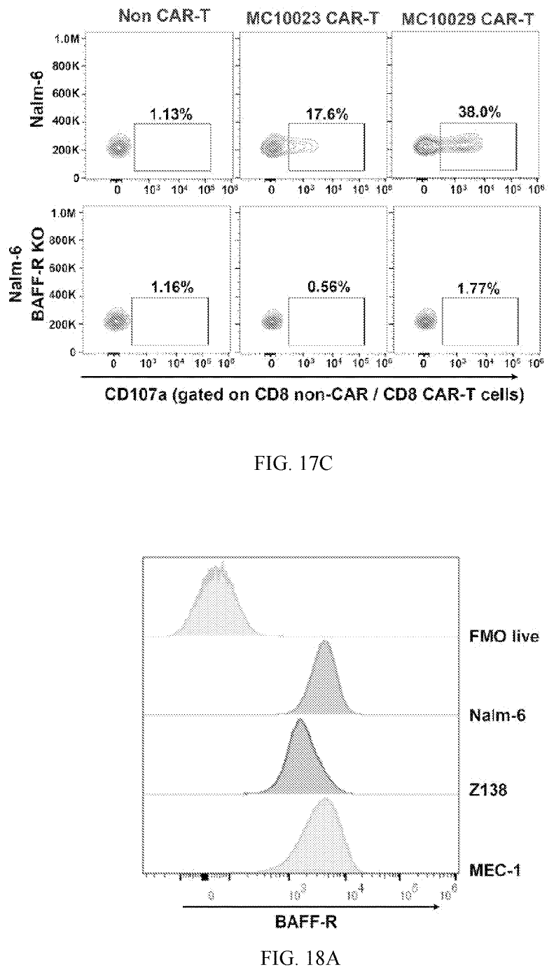

A second generation BAFF-R CAR (MC10029 CAR) was generated using a clinically approved lentiviral vector (Dong et al., Cancer Immunology, Immunotherapy, 69(10), 2139-2145, 2020). The construct includes the scFv of our new BAFF-R antibody with the following elements in tandem: IgG4 hinge, CD28 transmembrane domain, CD28 costimulatory domain, CD3ζ, and tEGFR (FIG. 2A). Selection of the CD28 costimulatory domain was empirically determined by comparing the antigen-specific cytotoxicity of BAFF-R CAR constructs containing either CD28 or 4-1 BB (FIGS. 17A-17C). Research grade MC10029 CAR-T cells were reproducibly generated with the specific requirements of cell quality, CAR-T cell specific characterization, and CAR-T cell fold expansion (FIGS. 2B-2E) to ensure the quality of the experimental CAR-T cells.

### MC10029 CAR-T Cells Exhibit Antigen-Specific Cytotoxicity Against Acute Lymphocytic Leukemia (ALL) in Both In Vitro and In Vivo Models

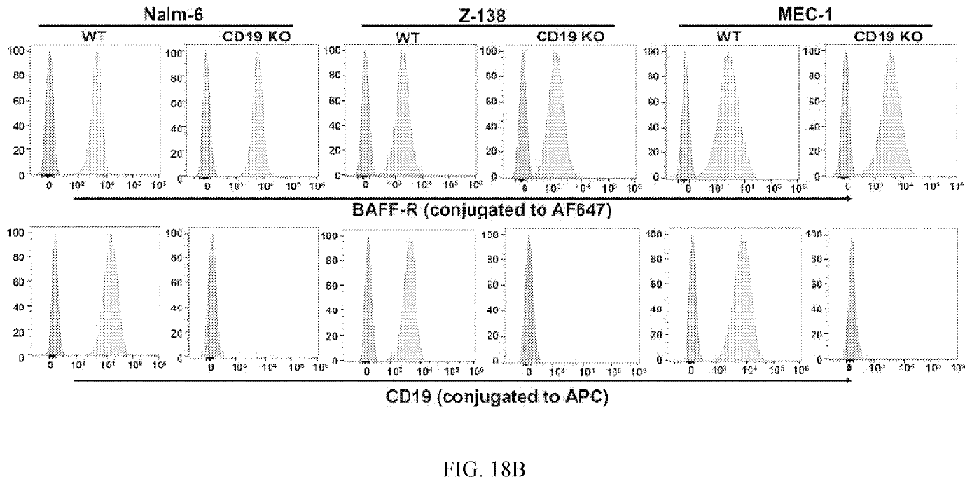

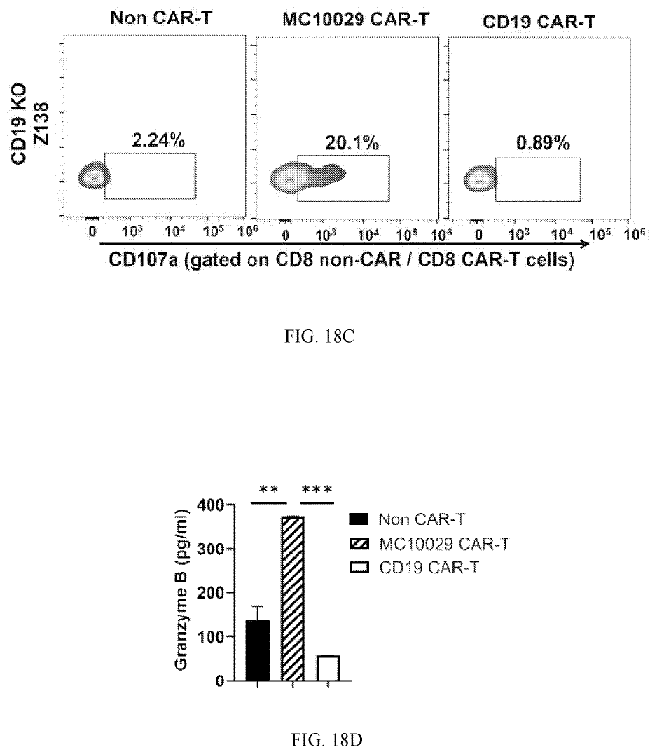

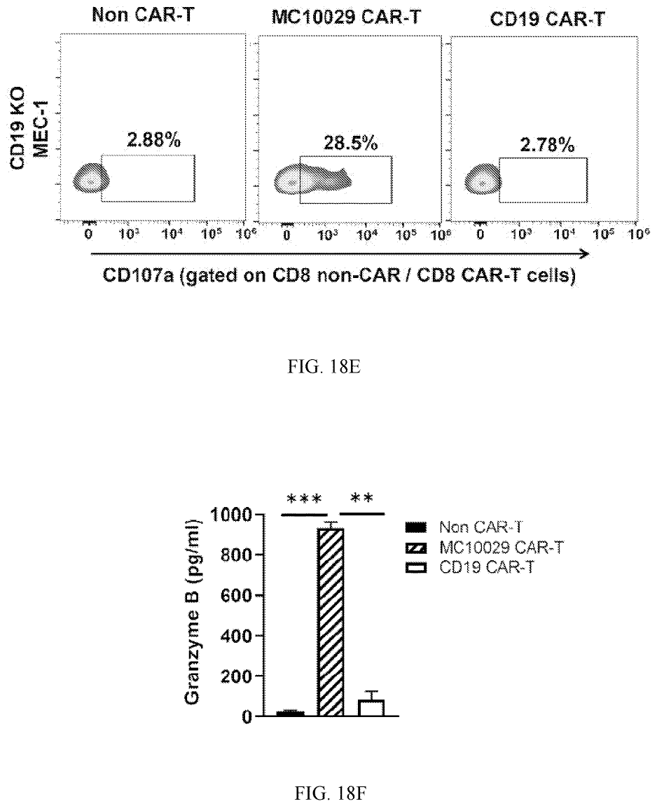

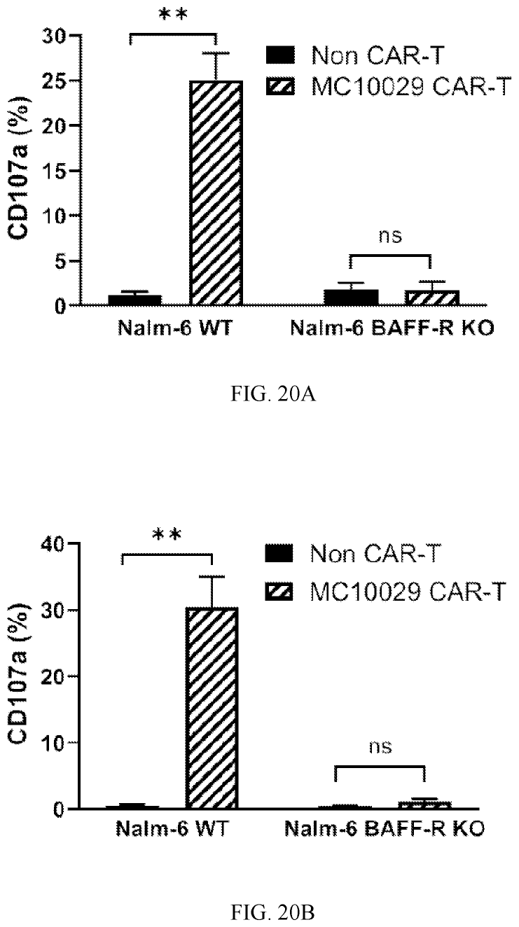

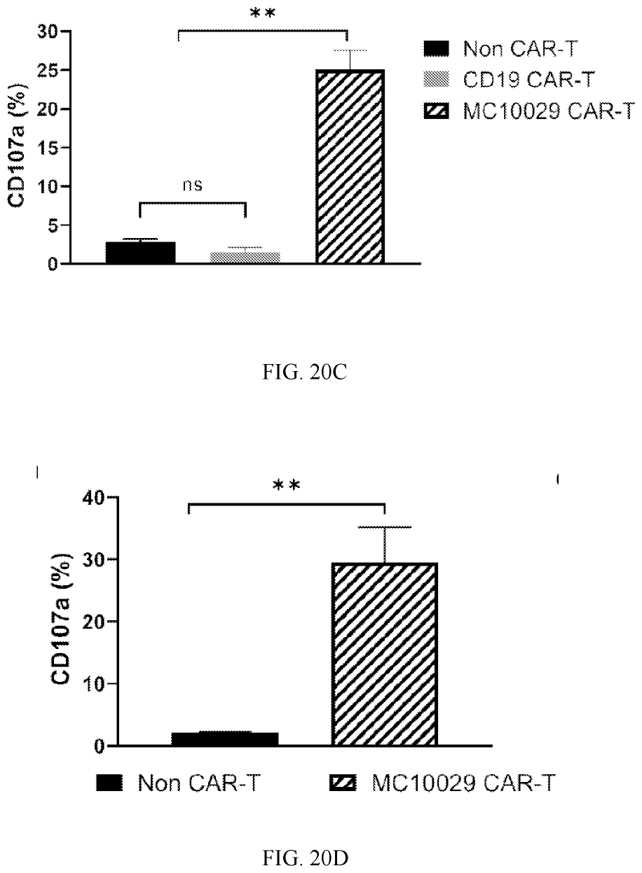

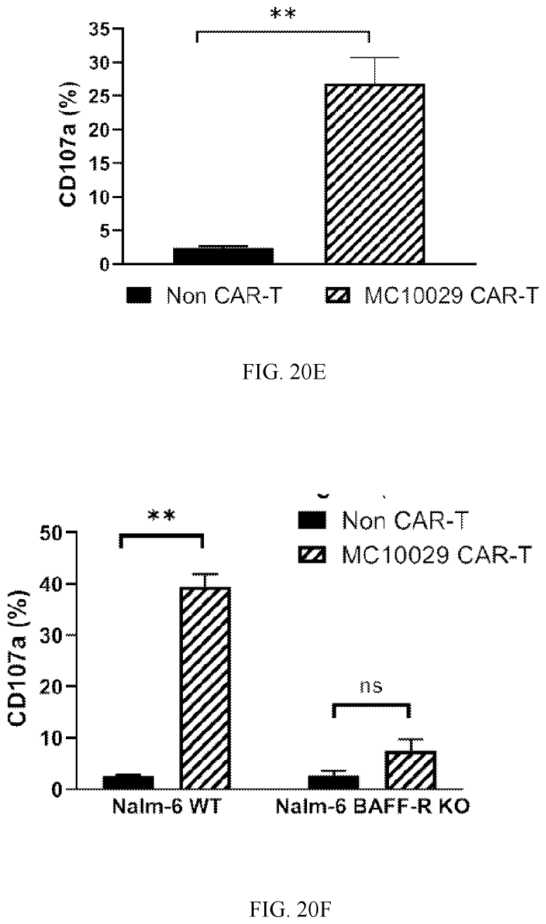

BAFF-R expression was confirmed in the cell lines used to characterize MC10029 CAR-T cell function (FIG. 18A). After confirming BAFF-R expression on Nalm-6, an ALL cell line, the antigen-specific cytotoxic properties of MC10029 CAR-T cells against wild-type (WT) Nalm-6 but not BAFF-R KO Nalm-6 cells were shown (FIG. 1A, FIG. 20A; FIG. 4B, and FIG. 20B). The CD107a degranulation assay was either gated on CD4 CAR-T cell populations. Granzyme B release was only observed when MC10029 CAR-T cells were incubated with WT Nalm-6 (FIG. 4C). Cytolysis, as determined with the loss of target cells engineered to express green fluorescent protein (GFP), was observed when MC10029 CAR-T cells were incubated with the GFP-labeled, WT Nalm-6, but not BAFF-R KO Nalm-6 cells, again confirming antigen-specific cytotoxicity (FIG. 4D).

The therapeutic efficacy of MC10029 CAR-T cells was assessed in NSG mice that were challenged with Nalm-6 tumor cells. Tumor changes were temporally monitored by bioluminescence imaging (FIG. 5A), and long-term survival was monitored to generate a Kaplan-Meier plot (FIG. 5B). Tumor growth and death were noted in the PBS treated mice within 42 days; the non-CAR-T cell treated group also showed advanced tumor progression. The MC10029 CAR-T cell treated mice displayed a considerable decrease in Nalm-6 tumor presence and a statistically significant survival rate to 120 days.

### MC10029 CAR-T Cells Remained Effective in Models of CD19 Antigen Loss

A Nalm-6-based model deficient in CD19 was generated to mimic antigen-escape disease. It was shown that MC10029 CAR-T cells retain antigen-specific cytotoxic function against the CD19 KO Nalm-6 cell line, while only background activity was observed for non-CAR-T cells and CD19 CAR-T cells (FIG. 6A and FIG. 20C). The robust MC10029 CAR-T cell activity against CD19-deficient tumor cells was confirmed by measuring granzyme B (FIG. 6B). The antitumor activity of MC10029 CAR-T cells against CD19-deficient tumors was consistently replicated in two additional CD19-deficient B-cell tumor models, namely CD19 KO Z-138 and CD19 KO MEC-1 (FIGS. 18B-18F). This confirmation further supports the initial observation made in the CD19 KO Nalm-6 model. NSG mice that were challenged with CD19 KO Nalm-6 tumor cells followed by one of four therapies: PBS, Non-CAR-T cells, MC10029 CAR-T cells, or CD19 CAR-T cells were next examined. Bioluminescence imaging showed tumor progression (FIG. 6C), and a Kaplan-Meier curve plotted survival (FIG. 6D). The control groups of PBS and Non-CAR-T cells were euthanized around 60 days due to excessive tumor burden; 80% of the mice in the CD19 CAR-T cell treatment group showed similar tumor burden as the controls with one mouse surviving to the termination of the 84-day experiment. The MC10029 CAR-T cell treatment group displayed a considerable decrease in tumor presence and a statistically significant survival.

### MC10029 CAR-T Cells Show Antigen-Specific Cytotoxicity of Against Lymphoma in Both In Vitro and In Vivo Models

Z-138 is a non-Hodgkin lymphoma (NHL) cell line that expresses BAFF-R (FIG. 18B). Antigen-specific cytotoxicity of MC10029 CAR-T cells against Z-138 was confirmed by using a CD107a degranulation assay (FIG. 7A and FIG. 20D) and by measuring granzyme B release (FIG. 7B). The therapeutic efficacy of MC10029 CAR-T cells was assessed in NSG mice that were injected with Z-138 tumor cells followed by one of three treatments. Bioluminescence imaging of the mice showed tumor progression (FIG. 3c), and survival was plotted (FIG. 7D). Excessive tumor burden led to the euthanasia of the control groups (PBS and Non-CAR-T cells) within 56 days. The MC10029 CAR-T cell treatment group showed a considerable decrease in tumor presence and a statistically significant survival rate.

### MC10029 CAR-T Cells Offer a Promising Option for Chronic Lymphocytic Leukemia (CLL)

CLL stands out as a significant unmet need in the realm of CAR-T cell therapy for B-cell hematological malignancies. Although CD19 CAR-T cell therapies have been pursued for CLL patients, complete remissions have not matched those reported for ALL or diseases like FL and MCL (Wang et al., N. Engl. J. Med., 382:1331-1342 (2020); Jacobson et al., Lancet Oncol., 23:91-103 (2022); Fowler et al., Nat. Med., 28:325-332 (2022); and Fraietta et al., Nat. Med., 24:563-571 (2018)). Considering the high expression of BAFF-R in CLL, the potential of BAFF-R-targeting MC10029 CAR-T therapy as an alternative option for CLL patients was explored. MEC-1, a CLL cell line, was first utilized to investigate the cytotoxicity of MC10029 CAR-T cells against CLL. The effectiveness of MC10029 CAR-T cells against MEC-1 cells was confirmed through granule degranulation, as shown in FIG. 8B and FIG. 20E. Furthermore, the release of granzyme B in response to the tumor cells provides additional evidence supporting the efficacy of MC10029 CAR-T cells in targeting and inducing cytotoxic effects on MEC-1 cells (FIG. 8B).

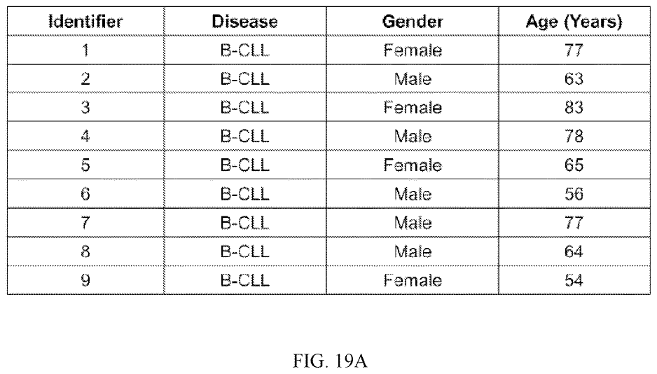

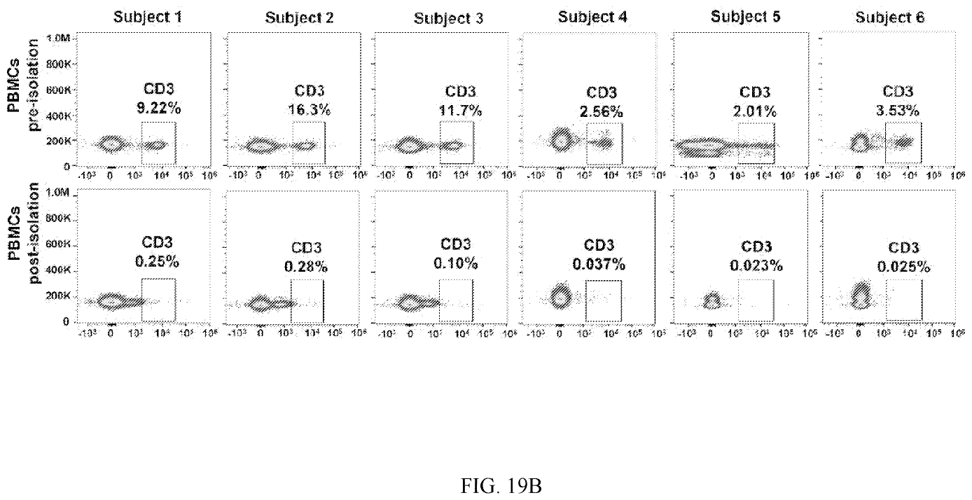

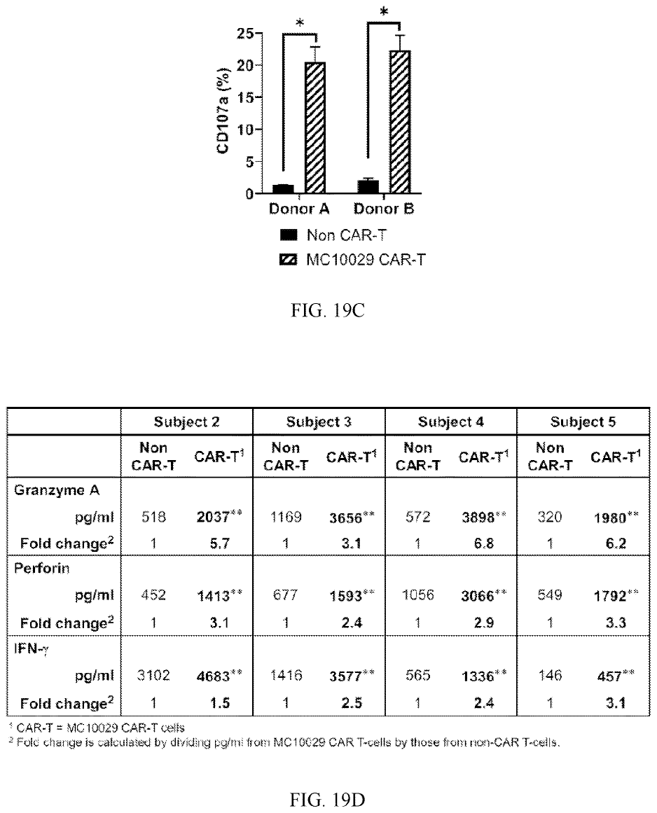

MC10029 CAR-T cells against primary B-cells isolated from subjects with CLL were next assessed. The selected subjects were three males and three females with an age range of 56 to 83 years. (FIG. 19A, Identifiers 1-6) B-cells were enriched from PBMCs of each subject, and BAFF-R expression was confirmed (FIG. 9A); B-cell enrichment effectively removed endogenous T cells (FIG. 19B). In the initial studies, two batches of MC10029 CAR-T cells and non-CAR-T cells were engineered from T cells isolated from two healthy donors. These batches were shown to be identical in viability, identity, and potency. (FIG. 11A-11C).

The activation of CD8+MC10029 CAR-T cells was comparable across the six primary CLL tumor cells following incubation (FIG. 9B, top panels and FIG. 19C). Furthermore, incubation of MC10029 CAR-T cells with CLL tumor cells resulted in the release of granzyme B (FIG. 9C). Four of the CLL subjects provided sufficient B-cells that could be evaluated for the release of additional granule proteins. Granzyme A, perforin, and IFN-γ were released in statistically significant amounts when the MC10029 CAR-T cells were incubated with primary CLL tumor cells (FIG. 19D).

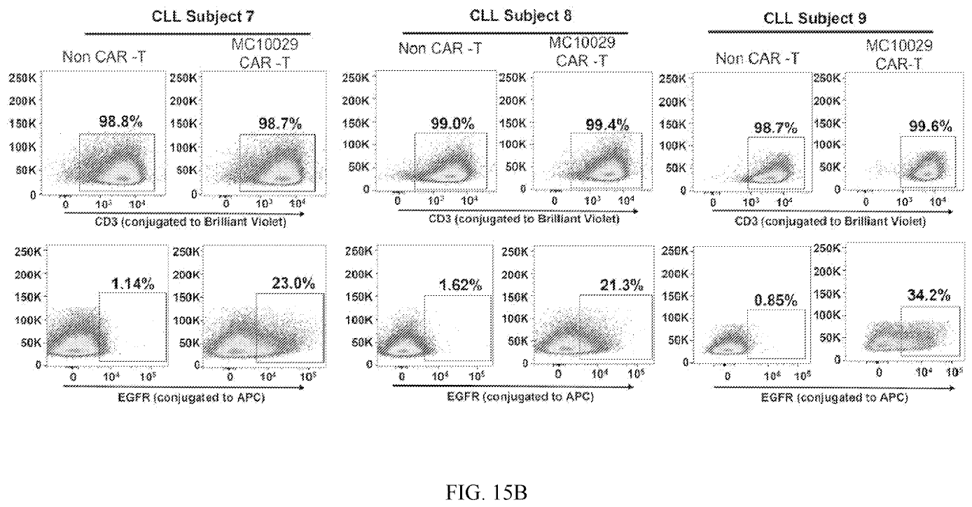

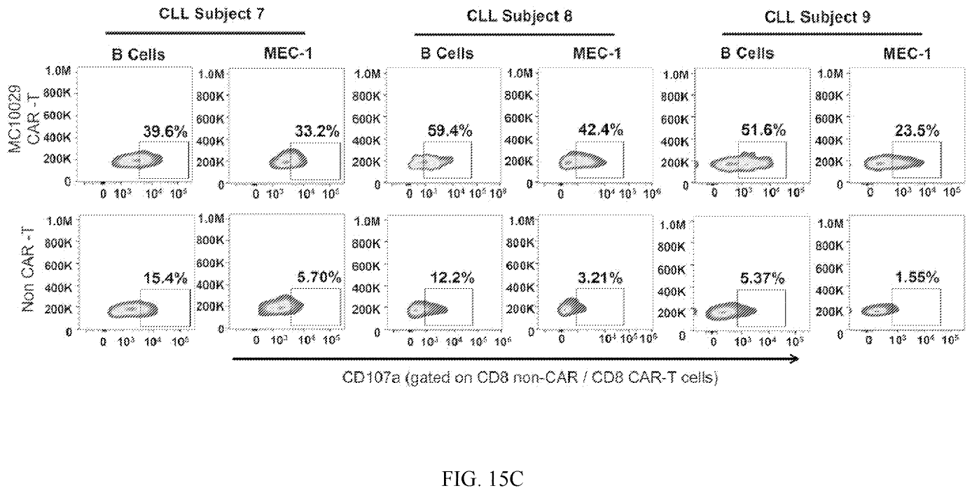

To model the efficacy of MC10029 CAR-T cells more realistically as a clinical therapeutic, three additional CLL subjects were identified (FIG. 19A, Identifier 7-9) to generate subject-derived MC10029 CAR-T cells for incubation with autologous B-cells. The MC10029 CAR-T cells that were engineered from three CLL subjects were characterized by monitoring fold expansion (>25; FIG. 15A), identity (>80% CD3 positive cells; FIG. 15B, top panels), and potency (>10% EGFR positive cells; FIG. 15B, bottom panels). These MC10029 CAR-T cells and their corresponding non-CAR-T cells (negative control) were then incubated with either the matching autologous B-cells or MEC-1 cells (positive control). The cytotoxicity of the subject-derived MC10029 CAR-T cells against autologous tumor cells was confirmed with a CD107a degranulation assay on CD8 (FIG. 15C). Non-specific activity is consistently observed when subject-derived Non-CAR-T cells are incubated with target cells, especially autologous B-cells.

### The Readiness of Advancing the MC10029 CAR-T Cell Therapy to the Clinic

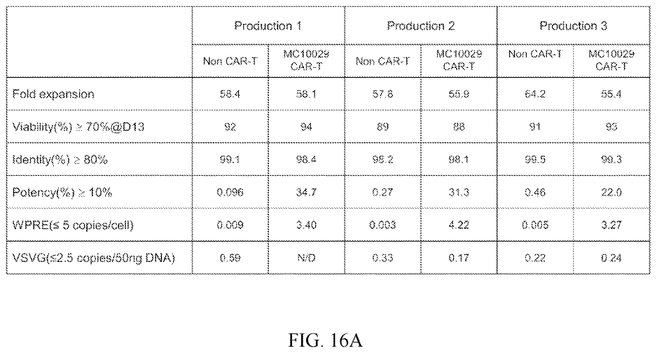

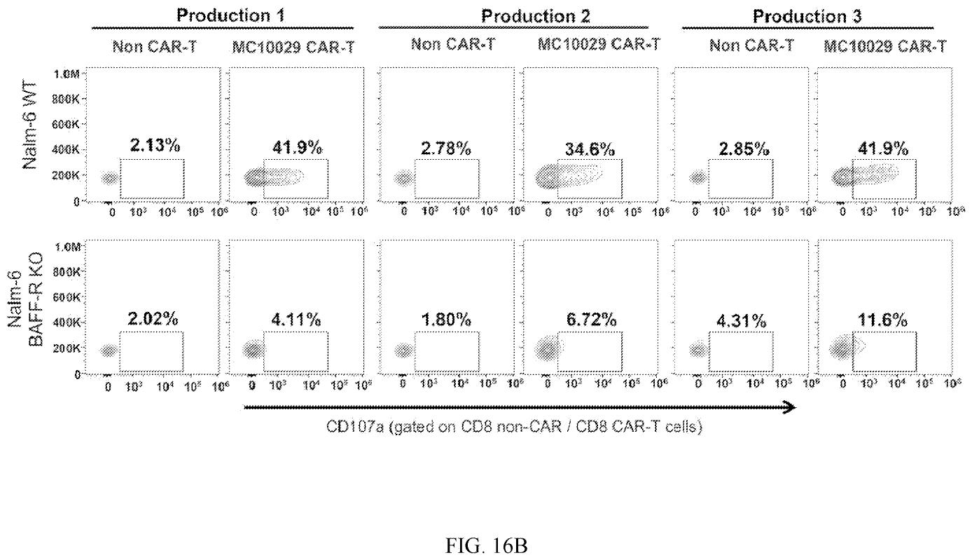

As part of an IND filing and in preparation for a Phase 1a/1b clinical trial, engineering runs were performed to yield three production batches of CAR-T cells using a MC10029-expressing lentiviral vector that was produced under GMP conditions. A collection of assays that will be used as the process quality control (QC) and final product QC assays have been established and validated. These assays were performed on the laboratory generated production batches (FIGS. 16A and 16B). The production batches of MC10029 CAR-T cells were evaluated for cell quality with Fold Expansion and Viability (>70%, as determined by Muse Cell Analyzer); CAR-T cell specific characterization with Identity (>70%, as determined by flow cytometry for CD3 positive cells) and Potency (>10%, as determined by flow cytometry for EGFR positive T cells); and Safety from adventitious viral agents by determining lentiviral copy number by two methods (<5 copies of Woodchuck Hepatitis Virus Posttranscriptional Regulatory Element (WPRE) per cell and <5 copies/50 ng DNA of vesicular stomatitis virus G glycoprotein (VSVG; encodes envelope gene sequences) as detected via a real-time qPCR assay). All three production batches of MC10029 CAR-T cells met the required criteria to be qualified as a product (FIG. 16A). Furthermore, antigen-specific cytotoxicity of these MC10029 CAR-T cells was confirmed using our standard degranulation assay (FIG. 16B, FIG. 20F). The successful qualification of these batches confirms their suitability for further clinical development.

Together, these results demonstrate that binders having two sets of three CDRs described herein (e.g., SEQ ID NOs:1-3 and 9-11) can bind to a BAFF-R polypeptide. Also as demonstrated herein, these binders can be engineered into molecules (e.g., antibodies, antigen binding fragments, antibody domains, CARs, cell engagers (e.g., BiTEs), or ADCs (e.g., antibody-radioconjugates)) that can be used to a treat a mammal (e.g., a human) having cancer (e.g., a B-cell cancer).

### Example 18: BAFF-R CAR-T Therapy for Sensitized Kidney Transplant Patients

Organ transplant demands, especially among patients with End Stage Kidney Disease (ESKD), far surpass the current availability of organs. This challenging situation is further compounded by the presence of pre-transplant high levels of anti-HLA antibodies, clinically known as sensitization (Heidt et al., Expert Rev. Clin. Immunol., 14:673-679 (2018)), making the matching process a highly complex one. Consequently, the waiting time on a transplant waitlist can be substantially lengthy, resulting in dire consequences where patients may tragically succumb to organ failure before receiving a life-saving organ transplant (Sapir-Pichhadze et al., J. Am. Soc. Nephrol., 27:570-578 (2016)).

Anti-HLA donor specific antibodies have been extensively documented for their critical role in kidney transplant rejection and resulting adverse outcomes. Several approaches have been employed to desensitize these patients; however, none of these explored therapeutic approaches has exhibited enduring clinical benefits.

This Example describes the use of MC10029 CAR T-cells to target B cells in kidney transplant recipients, thus desensitizing the patients and minimizing the risk of organ transplant rejection.

### Materials and Methods

### Cell Lines

The cell line of Nalm-6 was purchased from Deutsche Sammlung von Mikroorganismen und Zellkulturen GmbH (DSMZ, Germany), and the BAFF-R knockout variant (BAFF-R KO Nalm-6) was generated as described in Qin et al., Science Translational Medicine, 11(511) 2019. 293FT and Jurkat cells were obtained from the ATCC. Prior to cryopreservation, the cell lines underwent authentication for the desired antigen using flow cytometry.

### Human Blood Samples

Peripheral blood mononuclear cells (PBMCs) from healthy volunteer donors were isolated via leukapheresis using Leukocyte Reduction System (LRS) cones, by the Division of Transfusion Medicine, Mayo Clinic, Rochester, Minnesota, according to current regulatory requirements and described in Dietz et al., Transfusion 46:2083-2089 (2006).

The patients' blood procurement was performed part of a biorepository protocol. All patients provided written informed consent. Approximately 40 mL of blood were collected from consenting patients with two draws that were at least 2 weeks apart. Disease-characteristics of these patients were recorded.

PBMCs Isolation from Patients' Blood Samples

The collected peripheral blood from subjects was diluted 1:1 v/v with PBS and overlaid the above diluted blood sample (20 mL) on the top of 10 mL Ficoll (Sigma). The gradient was centrifuged for 20 minutes with 2000 rpm at room temperature. Subsequently, the PBMC layer was carefully separated and isolated. To assess the number and viability of PBMCs, a cell counter (TC 20 Automated Cell Counter, Bio-Rad) was utilized.

### Generation of Patient-Derived MC10029 CAR-T Cells

All patients were consented for two blood draws, typically spaced apart by two weeks, each involving a collection of 12-20 mL of blood. The first blood draw was used for the generation of patient-derived CAR T-cells. The harvested PBMCs were subjected to the isolation of T cells using the Pan T cell isolation kit (Miltenyi Biotec, Germany). In brief, the PBMCs were initially labeled with Biotin-Antibody cocktail followed by MicroBead Cocktail in FACS buffer. The labeled cells were then applied onto a LD column for magnetic cell separation. The unlabeled T cells that passed through the column were collected for CAR T-cell production.

A second-generation BAFF-R-CAR(MC10029) was generated consisting of a novel BAFF-R antibody scFv, IgG4 transmembrane, CD28, and CD3ζ intracellular signaling domains. In addition, the CAR construct was designed to include a safety switch, tEGFR (truncated human epithelial growth factor receptor), enabling the depletion of CAR T-cells after dosing with an FDA-approved anti-EGFR mAb, cetuximab. The CAR cDNA was cloned into pHIV.7 lentiviral vector. Lentiviruses were produced in 293FT cells, concentrated, and titered with Jurkat cells. T isolated from patients' PBMCs were divided into two aliquots. One aliquot advanced to be expanded as non-transduced (non-CAR) T cells used as an alloreactivity control, whereas the remaining cells advanced to CAR T-cell production. To generate patient-derived CAR T-cells, T cells were activated with Human T-Activator CD3/CD28 beads (Life Technologies) for 24 hours followed by transduction with CAR lentivirus at multiplicity of infection (MOI=1); the CAR T-cells were further activated with CD3/CD28 bead stimulation for six days after which the beads are removed, and the CAR T-cells are permitted to expand for an additional seven days. Non-CAR T-cells are non-transduced T cells from the same donor, expanded following the CAR T-cell protocol. Each batch of CAR T-cells were evaluated for cell quality with Fold Expansion and Viability (as determined by Trypan Blue staining) and CAR T-cell specific characterization with Identity (CD3 positive cells) and Potency (EGFR positive T cells) by flow cytometry. The characterized patient-derived MC10029 CAR T-cells were cryopreserved for subsequent functional assays.

### Isolation of Autologous B Cells for CAR T-Cell Functional Assays

When patients were available for the second blood draw, autologous B cells were isolated from PBMCs using the EasySep™ Human B cell isolation kit (STEMCELL technologies, Canada). Briefly, the Isolation Cocktail was added to the PBMCs, followed by the addition of RapidSpheres™ for incubation. Subsequently, the tube was placed into the magnet for negative selection. The isolated B cells were stained with anti-CD3 BV605 (BD Biosciences), anti-CD20 BUV395 (BD Biosciences), and anti-BAFF-R-AF647(BD Biosciences) for characterization. These freshly isolated autologous B cells served as the target cells to stimulate cryopreserved MC10029 CAR T-cells in T-cell functional assays. A minimum of two million enriched B cells was necessary for both degranulation and granule release assays. Out of the 10 enrolled patients, we successfully collected between 1.5-2.65 million B cells from each of the 7 patients (6 kidney transplant patients plus the lung transplant patient), which allowed for at least one CAR T-cell functional assay to be conducted. For the remaining three kidney transplant patients (Patients 7-9), challenges were encountered in obtaining sufficient autologous B cells (yields less than 0.5 million cells). Consequently, their cryopreserved CAR T-cells were tested for the antigen-specific functionality using target cell lines (Nalm-6 and BAFF-R KO Nalm-6).

### Degranulation Assays

CAR T-cells were incubated with target cells at an effector-to-target (E:T) ratio of 2:1 in complete RPMI 1640 medium containing GolgiStop Protein Transport Inhibitor Reagent (BD Bioscience) and CD107a APC antibody (BD Biosciences) for 6 hours. The cells were subsequently stained with anti-CD3 BV605 (BD Biosciences), anti-CD4 PE-Cy7 (BD Biosciences), anti-CD8 APC-Cy7 (BD Biosciences) and anti-EGFR BV421(BD Biosciences). Samples were run on Attune flow cytometer (Thermo Fisher Scientific) or Fortessa flow cytometer (BD Biosciences) and analyzed using FlowJo™ Version 10 software. Non-CAR T-cells from the same patient were used as negative controls.

### Granule Release Assay

CAR T-cells and target cells were co-incubated for 72 hours at an E:T ratio of 4:1. After the incubation period, the supernatant was collected and evaluated for granule release. The levels of granule proteins involved in cytotoxic activity, such as granzyme B, granzyme A and perforin, were quantified using a customized U-PLEX Human ELISA kit from Meso Scale Diagnostics, following the manufacturer's instructions (Rockville, MD, USA). This multiplex kit enabled the simultaneous measurement of multiple secretory proteins associated with CAR T-cell function.

### Direct Killing Assay Using B Cells as Target Cells

The direct killing assay using B cells as the target cells was established with MC10029 CAR T-cells derived from healthy donors. This established assay was then applied to patient-derived MC10029 CAR T-cells. MC10029 CAR T-cells were co-incubated with B cells at an E:T ratio of 10:1 for 1 hour. The cells were subsequently stained with anti-CD20 BUV395 (BD Biosciences) and Sytox Blue (Thermo Fisher). The samples were evaluated using a Fortessa flow cytometer (BD Biosciences), and the resultant data were analyzed using FlowJo™ Version 10 software. Non-CAR T-cells from the same donors were served as negative controls.

### Statistical Analysis

All statistical analyses were performed with the GraphPad Prism software. Data are reported as means±SEM and analyzed by a student's t test.

### Results

### Study Design

The primary objective of this study was to investigate the cytotoxicity of patient-derived MC10029 CAR T-cells against their own autologous B cells. Inclusion criteria consisted of adult patients aged 18 and above, exhibiting a high sensitization level with a calculated panel reactive antibody (cPRA) of 98% or higher, and expressing their willingness to provide informed consent, clinical data, and blood samples. Patients who had received desensitization therapy were excluded from the study. A total of 10 eligible patients participated in this research, consisting of nine kidney transplant patients and one patient who had received lung transplant. The patient and disease-related information is detailed in Table 2 and Table 3.

TABLE 2
Patient information, characterization of MC10029 CAR T-cells, and their functional responses to autologous B cells.
Patient information

| | Patient 1 | Patient 2 | Patient 3 | Patient 4 | Patient 5 | Patient 6 |
|---|---|---|---|---|---|---|
| Age (years) | 63 | 70 | 52 | 25 | 51 | 34 |
| Gender | Male | Female | Female | Female | Female | Male |
| Race | African | Caucasian | African American | Caucasian | African American | Caucasian |
| | American | | | | | |
| Disease | Diabetic | P-ANCA | FSGS/allograft | FSGS/allograft | FSGS/allograft | Microscopic |
| | Nephropathy/ | vasculitis | failure | failure | failure | polyangiitis/ |
| | allograft | | | | | allograft failure |
| | failure | | | | | |
| Previous | Yes | No | Yes | Yes | Yes | Yes (twice) |
| Organ | | | | | | |
| Transplant | | | | | | |

Production of patient-derived MC10029 CAR T-cells

| | Non | | Non | | Non | | Non | | Non | | Non | |
|---|---|---|---|---|---|---|---|---|---|---|---|---|
| | CAR-T | CAR-T | CAR-T | CAR-T | CAR-T | CAR-T | CAR-T | CAR-T | CAR-T | CAR-T | CAR-T | CAR-T |
| Fold | 90.3 | 112 | 81.6 | 74.7 | 58 | 70 | 100 | 98 | 121 | 145 | 90 | 89 |
| Expansion ^{b} | | | | | | | | | | | | |
| Viability (%) ^{c} | 85 | 83 | 94.3 | 95 | 92.9 | 94.0 | 97.0 | 96.0 | 94.5 | 90.7 | 92.8 | 93.7 |
| Identity (%) ^{d} | 99.7 | 99.5 | 96.6 | 97.5 | 99.7 | 99.7 | 97.8 | 98.2 | 99.1 | 99.2 | 99.3 | 99.4 |
| Potency (%) ^{e} | 0.71 | 28.1 | 0.57 | 25.1 | 0.91 | 28.2 | 0.46 | 27.9 | 0.79 | 28.5 | 0.83 | 20.3 |

Functional analysis of CAR T-cells

| | Non | | Non | | Non | | Non | | Non | | Non | |
|---|---|---|---|---|---|---|---|---|---|---|---|---|
| | CAR-T | CAR-T | CAR-T | CAR-T | CAR-T | CAR-T | CAR-T | CAR-T | CAR-T | CAR-T | CAR-T | CAR-T |
| Degranulation ^{f} | 3.4 | 33.3 | ND | 51 | 3.83 | 31.1 | 4.53 | 13.1 | ND | 14.9 | ND | 49.8 |
| Granzyme | 583 | 3310** | 746 | 8052** | 403 | 2500** | 270 | 2313** | 3373 | 9252** | 299 | 2564** |
| A(pg/ml) | | | | | | | | | | | | |
| Fold change ^{g} | 1 | 5.7 | 1 | 10.8 | 1 | 6.2 | 1 | 8.6 | 1 | 2.7 | 1 | 8.6 |
| Granzyme | 385 | 1188** | 154 | 2587** | 89 | 447** | 606 | 1625** | 289 | 889** | 456 | 2655** |
| B(pg/ml) | | | | | | | | | | | | |
| Fold change ^{g} | 1 | 3.1 | 1 | 16.8 | 1 | 5.0 | 1 | 2.7 | 1 | 3.1 | 1 | 5.8 |
| Perforin | 1064 | 3690** | 716 | 3756** | 524 | 2234** | 399 | 1857** | 1788 | 4650* | 790 | 3063** |
| (pg/ml) | | | | | | | | | | | | |
| Fold change ^{g} | 1 | 3.5 | 1 | 5.3 | 1 | 4.3 | 1 | 4.7 | 1 | 2.6 | 1 | 3.9 |

P-ANCA, Perinuclear anti-neutrophil cytoplasmic antibodies; FSGS, Focal segmental glomerulosclerosis; CAR-T = MC10029 CAR T-cells; ND = Not determined; Insufficient PBMCs required preferred production of MC10029 CAR T-cells and did not allow for both degranulation and secreted protein determination via ELISA
^{a} Patient 2 received prior treatment with rituximab in 2020 and cyclophosphamide until 2022.
^{b} Fold expansion is a parameter that quantifies the increase in cell numbers during the 14-day CAR T-cell process, starting from the original 1 million cells.
^{c} Viability is measured by trypan blue with the acceptance criteria of ≥70% at day 14.
^{d} Identity is measured using a CD3 antibody with the acceptance criteria of ≥80% cells having the characteristic CD3 surface marker.
^{e} Potency is measured using an EGFR antibody with the acceptance criteria of ≥10% cells having the tEGFR surface marker, a proxy marker for the CAR T-cells.
^{f} Degranulation of T cells is measured with CD107a surface expression; CD107a % is determined by flow cytometry
^{g} Fold change is calculated by dividing the concentrations of granule protein released b from MC10029 CAR T-cells by those from non-CAR T-cells.
*/** The levels of granule proteins are presented as means from a total of 3 or 4 replicates, the P values were measured for the comparison between CAR-T and Non CAR-T (* P <0.05; ** P <0.01)

TABLE 3
Production of patient-derived MC10029 CAR T-cells from additional sensitized patients who experienced
organ transplant failure.

| | | | |
|---|---|---|---|
| Patient 7 | | | |
| (Outside from Mayo | | | |
| Clinic) | Patient 8 ^{a} | Patient 9 ^{a} | Patient 10 |

Patient information

| | | | | |
|---|---|---|---|---|
| Age (Years) | N/A | 51 | 29 | 68 |
| Gender | N/A | Female | Female | Female |
| Race | N/A | White | White | White |
| Disease | Kidney allograft failure | PKD/kidney allograft | Congenital renal | Idiopathic |
| | | failure | dysplasia/kidney | pulmonary |
| | | | allograft failure | fibrosis |
| Previous Organ | Yes | Yes | Yes | Yes |
| Transplant | | | | |

Production of patient-derived MC10029 CAR T-cells

| | Non CAR-T | CAR-T | Non CAR-T | CAR-T | Non CAR-T | CAR-T | Non CAR-T | CAR-T |
|---|---|---|---|---|---|---|---|---|
| Fold Expansion ^{b} | 66.7 | 68.8 | 150 | 151 | 150 | 134 | 113 | 115 |
| Viability (%) ^{c} | 86 | 82 | 94.1 | 95 | 94.9 | 94.5 | 97.1 | 96.4 |
| Identity (%) ^{d} | 97.7 | 98.7 | 99.3 | 99.1 | 99.3 | 99.3 | 95.8 | 95.1 |
| Potency (%) ^{e} | 0.22 | 33.7 | 0.34 | 34.0 | 0.62 | 18.1 | 1.10 | 47.1 |

N/A = Not available; CAR-T = MC10029 CAR T-cells; PKD = Polycystic kidney disease
^{a} Received prior treatment with rituximab in 2020 and cyclophosphamide until 2022
^{b} Fold expansion is a parameter that quantifies the increase in cell numbers during the 14-day CAR T-cell process, starting from the original 1 million cells.
^{c} Viability is measured by trypan blue with the acceptance criteria of ≥70%.
^{d} Identity is measured using a CD3 antibody with the acceptance criteria of ≥80% cells having the characteristic CD3 surface marker.
^{e} Potency is measured using an EGFR antibody with the acceptance criteria of ≥10% cells having the tEGFR surface marker, a proxy marker for the CAR T-cells

### Targeting BAFF-R on Patients' B Cells by MC100029 CAR T-Cells

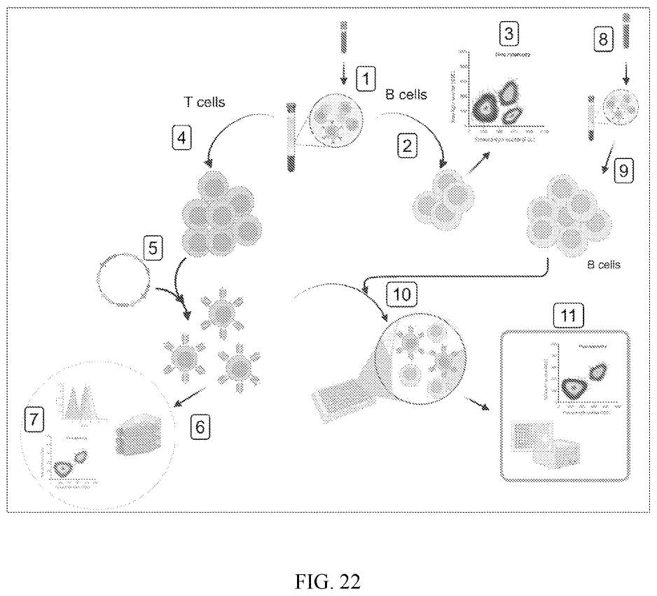

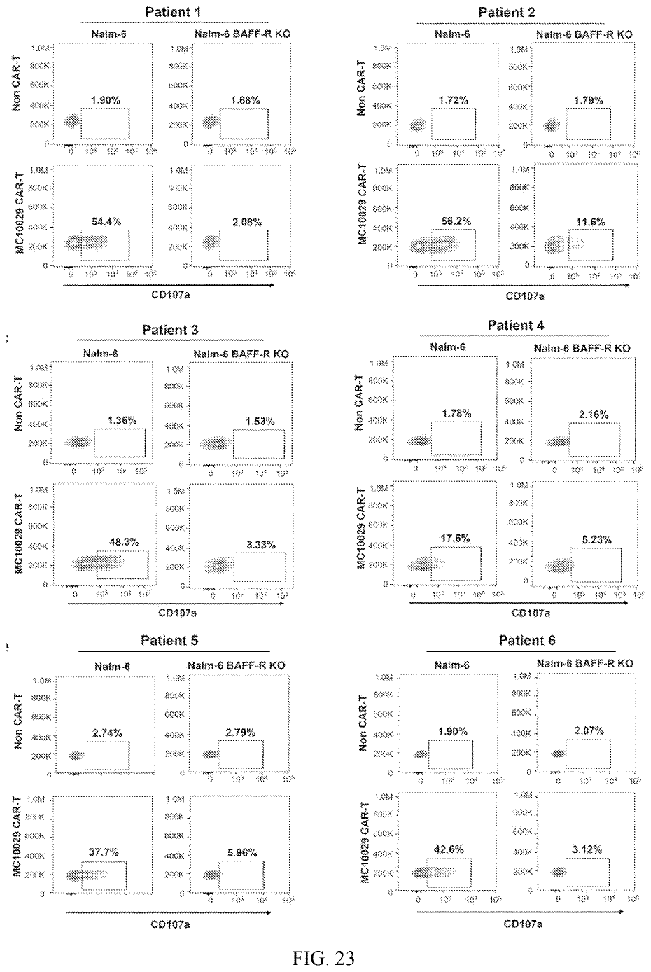

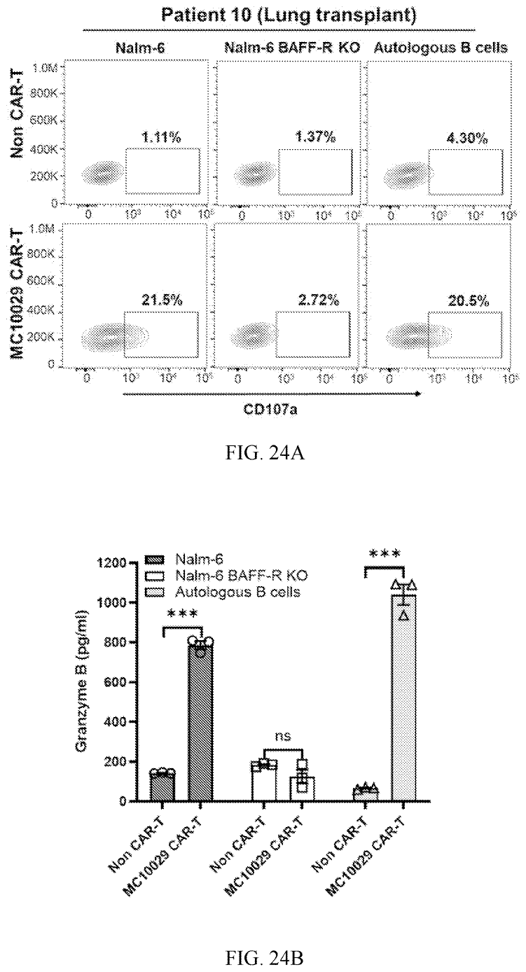

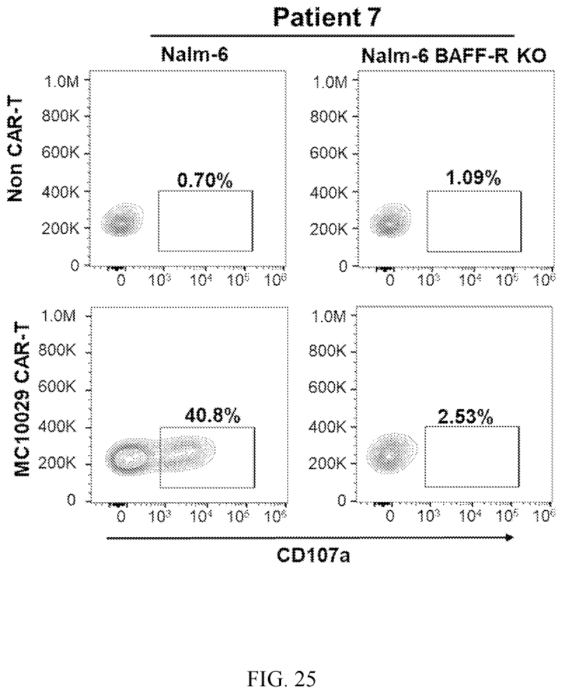

A protocol (FIG. 22) was developed to generate patient-derived CAR T-cells from limited blood samples (12-20 mL) and optimized to isolate enriched autologous B cells from the same patient that served as target cells in the presented CAR T-cell functional experiments (Table 2 and FIG. 23). CAR T-cells were successfully generated for all 10 enrolled patients (Table 2 and Table 3). Adequate amounts of autologous B cells were obtained from seven out of the 10 patients, including the lung transplant recipient, enabling the assessment of cytotoxicity of patient-derived CAR T-cells against autologous B cells (Table 2 and FIG. 24, respectively). For the remaining three patients from whom we could not isolate sufficient B cells for autologous experiments, the antigen-specific functionality of their CAR T-cells was validated using target cell lines (Nalm-6 and BAFF-R KO Nalm-6) (FIG. 25).

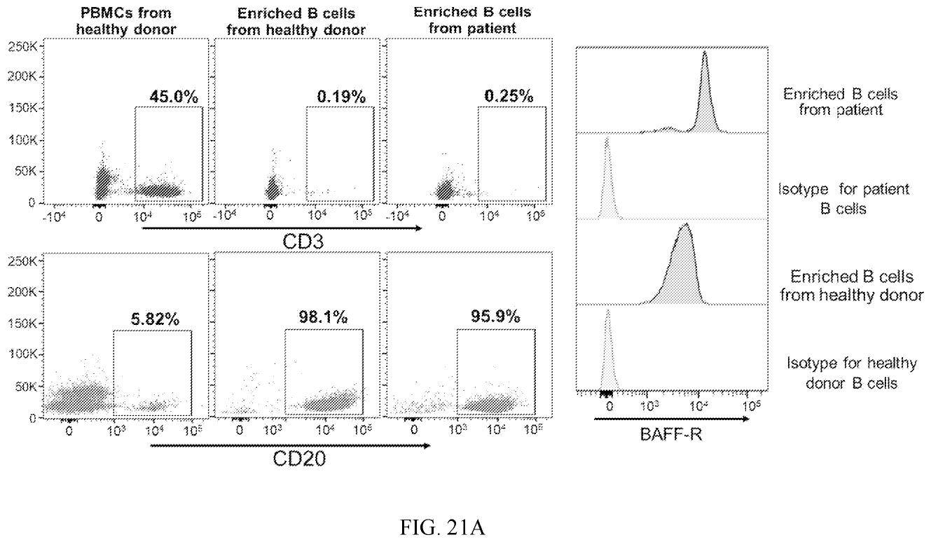

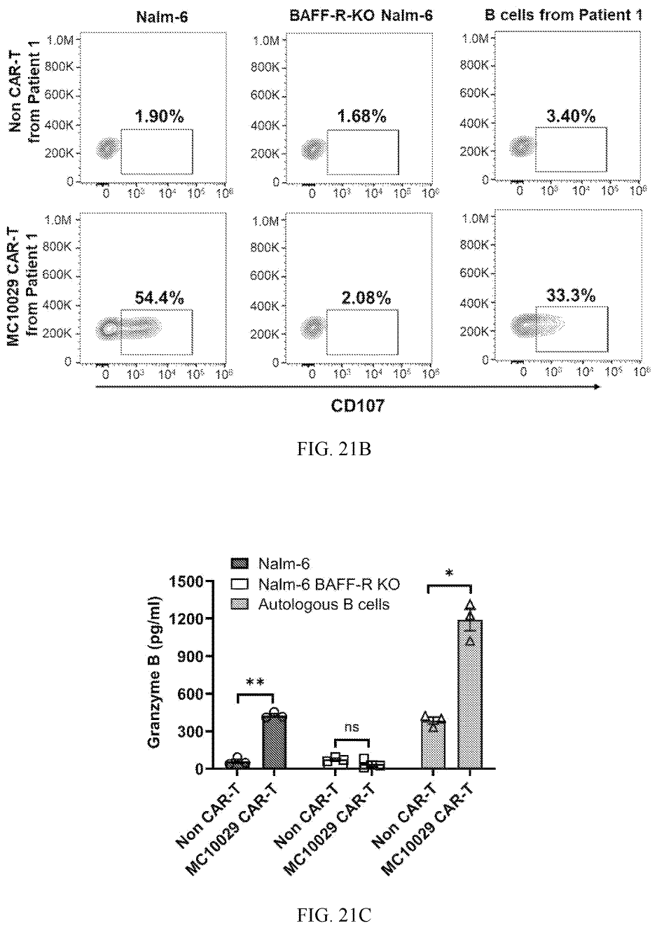

Autologous CD20-positive B cells from sensitized patients were enriched from blood samples and subsequently used as target cells. Enriched B cells obtained from a healthy donor were utilized as a control. This enrichment process efficiently depleted CD3-positive T cells to mitigate any non-specific background that these T cells might introduce in the following CAR T-cell functional assays (FIG. 21A). Notably, B cells from sensitized patients displayed BAFF-R expression levels comparable to those found in B cells from healthy donors (FIG. 21A), highlighting the potential of targeting BAFF-R for B cell depletion in desensitization strategies. The cytotoxicity of patient-derived MC10029 CAR T-cells against autologous B cells was assessed by evaluating their degranulation activity and further confirmed through the measurement of granzyme B release. FIG. 21 contains representative patient data (Patient 1 in Table 2), demonstrating the readily detectable degranulation activity of MC10029 CAR T-cells in response to autologous B cells (FIG. 21B). Furthermore, the CAR T-cells exhibited a significant release of cytotoxic granzyme B upon encountering autologous B cells (FIG. 21C). The non-CAR T-cells from the same patient served as a control in both degranulation assay and granzyme B release experiments, representing the baseline level of T cell activation observed in the patient. Importantly, the CAR T-cell functionality was shown to be antigen-specific, as evidenced by the activation of CAR T-cells exclusively against Nalm-6 cells expressing endogenous BAFF-R, while showing no activation against their genetically engineered variants lacking BAFF-R expression (FIGS. 21B-21C). These data validate the autologous experimental system and support utilizing MC10029 CAR T-cell therapy as a desensitization strategy.

### Evaluation of Patient-Derived MC10029 CAR T-Cells Against Autologous B Cells

Adequate amounts of autologous B cells were obtained from seven out of the 10 enrolled patients, which enabled the assessment of cytotoxicity of patient-derived CAR T-cells against autologous B cells. This included six kidney transplant recipients (Patients 1-6 in Table 2) and one lung transplant recipient (Patient 10 in FIG. 24). The functional analysis data of the six kidney transplant patients are presented in Table 2. The CAR T-cells derived from these patients consistently exhibited degranulation activities against their autologous B cells, as evidenced by the release of cytotoxic proteins (granzyme A, granzyme B, and perform). To account for variations in baseline level of T cell activation among patients, we calculated the fold change between MC10029 CAR T-cells and non-CAR T-cells, thereby normalizing the data for inter-patient comparisons (Table 2). Furthermore, the CD107a degranulation data also served as compelling evidence of the cytotoxicity of patient-specific CAR T-cells against autologous B cells. Consistently positive staining for CD107a on the surface of these CAR T-cells indicated robust degranulation activity triggered in response to autologous B cells (Table 2). Similar to observations in kidney transplant patients, MC10029 CAR T-cells derived from the lung transplant patient elicited cytotoxicity against autologous B cells, as substantiated by both degranulation and the release of granzyme B (FIG. 24). The functionality of patient-derived MC10029 CAR T-cells was confirmed to be antigen-specific, exhibiting T-cell degranulation upon interaction with BAFF-R-positive Nalm-6 cells. Conversely, no reaction was observed when these CAR T-cells interacted with a variant Nalm-6 cell line that was genetically modified to lack BAFF-R expression. (FIGS. 23 and 24).

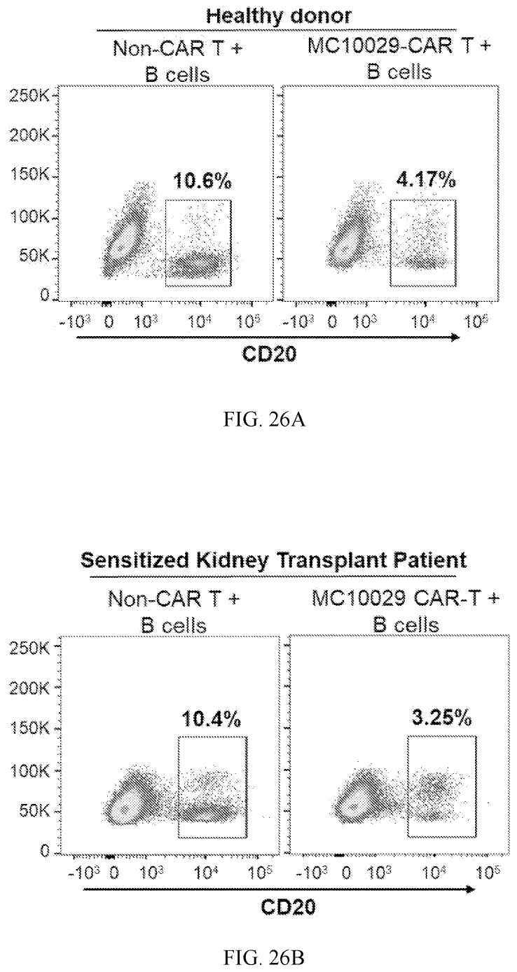

A direct killing assay using B cells as the target cells was initially established using healthy donor-derived MC10029 CAR T-cells. Upon the introduction of CAR T-cells, a notable reduction in the percentage of viable B cells was observed within the MC10029 CAR-T group compared to the non-CAR T group. The direct B cell killing by the MC10029 CAR T-cells derived from a representative sensitized kidney transplant patient (Patient 5) against B cells from healthy donor as the substitute target cells was then evaluated. A remarkable reduction in live CD20+B cells was observed (FIG. 26).

Of the ten enrolled patients, three patient samples (Patients 7-9) did not yield sufficient B cells for autologous experiments. However, patient-specific MC10029 CAR T-cells were successfully generated for these individuals (Table 3) and demonstrated the antigen-specific functionality of these CAR T-cells by using the set of BAFF-R-positive and BAFF-R-negative Nalm-6 cells as target cells (representative data for Patient 7 in FIG. 25) that was comparable to functionality observed for Patients 1-6.

Together these results demonstrate that MC10029 CAR T-cells can be used to treat organ transplant rejection. For example, MC10029 CAR T-cells generated from T cells derived from sensitized patients exhibited cytotoxic effects against autologous B cells and thus can be used to desensitization a mammal received an organ transplant (e.g., a kidney transplant) to the transplanted organ, thereby reducing or eliminating the risk of organ transplant rejection in that mammal.

### Example 19: Treating Cancer

Cells (e.g., T cells) designed to express one or more binders (e.g., antibodies, antigen binding fragments, antibody domains, CARs, or cell engagers) having the ability to bind to a BAFF-R polypeptide are administered to a human identified as having a B-cell cancer. The cells (e.g., T cells) engineered to express one or more binders (e.g., antibodies, antigen binding fragments, antibody domains, CARs, or cell engagers) having the ability to bind to a BAFF-R polypeptide are administered using intravenous injection. After the administration of cells (e.g., T cells) engineered to express one or more binders (e.g., antibodies, antigen binding fragments, antibody domains, CARs, or cell engagers) having the ability to bind to a BAFF-R polypeptide, the number of cancer cells (e.g., cancer cells expressing a BAFF-R polypeptide) within the human is reduced. After the administration of the cells (e.g., T cells) engineered to express one or more binders (e.g., antibodies, antigen binding fragments, antibody domains, CARs, or cell engagers) having the ability to bind to a BAFF-R polypeptide, the size of one or more tumors (e.g., tumors expressing a BAFF-R polypeptide) within the human is reduced.

### Example 20: Generation of T Cells Expressing One or More Binders Having the Ability to Bind to a BAFF-R Polypeptide

T cells are obtained from a human identified as having a B-cell cancer. Nucleic acid designed to express one or more binders (e.g., one or more CARs) having the ability to bind to a BAFF-R polypeptide provided herein is introduced into the T cell by transduction (e.g., viral transduction using a retroviral vector such as a lentiviral vector) or transfection such that the T cell expresses the binder(s) having the ability to bind to a BAFF-R polypeptide. The T cells engineered to express one or more binders (e.g., one or more CARs) having the ability to bind to a BAFF-R polypeptide are administered back into the human using, for example, intravenous injection. After the administration of the cells (e.g., T cells) engineered to express a one or more binders (e.g., one or more CARs) having the ability to bind to a BAFF-R polypeptide, the number of cancer cells (e.g., cancer cells expressing a BAFF-R polypeptide) within the human is reduced. After the administration of the cells (e.g., T cells) engineered to express a one or more binders (e.g., one or more CARs) having the ability to bind to a BAFF-R polypeptide, the size of one or more tumors (e.g., tumors expressing a BAFF-R polypeptide) within the human is reduced.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen binding fragment comprising:
a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

2. The antibody or antigen binding fragment of claim 1, wherein said antibody or antigen binding fragment comprises the ability to bind to SEQ ID NO:42.

3-11. (canceled)

12. A chimeric antigen receptor comprising an antigen binding domain, a hinge, a transmembrane domain, and one or more signaling domains, wherein said antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

13. The chimeric antigen receptor of claim 12, wherein said antigen binding domain comprises a scFv having the ability to bind to a B-cell activating factor receptor (BAFF-R) polypeptide.

14. The chimeric antigen receptor of claim 12, wherein said hinge comprises an amino acid sequence set forth in SEQ ID NO:30.

15. The chimeric antigen receptor of claim 12, wherein said transmembrane domain comprises an amino acid sequence set forth in SEQ ID NO:32.

16. The chimeric antigen receptor of claim 12, wherein said chimeric antigen receptor comprises one or more signaling domains set forth in any one of SEQ ID NOs:34 and 36.

17. A cell comprising:
(a) a chimeric antigen receptor comprising an antigen binding domain, a hinge, a transmembrane domain, and one or more signaling domains, wherein said antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11;
(b) nucleic acid comprising a nucleic acid sequence encoding at least part of an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11;
(c) nucleic acid comprising a nucleic acid sequence encoding said chimeric antigen receptor; or
(d) nucleic acid comprising a nucleic acid sequence encoding a cell engager comprising a first antigen binding domain, a linker, and a second antigen binding domain, wherein said first antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

18. The cell of claim 17, wherein said cell is a T cell, a stem cell, or an NK cell.

19. A cell engager comprising a first antigen binding domain, a linker, and a second antigen binding domain, wherein said first antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

20. The cell engager of claim 19, wherein said first antigen binding domain comprises a scFv or an IgG having the ability to bind to a BAFF-R polypeptide.

21-29. (canceled)

30. A nucleic acid comprising a nucleic acid sequence encoding:
(a) at least part of an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11;
(b) a chimeric antigen receptor comprising an antigen binding domain, a hinge, a transmembrane domain, and one or more signaling domains, wherein said antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11; or
(c) a cell engager comprising a first antigen binding domain, a linker, and a second antigen binding domain, wherein said first antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

31-40. (canceled)

41. An antibody-drug conjugate (ADC) comprising an antigen binding domain covalently linked to a drug, wherein said antigen binding domain comprises an antibody or an antigen binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

42-52. (canceled)

53. A method of treating a mammal having cancer or a transplanted organ, wherein said method comprises administering, to said mammal, a composition comprising:
(a) an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO: 11;
(b) a cell comprising a chimeric antigen receptor comprising an antigen binding domain, a hinge, a transmembrane domain, and one or more signaling domains, wherein said antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11;
(c) a cell engager comprising a first antigen binding domain, a linker, and a second antigen binding domain, wherein said first antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11; or
(d) an antibody-drug conjugate (ADC) comprising an antigen binding domain covalently linked to a drug, wherein said antigen binding domain comprises an antibody or an antigen binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

54. The method of claim 53, wherein said mammal is a human.

55. The method of claim 53, wherein said method comprising treating said mammal having cancer, and wherein said cancer is a BAFF-R⁺ cancer.

56. The method of claim 55, wherein said BAFF-R⁺ cancer is selected from the group consisting of a chronic lymphocytic leukemia (CLL), an acute lymphoblastic leukemia (ALL), a hairy cell leukemia, a follicular lymphoma, a non-Hodgkin's lymphoma (NHL), a Hodgkin's lymphoma, a multiple myeloma, a Waldenstrom's macroglobulinemia, a diffuse large B-cell lymphoma (DLBCL), and an intravascular large B-cell lymphoma.

57-61. (canceled)

62. The method of claim 55, wherein said composition further comprises a checkpoint inhibitor selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

63-75. (canceled)

76. The method of claim 53, wherein said method comprises treating said mammal having said transplanted organ, and wherein said transplanted organ is selected from the group consisting of a kidney, a lung, a liver, a heart, a pancreas, and bone marrow.

77. A method of delaying or preventing organ transplant rejection in a mammal having or preparing to have an organ transplant, wherein said method comprises administering, to said mammal, a composition comprising:
(a) an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11;
(b) a cell comprising a chimeric antigen receptor comprising an antigen binding domain, a hinge, a transmembrane domain, and one or more signaling domains, wherein said antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11;
(c) a cell engager comprising a first antigen binding domain, a linker, and a second antigen binding domain, wherein said first antigen binding domain comprises an antibody or an antigen-binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11; or
(d) an antibody-drug conjugate (ADC) comprising an antigen binding domain covalently linked to a drug, wherein said antigen binding domain comprises an antibody or an antigen binding fragment comprising a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO:11.

78-79. (canceled)
